(19) United States
(12) Patent Application Publication
(10) Pub. No.: US 2008/0038824 A1
(43) Pub. Date: Feb. 14, 2008

(54) Composition and Method for Elevating Gene Transfer Efficiency

(75) Inventors: Masato Miyake, Amagasaki-shi (JP); Tomohiro Yoshikawa, Amagasaki-shi (JP); Eiichiro Uchimura, Amagasaki-shi (JP); Jun Miyake, Amagasaki-shi (JP)

Correspondence Address: SEED INTELLECTUAL PROPERTY LAW GROUP PLLC, 701 FIFTH AVE, SUITE 5400, SEATTLE, WA, 98104, US

(73) Assignees: National Institute of Advanced Industrial Science and Technology, Chiyoda-ku (JP); Cytopathfinder, Inc., Shinagawa-ku (JP)

(21) Appl. No.: 10/587,776

(22) PCT Filed: Jan. 27, 2005
(86) PCT No.: PCT/JP05/01148

§ 371 Date: Jun. 18, 2007

(30) Foreign Application Priority Data
Jan. 29, 2004 (JP) 2004-022315

(51) Int. Cl. C12N 15/00; C07K 14/00; C07K 16/00; C07K 16/28

(52) U.S. Cl. 435/440; 435/285.1; 530/350; 530/387.1; 530/388.22; 530/389.1

(57) ABSTRACT

It is intended to provide a method whereby transfer efficiency can be improved under any conditions in the case of transferring (in particular, transfecting) a target substance that cannot easily be transferred into cells (for example, a DNA, a polypeptide, a sugar, a complex thereof or the like). Namely, a composition for elevating the transfer efficiency of a target substance into cells which contains a cell adhesion-related factor, and a device and a method using the composition.

### TECHNICAL FIELD

The present invention relates to the field of cell biology. More particularly, the present invention relates to a compound, composition, device, method and system for increasing the efficiency of introducing a substance into a cell.

### BACKGROUND ART

Techniques for introducing a protein into cells (i.e., transfection, transformation, transduction, etc.) are generally used in a wide variety of fields, such as cell biology, genetic engineering, molecular biology, and the like.

Transfection is conducted to temporarily express a gene in cells, such as animal cells and the like, so as to observe an influence of the gene. Since the advent of the post-genomic era, transfection techniques are frequently used to elucidate the functions of genes encoded by the genome.

Various techniques and agents used therein have been developed to achieve transfection. One technique employs a cationic substance, such as a cationic polymer, a cationic lipid, or the like, and is widely used.

In many cases, however, use of conventional agents is not sufficient for transfection efficiency. There has been conventionally no agent, which can be used either in solid phase or in liquid phase. Therefore, there is great demand for such an agent. Further, there is an increasing demand for a technique for efficiently introducing (e.g., transfecting, etc.) a target substance into cells or the like on a solid phase, such as microtiter plates, arrays, and the like.

The difficulty in transfecting cells or producing transgenic organisms hinders the progression of development of dominant negative screening in mammals. To overcome this problem, high-efficiency retrovirus transfection has been developed. Although this retrovirus transfection is potent, it is necessary to produce DNA to be packaged into viral intermediates, and therefore, the applicability of this technique is limited. Alternatively, high-density transfection arrays are being developed, but are not necessarily applicable to all cells. Various systems for liquid phase transfection have been developed. However, the efficiency of such techniques is low for adherent cells, for example. Thus, such techniques are not necessarily applicable to all cells.

As such, that the development of a transfection system which is applicable to any system or cell is greatly desired in the art. Demand for such a transfection system for use in mass high-through put assay using e.g., microtiterplates, arrays and the like, increases year on year, since such a system could be applied to a variety of cells and experimental systems.

### DISCLOSURE OF INVENTION

### Problems to be Solved by the Invention

Considering the above, it is an object of the present invention to develop a method for improving the introduction (in particular, transfection) efficiency of a target substance (e.g. DNA, polypeptide, sugar or a complex thereof or the like), which is difficult to introduce into a cell by means of conventional diffusion or hydrophobic interaction, under all circumstances in which it is desirable to introduce such.

### Means for Solving the Problems

Such an object has been fulfilled by the unexpected discovery that a system using a cellular adhesion related agent, such as an antibody against an integrin, significantly enhances the introduction efficiency of a target substance into a cell. Such an object has also been in part achieved by the discovery, that the inhibition of adhesion of a cell unexpectedly enhances the introduction of a target substance such as a gene, and also in the case of genetic materials and the like, that expression of such in the cell is observed.

Accordingly, the present invention provides the following:

1. A composition for enhancing the introduction efficiency of a target substance into a cell, comprising a cellular adhesion related agent.

2. A composition for enhancing the introduction efficiency of a target substance into a cell according to item 1, wherein the cellular adhesion related agent comprises an interaction substance interacting with a cellular adhesion molecule.

3. A composition according to item 2, wherein the cellular adhesion molecule is an extracellular matrix.

4. A composition according to item 2, wherein the cellular adhesion molecule is an integrin receptor.

5. A composition according to item 2, wherein the cellular adhesion molecule comprises an RGD molecule.

6. A composition according to item 2, wherein the interaction molecule raises an antigen-antibody reaction with a partner of the cellular adhesion molecule.

7. A composition according to item 2, wherein the interaction molecule is an antibody or a derivative thereof.

8. A composition according to item 2, wherein the interaction molecule is a monoclonal or polyclonal antibody.

9. A composition according to item 2, wherein the interaction molecule comprises an antibody selected from the group consisting of an anti-CD49a antibody, an anti-CD49b antibody, an anti-CD49c antibody, an anti-CD49e antibody, and an anti-CD49f antibody.

10. A composition according to item 1, wherein the target substance comprises a genetic material.

11. A composition according to item 1, wherein the target substance comprises a nucleic acid molecule.

12. A composition according to item 1, wherein the target substance comprises DNA.

13. A composition according to item 4, wherein the integrin receptor is selected from the group consisting of CD49a, CD49b, CD49c, CD49d, CD49e, CD49f and CD29.

14. A composition according to item 4, wherein the integrin receptor is selected from the group consisting of CD29, CD49a, CD49c, Cd49d, CD49e and CD49f.

15. A composition according to item 4, wherein the integrin receptor interacts with a molecule selected from the group consisting of collagen, fibronectin, vitronectin and laminin.

16. A composition according to item 1, wherein the cell comprises at least one cell selected from the group consisting of a stem cell and a differentiated cell.

17. A composition according to item 1, wherein the cellular adhesion molecule is specifically expressed in the cell.

18. A composition according to item 1, wherein the target substance is a genetic material and the composition further comprises a gene introduction reagent.

19. A composition according to item 18, wherein the gene introduction reagent is selected from the group consisting of a cationic macromolecule, cationic lipid and calcium phosphate.

20. A composition according to item 1, further comprising a particle.

21. A composition according to item 20, wherein the particle comprises a gold colloid.

22. A composition according to item 1 further comprising a salt.

23. A composition according to item 22, wherein the salt is selected from the group consisting of salts comprised in a buffer and salts comprised in media.

24. A kit for enhancing gene introduction efficiency, comprising:

(a) a cellular adhesion related agent; and

(b) a gene introduction reagent.

25. A composition for introducing a target material to a cell, comprising:

(A) a target material; and

(B) a cellular adhesion related agent.

26. A composition according to item 25, wherein the target material comprises a substance selected from the group consisting of DNA, RNA, polypeptide, sugar and a complex thereof.

27. A composition according to item 25, wherein the target material comprises a DNA encoding a gene sequence to be transfected into the cell.

28. A composition according to item 25 further comprising a gene introduction reagent.

29. A composition according to item 25, wherein the cellular adhesion related agent comprises an interaction substance interacting with a cellular adhesion molecule.

30. A composition according to item 25, wherein the cellular adhesion related agent comprises an antibody to a cellular adhesion molecule.

31. A composition according to item 25 which is present as a liquid phase.

32. A composition according to item 25 which is present as a solid phase.

33. A device for enhancing gene introduction efficiency of a target molecule into a cell, comprising:

(a) a target molecule; and

(b) a cellular adhesion related agent,

wherein the cellular adhesion related agent is immobilized onto a support.

34. A device according to item 33, wherein the target substance comprises a substance selected from the group consisting of DNA, RNA, polypeptide, sugar and a complex thereof.

35. A device according to item 33, wherein the target substance comprises a DNA encoding a gene sequence for the purpose of gene expression.

36. A device according to item 33, further comprising a gene introduction reagent.

37. A device according to claim 36, wherein the cellular adhesion related agent comprises an interaction substance interacting with a cellular adhesion molecule.

38. A device according to item 36, wherein the cellular adhesion related agent comprises an antibody against a cellular adhesion molecule.

39. A device according to item 36, wherein the support is selected from the group consisting of a plate, a microwell plate, a tip, a slide glass, a film, a bead and metal.

40. A device according to item 36, wherein the support is coated with a coating agent.

41. A device according to item 40, wherein the coating agent comprises a substance selected from the group consisting of poly-L-lysine, silane, MAS, hydrophobic fluorine resin and metal.

42. A method for enhancing the introduction efficiency of a target substance into a cell, comprising the steps of:

A) providing a target substance;

B) providing a cellular adhesion related agent; and

C) contacting the target substance and the cellular adhesion related substance with the cell.

43. A method according to item 42, wherein the target material comprises a substance selected from the group consisting of DNA, RNA, polypeptide, sugar and a complex thereof.

44. A method according to item 43, wherein the target material comprises a DNA encoding a gene sequence to be transfected in the cell.

45. A method according to item 42, further comprising a gene introduction reagent.

46. A method according to item 42, wherein the cellular adhesion related agent comprises an interaction substance interacting with a cellular adhesion molecule.

47. A method according to item 42, wherein the cellular adhesion related agent comprises an antibody to a cellular adhesion molecule.

48. A method according to item 46, wherein the cellular adhesion molecule is an extracellular matrix molecule.

49. A method according to item 42, wherein the method is conducted in a liquid phase.

50. A method according to item 42, wherein the method is conducted in a solid phase.

51. A method for enhancing the introduction efficiency of a target substance into a cell, comprising the steps of:

I) immobilizing a composition comprising
A) a target substance, and
B) a cellular adhesion molecule onto a support; and

II) contacting a cell to the composition on the support.

52. A method according to item 51, further comprising the step of providing a gene introduction reagent, said gene introduction reagent being contacted with the cell.

53. A method according to item 52, further comprising the step of forming a complex between the target substance and a gene introduction reagent after the provision thereof, wherein thereafter the cellular adhesion related agent is provided.

54. A method according to item 51, wherein the cellular adhesion related agent comprises an interaction substance interacting with a cellular adhesion molecule.

Hereinafter, the present invention will be described by way of preferred embodiments. It will be understood by those skilled in the art that the embodiments of the present invention can be appropriately made or carried out based on the description of the present specification and the accompanying drawings, and commonly used techniques well known in the art. The function and effect of the present invention can be easily recognized by those skilled in the art.

[symbol]

### EFFECTS OF INVENTION

The present invention enhances transfection efficiency and may be practiced in a solid or liquid phase. Such a transfection efficiency enhancing reagent is particularly useful when conducting transfection in a solid phase.

### BRIEF DESCRIPTION OF THE DRAWINGS

FIG. 1 depicts the relationship between an integrin receptor and an extracellular matrix molecule to be recognized thereby.

FIG. 2A depicts the results of a transfection efficiency test using a variety of cellular adhesion related agents and fibronectin, in an experiment using HepG2 cells.

FIG. 2B depicts another example results from transfection efficiency experiments using different cellular adhesion related agents in experiments using HepG2 cells.

FIG. 3A shows the relationship between a variety of integrin receptors (CD49a, CD49d and CD49f) expressed on HepG2 cells with extracellular matrix molecules, and transfection efficiency on the surfaces coated with specific extracellular matrix molecules.

FIG. 3B is a photograph of a cell exhibiting the state of transfection in an experiment using HepG2 cells. No coating refers to a control without coating treatment, and fibronectin and CD29 are also shown.

FIG. 3C is a photograph of a cell exhibiting the state of transfection in an experiment using HepG2 cells. CD44, CD46 and CD54 are shown.

FIG. 3D is photograph of a cell exhibiting the state of transfection in an experiment using HepG2 cells. CD49a, CD49c and CD49d are shown.

FIG. 3E is a photograph of a cell exhibiting the state of transfection in an experiment using HepG2 cells. CD49e, CD49f and HLA are shown.

FIG. 4 depicts the relationship between extracellular matrix molecules which are recognized to be a variety of integrin receptors (CD49a, d and f) expressed on a PC12 cell in Example 4; and the transfection efficiency on the surface coated with a variety of extracellular matrix molecules.

FIG. 5A-B depicts the cell adhesion of PC12 cells in Example 4. It shows adhesion inhibition and transfection of PC12 cells on a Type IV collagen coated surface using cell adhesion related agents (for example, CD antibodies). Type IV collagen was coated onto poly-L-lysine coated slides, and thereafter, PC12 cells which had been previously contacted with an antibody solution was seeded thereon and subsequently transfected with Lipofectamine 2000 according to the conventional protocol for use in liquid phase. FIG. 5A shows that anti-CD49a antibody significantly inhibited the adhesion of PC12 cells onto a Type IV collagen coated surface. An anti-CD49d antibody shows the similar transfection efficiency as the control without antibody, by exhibiting no inhibition of adhesion of PC12 cells. As seen in FIG. 4, PC12 cells do not express CD49d. Furthermore, CD49d is a receptor for fibronectin, and thus it is believed that the adhesion of the Type IV collagen coated surface is not inhibited. That is, the existence of an antibody per se does not affect the transfection efficiency. FIG. 5B similarly shows the difference in transfection efficiency in the presence of an anti-CD49f antibody. The values shown indicate the dilution of the antibody stock solution used to contact the PC12 cells.

FIG. 6 depicts the transfection efficiency of PC12 cells in the context of the coated support shown in Example 4. An anti-CD49a, anti-CD49c, and anti-CD49d antibodies were used.

FIG. 7 depicts the transfection efficiency of PC12 cells in the context of the coated support shown in Example 4. An anti-CD49e, anti-CD49f, and anti-HLA antibodies were used.

FIG. 8 depicts the outlines of solid transfection using a cellular adhesion related agent. The lower right-hand panel is the same ????? as in FIG. 1. The upper-right handed panel shows the fluorescence intensity distribution using three anti-CD antibodies. The left-hand panel shows the relative transfection efficiency indices.

### DESCRIPTION OF SEQUENCE LISTING

SEQ ID NO.: 1: a nucleic acid sequence encoding fibronectin (human)

SEQ ID NO.: 2: an amino acid sequence of fibronectin (human)

SEQ ID NO.: 3: an amino acid sequence of CD29 (isoform 1A precursor)

SEQ ID NO.: 4: an amino acid sequence of CD49a

SEQ ID NO.: 5: an amino acid sequence of CD49b

SEQ ID NO.: 6: an amino acid sequence of CD49c (isoform A precursor)

SEQ ID NO.: 7: an amino acid sequence of CD49d (precursor)

SEQ ID NO.: 8: an amino acid sequence of CD49e (precursor)

SEQ ID NO.: 9: an amino acid sequence of CD49f

SEQ ID NO.: 10: an amino acid sequence of CD29 (isoform 1B precursor)

SEQ ID NO.: 11: an amino acid sequence of CD29 (isoform 1C-1 precursor)

SEQ ID NO.: 12: an amino acid sequence of CD29 (isoform 1C-2 precursor)

SEQ ID NO.: 13: an amino acid sequence of CD29 (isoform 1D precursor)

SEQ ID NO. 14: an amino acid sequence of CD49c (isoform B precursor)

### BEST MODE FOR CARRYING OUT THE INVENTION

Hereinafter, the present invention will be described. It should be understood throughout the present specification that expressions for a singular form include the concept of their plurality unless otherwise mentioned. It should be also understood that the terms as used herein have definitions typically used in the art unless otherwise mentioned. Accordingly, unless otherwise defined, all technical and scientific terms used herein shall have the same meaning as that generally understood by those skilled in the art to which the present invention pertains. If there is any inconsistency, the present specification precedes, including definitions.

(Definition of Terms)

Hereinafter, terms specifically used herein will be defined.

As used herein, the terms “cell adhesion molecule” and “adhesion molecule” are used interchangeably to refer to a molecule capable of mediating the joining of two or more cells (cell adhesion) or adhesion between a substrate and a cell. In general, cell adhesion molecules are divided into two groups: molecules involved in cell-cell adhesion (intercellular adhesion) (cell-cell adhesion molecules) and molecules involved in cell-extracellular matrix adhesion (cell-substrate adhesion) (cell-substrate adhesion molecules). In the method of the present invention, any molecule may be useful and may be effectively used. Therefore, cell adhesion molecules herein include a substrate protein and a cellular protein (e.g., integrin, etc.) in cell-substrate adhesion. A molecules other than a protein falls within the concept of a cell adhesion molecule as long as it can mediate cell adhesion.

For cell-cell adhesion, cadherin, a number of molecules belonging to the immunoglobulin superfamily (NCAML1, ICAM, fasciclin II, III, etc.), selectin, and the like are known, each of which is known to join cell membranes via a specific molecular reaction.

As such, a variety of molecules are involved in cellular adhesion, and have different functions amongst each other, and thus those skilled in the art can appropriately select a molecule to be considered in the present invention, depending on the purpose thereof. Techniques for cell adhesion are well known, as described above and as described in, for example, “Saibogaimatorikkusu—Rinsho heno Oyo—[Extracellular matrix—Clinical Applications—], Medical Review.

It can be determined whether or not a certain molecule is a cell adhesion molecule, by an assay, such as biochemical quantification (an SDS-PAGE method, a labeled-collagen method, etc.), immunological quantification (an enzyme antibody method, a fluorescent antibody method, an immunohistological study, etc.), a PCR method, a hybridization method, or the like, in which a positive reaction is detected. Examples of such a cell adhesion molecule include, but are not limited to, collagen, integrin, fibronectin, laminin, vitronectin, fibrinogen, an immunoglobulin superfamily member (e.g., CD2, CD4, CD8, ICM1, ICAM2, VCAM1), selectin, cadherin, CD29, C49 families and the like. Most of these cell adhesion molecules simultaneously transmit into a cell an auxiliary signal for cell activation due to intercellular interaction, as well as mediating cell adhesion.

An example of a cell adhesion molecule is cadherin which is present in many cells capable of being fixed to tissue. Cadherin can be used in a preferred embodiment of the present invention. Examples of a cell adhesion molecule in blood cells and immune system cells which are not fixed to tissue, include, but are not limited to, immunoglobulin superfamily molecules (CD 2, LFA-3, ICAM-1, CD2, CD4, CD8, ICM1, ICAM2, VCAM1, etc.); integrin family molecules (LFA-1, Mac-1, gpIIbIIIa, p150, p95, VLA1, VLA2, VLA3, VLA4, VLA5, VLA6, etc.); selectin family molecules (L-selectin, E-selectin, P-selectin, etc.); CD29, CD49 families (CD49a, CD49b, CD49c, CD49d, CD49e, CD49f and the like), and the like. Such molecules may be useful for treating blood/immunological systems or organs.

A cellular adhesion molecule is necessary for immobilizing a non-adhesive cell. In such a case, it is believed that primary adhesion is due to selectin molecules or the like, which are permanently expressed in a cell, and subsequently, secondary adhesion due to integrin molecules or the like, is subsequently activated thereafter, which gradually strengthens the adhesion between cells. Accordingly, cellular adhesion molecules contemplated in the present invention may be considered to encompass an agent mediating such primary adhesion, an agent mediating secondary adhesion, or both in combination and it appears to be preferable to have secondary adhesion. When a cellular adhesion molecule is a protein, the active site thereof has already been elucidated at the amino acid level, and RGD and YIGSR motifs and the like are already known, and are collectively called an RGD sequence.

As used herein the term “cellular adhesion related agent” refers to an agent suppressing the adhesion of a cell to another substance such as a support, other cells or the like. Such an agent includes, but is not limited to interaction substances which interact with a cellular adhesion molecule. The adhesion suppression activity of such interaction substances may be confirmed by the co-existence of an interaction substance when a cell is seeded onto a surface coated with an ECM substrate such as fibronectin. Furthermore, when substrates having such interaction activity are chemically or physically immobilized onto a surface, progress of adhesion property onto the surface of the cell is confirmed. Interaction substances include, but are not limited to, for example, substances allosterically interacting with a competitor, a partner in an antigen-antibody reaction (an antibody when the partner is an antigen, and an antigen when the partner is an antibody), a partner in a receptor-ligand relationship (a ligand when the partner is a receptor, and a receptor when the partner is a ligand), and the like. In the present invention, as long as cellular adhesion is enhanced, the object of the present invention (introduction of a target substance) may be achieved and thus it is to be understood that such agents are not particularly limited to a specific embodiment.

As used herein, the term “RGD molecule” refers to a protein molecule comprising an amino acid sequence RGD (Arg-Gly-Asp) or a sequence having the same function as that of the sequence RGD. RGD molecules are characterized by comprising an amino acid sequence RGD, which is useful as an amino acid sequence of a cell adhesion active site of a cellular adhesive protein or another amino acid sequence having an equivalent function. The RGD sequence was found to be a cell adhesion site of fibronectin, and subsequently, a number of molecules having cellular adhesive activity were found, including collagen type I, laminin, vitronectin, fibrinogen, the von Willebrand factor, entactin, and the like. If a chemically synthesized RGD peptide is attached to a solid phase, the peptide exhibits cell adhesion activity. A biological molecule of the present invention may be a chemically synthesized RGD molecule. Examples of such an RGD molecule include, but are not limited to, a GRGDSP peptide in addition to the above-described naturally-occurring molecules. The RGD sequence is recognized by integrin (e.g., CD49 family, CD29 and the like) which is a cell adhesion molecule (and also a receptor). Therefore, a molecule having a functional equivalent to an RGD sequence can be identified by examining its interaction with integrin.

As used herein, the term “integrin” or “integrin receptor” are interchangeably used to refer to a transmembrane glycoprotein which is a receptor involved in cell adhesion. Integrins are located on cell surfaces and function when a cell adheres to an extracellular matrix. It is known that integrins are involved in cell-cell adhesion in the hemocyte system. Examples of such integrins include, but are not limited to, receptors for fibronectin, vitronectin, collagen, or the like; IIb/IIIa in platelets; Mac-1 in macrophages; LFA-1, VLA-1 to 6 in lymphocytes; PSA in fruit flies (Drosophila); and the like. Typically, integrins have a hetero dimer structure in which an α chain having a molecular weight of 130 kDa to 210 kDa and a β chain having a molecular weight of 95 kDa to 130 kDa are associated via a non-covalent bond. Examples of the α chain include, but are not limited to, α¹, α², α³, α⁴, α⁵, α⁶, α^{L}, α^{M}, α^{X}, α^{IIB}, α^{V}, α^{E}, and the like. Examples of the β chain include, but are not limited to, β₁, β₂, β₃, β₄, β₅, β₆, β₇, and the like.

Examples of such a hetero dimer include, but are not limited to, Gp IIb IIIa, VLA-1, VLA-2, VLA-3, VLA-4, VLA-5, VLA-6, CD51/CD29, LFA-1, Mac-1, p150, p90, a vitronectin receptor, β⁴ subfamily, β⁵ subfamily, β⁶ subfamily, LPAM-1, HML-1, and the like. Typically, it is often that the extracellular domain of the α chain has a divalent cation binding site, and the extracellular domain of the β chain has a cysteine-rich domain and the intracellular domain of the β chain has a tyrosine phosphorylation site. A recognition site of a binding ligand is often the RGD sequence. Therefore, integrin may be an RGD molecule.

As used herein, the term “extracellular matrix” (ECM) refers to a substance existing between somatic cells no matter whether the cells are epithelial cells or non-epithelial cells. Extracellular matrices are involved in supporting tissue as well as in the internal environmental structure essential for the survival of all somatic cells. Extracellular matrices are generally produced from connective tissue cells. Some extracellular matrices are secreted from cells possessing basal membrane, such as epithelial cells or endothelial cells. Extracellular matrices are roughly divided into fibrous components and matrices filling there between. Fibrous components include collagen fibers and elastic fibers. A basic component of matrices is a glycosaminoglycan (acidic mucopolysaccharide), most of which is bound to non-collagenous protein to form a proteoglycan polymer (acidic mucopolysaccharide-protein complex). In addition, matrices include glycoproteins, such as laminin of basal membrane, microfibrils around elastic fibers, fibers, fibronectins on cell surfaces, and the like. Particularly differentiated tissue has the same basic structure. For example, in hyaline cartilage, chondroblasts characteristically produce a large amount of cartilage matrices including proteoglycans. In bones, osteoblasts produce bone matrices which cause calcification. Examples of an extracellular matrix for use in the present invention include, but are not limited to, collagen, elastin, proteoglycan, glycosaminoglycan, fibronectin, laminin, elastic fiber, collagen fiber, and the like. When used in the present invention, the extracellular matrix preferably includes, but is not limited to: fibronectin, vitronectin, laminin, and the like.

In the present invention, any extracellular matrix proteins may be considered for use as long as the protein is an agent relating to cellular adhesion.

CD numbers are a nomenclature in which the cellular surface antigens of leukocytes were originally identified as a differentiation marker. CD49a is a1 subunit, which is an integrin a chain subunit (the amino acid sequence thereof is SEQ ID NO: 4). CD49b is a2 subunit, which is an integrin a chain subunit (the amino acid sequence thereof is SEQ ID NO: 5). CD49c is a3 subunit, which is an integrin a chain subunit (the amino acid sequence thereof is SEQ ID NO: 6 and 14). CD49d is a4 subunit, which is an integrin a chain subunit (the amino acid sequence thereof is SEQ ID NO: 7). CD49e is a5 subunit, which is an integrin a chain subunit (the amino acid sequence thereof is SEQ ID NO: 8). CD49f is a6 subunit, which is an integrin a chain subunit (the amino acid sequence thereof is SEQ ID NO: 9). CD29 is B1 subunit, which is an integrin 3 chain subunit (the amino acid sequence thereof is SEQ ID NO: 3, 10, 11, 12 and 13). CD44 is a hyaluronic acid binding protein. CD46 is a cellular adhesion agent called membrane complement protein (MCP). CD54 is ICAM-1, an intracellular adhesion molecule which binds cell to cell. HLA is a product of the major histocompatibility antigenic gene complex (HLA genes) complex.

(General Techniques)

Molecular biological techniques, biochemical techniques, and microorganism techniques as used herein are well known in the art and commonly used, and are described in, for example, Sambrook J. et al. (1989), Molecular Cloning: A Laboratory Manual, Cold Spring Harbor, 3rd Ed. (2001); Ausubel, F. M. (1987), Current Protocols in Molecular Biology, Greene Pub. Associates and Wiley-interscience; Ausubel, F. M. (1989), Short Protocols in Molecular Biology: A Compendium of Methods from Current Protocols in Molecular Biology, Greene Pub. Associates and Wiley-interscience; Innis, M. A. (1990), PCR Protocols: A Guide to Methods and Applications, Academic Press; Ausubel, F. M. (1992), Short Protocols in Molecular Biology: A Compendium of Methods from Current Protocols in Molecular Biology, Greene Pub. Associates; Ausubel, F. M. (1995), Short Protocols in Molecular Biology: A Compendium of Methods from Current Protocols in Molecular Biology, Greene Pub. Associates; Innis, M. A. et al. (1995), PCR Strategies, Academic Press; Ausubel, F. M. (1999), Short Protocols in Molecular Biology: A Compendium of Methods from Current Protocols in Molecular Biology, Wiley, and annual updates; Sninsky, J. J. et al. (1999), PCR Applications: Protocols for Functional Genomics, Academic Press; Special issue, Jikken Igaku [Experimental Medicine] “Idenshi Donyu & Hatsugenkaiseki Jikkenho [Experimental Method for Gene introduction & Expression Analysis]”, Yodo-sha, 1997; and the like. Relevant portions (or possibly the entirety) of each of these publications are herein incorporated by reference.

DNA synthesis techniques and nucleic acid chemistry for preparing artificially synthesized genes are described in, for example, Gait, M. J. (1985), Oligonucleotide Synthesis: A Practical Approach, IRL Press; Gait, M. J. (1990), Oligonucleotide Synthesis: A Practical Approach, IRL Press; Eckstein, F. (1991), Oligonucleotides and Analogues: A Practical Approach, IRL Press; Adams, R. L. et al. (1992), The Biochemistry of the Nucleic Acids, Chapman & Hall; Shabarova, Z. et al. (1994), Advanced Organic Chemistry of Nucleic Acids, Weinheim; Blackburn, G. M. et al. (1996), Nucleic Acids in Chemistry and Biology, Oxford University Press; Hermanson, G. T. (1996), Bioconjugate Techniques, Academic Press; and the like, related portions of which are herein incorporated by reference.

As used herein, the term “biological molecule” refers to a molecule relating to an organism and an aggregation thereof. As used herein, the term “biological” or “organism” refers to a biological organism, including, but being not limited to, an animal, a plant, a fungus, a virus, and the like. A biological molecule includes a molecule extracted from an organism and an aggregation thereof, though the present invention is not limited to this. Any molecule capable of affecting an organism and an aggregation thereof falls within the definition of a biological molecule. Therefore, low molecular weight molecules (e.g., low molecular weight molecule ligands, etc.) capable of being used as medicaments fall within the definition of a biological molecule as long as an effect on an organism is intended. Examples of such a biological molecule include, but are not limited to, a protein, a polypeptide, an oligopeptide, a peptide, a polynucleotide, an oligonucleotide, a nucleotide, a nucleic acid (e.g. DNA such as cDNA and genomic DNA; RNA such as mRNA), a polysaccharide, an oligosaccharide, a lipid, a low molecular weight molecule (e.g., a hormone, a ligand, an information transmitting substance, a low molecular weight organic molecule, etc.), and a composite molecule thereof (glycolipids, glycoproteins, lipoproteins, etc.), and the like. A biological molecule may include a cell itself or a portion of tissue as long as it is intended to be introduced into a cell. Preferably, a biological molecule may include a nucleic acid (DNA or RNA) or a protein. In another preferred embodiment, a biological molecule is a nucleic acid (e.g., genomic DNA or cDNA, or DNA synthesized by PCR or the like). In another preferred embodiment, a biological molecule may be a protein.

As used herein the term “genetic material” refers to any material responsible for genetic information. Such materials include, but are not limited to, for example, nucleic acids such as DNA, RNA, PNA, and proteins and the like. DNA may be in any form such as cDNA, genomic DNA, artificial DNA, RNAi and the like. RNA may be mRNA, tRNA, rRNA, RNAi and the like.

The terms “protein”, “polypeptide”, “oligopeptide” and “peptide” as used herein have the same meaning and refer to an amino acid polymer having any length. This polymer may be a straight, branched or cyclic chain.

An amino acid may be a naturally-occurring or nonnaturally-occurring amino acid, or a variant amino acid. The term may include amino acids assembled into a composite of a plurality of polypeptide chains. The term also includes a naturally-occurring or artificially modified amino acid polymer. Such modification includes, for example, disulfide bond formation, glycosylation, lipidation, acetylation, phosphorylation, or any other manipulation or modification (e.g., conjugation with a labeling moiety). This definition encompasses a polypeptide containing at least one amino acid analog (e.g., nonnaturally-occurring amino acid, etc.), a peptide-like compound (e.g., peptoid), and other variants known in the art, for example. A gene product, such as an extracellular matrix protein (e.g., fibronectin, etc.), is in the form of a typical polypeptide.

The terms “polynucleotide”, “oligonucleotide”, and “nucleic acid” as used herein have the same meaning and refer to a nucleotide polymer having any length. This term also includes an “oligonucleotide derivative” or a “polynucleotide derivative”. An “oligonucleotide derivative” or a “polynucleotide derivative” includes a nucleotide derivative, or refers to an oligonucleotide or a polynucleotide having different linkages between nucleotides from typical linkages, which are interchangeably used. Examples of such an oligonucleotide specifically include 2′-O-methyl-ribonucleotide, an oligonucleotide derivative in which a phosphodiester bond in an oligonucleotide is converted to a phosphorothioate bond, an oligonucleotide derivative in which a phosphodiester bond in an oligonucleotide is converted to a N3′-P5′ phosphoroamidate bond, an oligonucleotide derivative in which a ribose and a phosphodiester bond in an oligonucleotide are converted to a peptide-nucleic acid bond, an oligonucleotide derivative in which uracil in an oligonucleotide is substituted with C-5 propynyl uracil, an oligonucleotide derivative in which uracil in an oligonucleotide is substituted with C-5 thiazole uracil, an oligonucleotide derivative in which cytosine in an oligonucleotide is substituted with C-5 propynyl cytosine, an oligonucleotide derivative in which cytosine in an oligonucleotide is substituted with phenoxazine-modified cytosine, an oligonucleotide derivative in which ribose in DNA is substituted with 2′-O-propyl ribose, and an oligonucleotide derivative in which ribose in an oligonucleotide is substituted with 2′-methoxyethoxy ribose. Unless otherwise indicated, a particular nucleic acid sequence also implicitly encompasses conservatively-modified variants thereof (e.g. degenerate codon substitutions) and complementary sequences as well as the sequence explicitly indicated. Specifically, degenerate codon substitutions may be produced by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). A gene for an extracellular matrix protein (e.g., fibronectin, etc.) is in the form of a typical polynucleotide. A polynucleotide may be used for transfection.

As used herein, the term “nucleic acid molecule” is used interchangeably with “nucleic acid”, “oligonucleotide”, and “polynucleotide” and includes cDNA, mRNA, genomic DNA, and the like. As used herein, a nucleic acid and a nucleic acid molecule may be included in the concept of the term “gene”. A nucleic acid molecule encoding the sequence of a given gene includes a “splice mutant (variant)”. Similarly, a particular protein encoded by a nucleic acid encompasses any protein encoded by a splice variant of that nucleic acid. “Splice mutants”, as the name suggests, are products of alternative splicing of a gene. After transcription, an initial nucleic acid transcript may be spliced such that different (alternative) nucleic acid splice products encode different polypeptides. Mechanisms for the production of splice variants vary, but include alternative splicing of exons. Alternative polypeptides derived from the same nucleic acid by read-through transcription are also encompassed by this definition. Any products of a splicing reaction, including recombinant forms of the splice products, are included in this definition.

As used herein, the term “gene” refers to an element defining a genetic trait. A gene is typically arranged in a given sequence on a chromosome. A gene which defines the primary structure of a protein is called a structural gene. A gene which regulates the expression of a structural gene is called a regulatory gene (e.g., promoter). Genes herein include structural genes and regulatory genes unless otherwise specified. Therefore, a fibronectin gene typically includes both a structural gene for fibronectin and a promoter for fibronectin. As used herein, “gene” may refer to “polynucleotide”, “oligonucleotide”, “nucleic acid”, and “nucleic acid molecule” and/or “protein”, “polypeptide”, “oligopeptide” and “peptide”. As used herein, “gene product” includes “polynucleotide”, “oligonucleotide”, “nucleic acid” and “nucleic acid molecule” and/or “protein”, “polypeptide”, “oligopeptide” and “peptide”, which are expressed by a gene. Those skilled in the art understand what a gene product is, according to the context.

As used herein, the term “homology” in relation to a sequence (e.g., a nucleic acid sequence, an amino acid sequence, etc.) refers to the proportion of identity between two or more gene sequences. Therefore, the greater the homology between two given genes, the greater the identity or similarity between their sequences. Whether or not two genes have homology is determined by comparing their sequences directly or by a hybridization method under stringent conditions. When two gene sequences are directly compared with each other, these genes have homology if the DNA sequences of the genes have representatively at least 50% identity, preferably at least 70% identity, more preferably at least 80%, 90%, 95%, 96%, 97%, 98%, or 99% identity with each other. As used herein, the term “similarity” in relation to a sequence (e.g., a nucleic acid sequence, an amino acid sequence, or the like) refers to the proportion of identity between two or more sequences when conservative substitution is regarded as positive (identical) in the above-described homology. Therefore, homology and similarity differ from each other in the presence of conservative substitutions. If no conservative substitutions are present, homology and similarity have the same value.

The similarity, identity and homology of amino acid sequences and base sequences are herein compared using FASTA (sequence analyzing tool) with the default parameters.

As used herein, the term “amino acid” may refer to a naturally-occurring or nonnaturally-occurring amino acid as long as the object of the present invention is satisfied. The term “amino acid derivative” or “amino acid analog” refers to an amino acid which is different from a naturally-occurring amino acid and has a function similar to that of the original amino acid. Such amino acid derivatives and amino acid analogs are well known in the art. The term “naturally-occurring amino acid” refers to an L-isomer of a naturally-occurring amino acid. The naturally-occurring amino acids are glycine, alanine, valine, leucine, isoleucine, serine, methionine, threonine, phenylalanine, tyrosine, tryptophan, cysteine, proline, histidine, aspartic acid, asparagine, glutamic acid, glutamine, γ-carboxyglutamic acid, arginine, ornithine, and lysine. Unless otherwise indicated, all amino acids as used herein are L-isomers. An embodiment using a D-isomer of an amino acid falls within the scope of the present invention. The term “nonnaturally-occurring amino acid” refers to an amino acid which is ordinarily not found in nature. Examples of nonnaturally-occurring amino acids include D-form of the amino acids described above, norleucine, para-nitrophenylalanine, homophenylalanine, para-fluorophenylalanine, 3-amino-2-benzyl propionic acid, D- or L-homoarginine, and D-phenylalanine. The term “amino acid analog” refers to a molecule having a physical property and/or function similar to that of amino acids, but which is not an amino acid. Examples of amino acid analogs include, for example, ethionine, canavanine, 2-methylglutamine, and the like. An amino acid mimic refers to a compound which has a structure different from that of the general chemical structure of amino acids but which functions in a manner similar to that of naturally-occurring amino acids.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As used herein, the term “corresponding” amino acid or nucleic acid refers to an amino acid or nucleotide in a given polypeptide or polynucleotide molecule, which has, or is anticipated to have, a function similar to that of a predetermined amino acid or nucleotide in a polypeptide or polynucleotide as a reference for comparison. Particularly, in the case of enzyme molecules, the term refers to an amino acid which is present at a similar position in an active site and similarly contributes to catalytic activity. For example, the binding domain in an integrin used in the present invention may be a portion (domain) in an ortholog corresponding to a molecule containing the domain.

As used herein, the term “nucleotide” may be either naturally-occurring or nonnaturally-occurring. The term “nucleotide derivative” or “nucleotide analog” refers to a nucleotide which is different from naturally-occurring nucleotides and has a function similar to that of the original nucleotide. Such nucleotide derivatives and nucleotide analogs are well known in the art. Examples of such nucleotide derivatives and nucleotide analogs include, but are not limited to, phosphorothioate, phosphoramidate, methylphosphonate, chiral-methylphosphonate, 2-O-methyl ribonucleotide, and peptide-nucleic acid (PNA).

As used herein, the term “fragment” with respect to a polypeptide or polynucleotide refers to a polypeptide or polynucleotide having a sequence length ranging from 1 to n−1 with respect to the full length of the reference polypeptide or polynucleotide (of length n). The length of the fragment can be appropriately changed depending on the purpose. For example, in the case of polypeptides, the lower limit of the length of the fragment includes 3, 4, 5, 6, 7, 8, 9, 10, 15, 20, 25, 30, 40, 50 or more nucleotides. Lengths represented by integers which are not herein specified (e.g., 11 and the like) may be appropriate as a lower limit. For example, in the case of polynucleotides, the lower limit of the length of the fragment includes 5, 6, 7, 8, 9, 10, 15, 20, 25, 30, 40, 50, 75, 100 or more nucleotides. Lengths represented by integers which are not herein specified (e.g., 11 and the like) may be appropriate as a lower limit. As used herein, the length of polypeptides or polynucleotides can be represented by the number of amino acids or nucleic acids, respectively. However, the above-described numbers are not absolute. The above-described numbers as the upper or lower limit are intended to include some greater or smaller numbers (e.g., ±10%), as long as the same function is maintained. For this purpose, “about” may be herein put ahead of the numbers. However, it should be understood that the interpretation of numbers is not affected by the presence or absence of “about” in the present specification. In the present invention, a fragment preferably has a certain size or greater (e.g., 5 kDa or more, etc.). Though not wishing to be bound by any theory, it is considered that a certain size is required for a fragment to act as a cellular adhesion related agent.

As used herein, “polynucleotides hybridizing under stringent conditions” refers to conditions commonly used and well known in the art. Such a polynucleotide can be obtained by conducting colony hybridization, plaque hybridization, Southern blot hybridization, or the like using a polynucleotide selected from the polynucleotides of the present invention. Specifically, a filter on which DNA derived from a colony or plaque is immobilized is used to conduct hybridization at 65° C. in the presence of 0.7 to 1.0 M NaCl. Thereafter, a 0.1 to 2-fold concentration SSC (saline-sodium citrate) solution (1-fold concentration SSC solution is composed of 150 mM sodium chloride and 15 mM sodium citrate) is used to wash the filter at 65° C. Polynucleotides identified by this method are referred to as “polynucleotides hybridizing under stringent conditions”. Hybridization can be conducted in accordance with a method described in, for example, Molecular Cloning 2nd ed., Current Protocols in Molecular Biology, Supplement 1-38, DNA Cloning 1: Core Techniques, A Practical Approach, Second Edition, Oxford University Press (1995), and the like. Here, sequences hybridizing under stringent conditions exclude, preferably, sequences containing only A or T. “Hybridizable polynucleotide” refers to a polynucleotide which can hybridize to other polynucleotides under the above-described hybridization conditions. Specifically, the hybridizable polynucleotide includes at least a polynucleotide having a homology of at least 60% to the base sequence of DNA encoding a polypeptide having an amino acid sequence specifically herein disclosed, preferably a polynucleotide having a homology of at least 80%, and more preferably a polynucleotide having a homology of at least 95%.

As used herein, the term “RNAi” is an abbreviation of RNA interference and refers to a phenomenon where an agent for causing RNAi, such as double-stranded RNA (also called dsRNA), is introduced into cells and mRNA homologous thereto is specifically degraded, so that the synthesis of gene products is suppressed, and techniques using the phenomenon. As used herein, RNAi may have the same meaning as that of an agent which causes RNAi.

As used herein, the term “an agent causing RNAi” refers to any agent capable of causing RNAi. As used herein, “an agent causing RNAi of a gene” indicates that the agent causes RNAi relating to the gene and that the effect of RNAi is achieved (e.g., suppression of expression of the gene, and the like). Examples of such an agent causing RNAi include, but are not limited to, a sequence having at least about 70% homology to the nucleic acid sequence of a target gene or a sequence hybridizable thereto under stringent conditions, RNA containing a double-stranded portion having a length of at least 10 nucleotides or variants thereof. Here, this agent may be preferably DNA containing a 3′ protruding end, and more preferably the 3′ protruding end has a length of 2 or more nucleotides (e.g. 2-4 nucleotides in length).

Though not wishing to be bound by any theory, the mechanism of RNAi is considered to be as follows. When a molecule which causes RNAi, such as dsRNA, is introduced into a cell, an RNaseIII-like nuclease having a helicase domain (called dicer) cleaves the molecule at about 20 base pair intervals from the 3′ terminus in the presence of ATP in the case where the RNA is relatively long (e.g., 40 or more base pairs). As used herein, the term “siRNA” is an abbreviation of short interfering RNA and refers to short double-stranded RNA of 10 or more base pairs which are artificially chemically synthesized or biochemically synthesized, synthesized by an organism, or produced by double-stranded RNA of about 40 or more base pairs being degraded within the organism. siRNA typically has a structure comprising 5′-phosphate and 3′-OH, where the 3′ terminus projects by about 2 bases. A specific protein is bound to siRNA to form RISC(RNA-induced-silencing-complex). This complex recognizes and binds to mRNA having the same sequence as that of siRNA and cleaves mRNA at the middle of siRNA due to RNaseIII-like enzymatic activity. It is preferable that the sequence of siRNA and the sequence of mRNA to be cleaved as a target is a 100% match. However, base mutations at a site away from the middle of siRNA do not completely remove the cleavage activity by RNAi, leaving partial activity, while base mutations in the middle of siRNA have a large influence and the mRNA cleavage activity by RNAi is considerably lowered. By utilizing such a phenomenon, only mRNA having a mutation can be specifically degraded. Specifically, siRNA in which the mutation is provided in the middle thereof is synthesized and is introduced into a cell. Therefore, in the present invention, siRNA per se, as well as an agent capable of producing siRNA (e.g., representatively dsRNA of about 40 or more base pairs) can be used as an agent capable of eliciting RNAi.

Also, though not wishing to be bound by any theory, apart from the above-described pathway, the antisense strand of siRNA binds to mRNA and siRNA functions as a primer for RNA-dependent RNA polymerase (RdRP), so that dsRNA is synthesized. This dsRNA is a substrate for a dicer again, leading to production of new siRNA. It is intended that such a reaction is amplified. Therefore, in the present invention, siRNA per se, as well as an agent capable of producing siRNA is useful. In fact, in insects and the like, for example, 35 dsRNA molecules can substantially completely degrade 1,000 or more copies of intracellular mRNA, and therefore, it will be understood that siRNA per se, as well as an agent capable of producing siRNA, is useful.

In the present invention, double-stranded RNA having a length of about 20 bases (e.g., representatively about 21 to 23 bases) or less than about 20 bases, called siRNA, can be used. Expression of siRNA in cells can suppress expression of a pathogenic gene targeted by the siRNA. Therefore, siRNA can be used for the treatment, prophylaxis, prognosis, and the like, of disease.

The siRNA of the present invention may be in any form as long as it can elicit RNAi.

In another embodiment, an agent capable of causing RNAi may have a short hairpin structure having a sticky portion at the 3′ terminus (shRNA; short hairpin RNA). As used herein, the term “shRNA” refers to a molecule of about 20 or more base pairs in which a single-stranded RNA partially contains a palindromic base sequence and forms a double-strand structure therein (i.e., a hairpin structure). shRNA can be artificially chemically synthesized. Alternatively, shRNA can be produced by linking sense and antisense strands of a DNA sequence in reverse directions and synthesizing RNA in vitro with T7 RNA polymerase using the DNA as a template. Though not wishing to be bound by any theory, it should be understood that after shRNA is introduced into a cell, the shRNA is degraded in the cell to a length of about 20 bases (e.g., representatively 21, 22, 23 bases), and causes RNAi as with siRNA, leading to the treatment effects of the present invention. It should be understood that such an effect is exhibited in a wide range of organisms, such as insects, plants, animals (including mammals), and the like. Thus, shRNA elicits RNAi as with siRNA and therefore can be used as an effective component of the present invention. shRNA may preferably have a 3′ protruding end. The length of the double-stranded portion is not particularly limited, but is preferably about 10 or more nucleotides, and more preferably about 20 or more nucleotides. Here, the 3′ protruding end may be preferably DNA, more preferably DNA of at least 2 nucleotides in length, and even more preferably DNA of 2-4 nucleotides in length.

An agent capable of causing RNAi used in the present invention may be artificially synthesized (chemically or biochemically) or naturally occurring. There is substantially no difference therebetween the two in terms of the effect of the present invention. A chemically synthesized agent is preferably purified by liquid chromatography or the like.

An agent capable of causing RNAi used in the present invention can be produced in vitro. In this synthesis system, T7 RNA polymerase and T7 promoter are used to synthesize antisense and sense RNAs from template DNA. These RNAs are annealed and thereafter introduced into a cell. In this case, RNAi is caused via the above-described mechanism, thereby achieving the effect of the present invention. Here, for example, the introduction of RNA into cell can be carried out using a calcium phosphate method.

Another example of an agent capable of causing RNAi according to the present invention is a single-stranded nucleic acid hybridizable to mRNA, or a nucleic acid analog thereof. Such agents are useful for the method and composition of the present invention.

As used herein, the term “salt” has the same meaning as that commonly understood by those skilled in the art, including both inorganic and organic salts. Salts are typically generated by neutralizing reactions between acids and bases. Salts include NaCl, K₂SO₄, and the like, which are generated by neutralization, and in addition, PbSO₄, ZnCl₂, and the like, which are generated by reactions between metals and acids. The latter salts may not be generated directly by neutralizing reactions, but may be regarded as a product of neutralizing reactions between acids and bases. Salts may be divided into the following categories: normal salts (salts that contain nether the H of an acid nor the OH of a base, including, for example, NaCl, NH₄Cl, CH₃COONa, and Na₂CO₃), acid salts (salts containing a remaining H of an acid, including, for example, NaHCO₃, KHSO₄, and CaHPO₄), and basic salts (salts containing a remaining OH of a base, including, for example, MgCl(OH) and CuCl(OH)). This classification is not very important in the present invention. Examples of preferable salts include salts contained in cell-culture media (e.g., calcium chloride, sodium hydrogen phosphate, sodium hydrogen carbonate, sodium pyruvate, HEPES, sodium chloride, potassium chloride, magnesium sulfide, iron nitrate, amino acids, vitamins, etc.), salts contained in buffer solutions (e.g., calcium chloride, magnesium chloride, sodium hydrogen phosphate, sodium chloride, etc.), and the like. These salts are preferable as they have a high affinity for cells and thus are better able to maintain cells in culture. These salts may be used singly or in combination. Preferably, these salts may be used in combination. This is because a combination of salts tends to have a higher affinity for cells. Therefore, a plurality of salts (e.g., calcium chloride, magnesium chloride, sodium hydrogen phosphate, and sodium chloride) are preferably contained in cell-culture medium, rather than only NaCl, or the like. More preferably, all salts for cell culture medium may be added to the medium. In another preferred embodiment, glucose may be added to medium.

As used herein, the term “search” indicates that a given nucleic acid sequence is utilized to find other nucleic acid base sequences having a specific function and/or property either electronically or biologically, or using other methods. Examples of an electronic search include, but are not limited to, BLAST (Altschul et al., J. Mol. Biol. 215:403-410 (1990)), FASTA (Pearson & Lipman, Proc. Natl. Acad. Sci., USA 85:2444-2448 (1988)), Smith and Waterman method (Smith and Waterman, J. Mol. Biol. 147:195-197 (1981)), and Needleman and Wunsch method (Needleman and Wunsch, J. Mol. Biol. 48:443-453 (1970)), and the like. Examples of a biological search include, but are not limited to, a macroarray in which genomic DNA is attached to a nylon membrane, or the like, or a microarray (microassay) in which genomic DNA is attached to a glass plate under stringent hybridization, PCR and in situ hybridization, and the like. It will be understood that Fn1 includes corresponding genes identified by such an electronic or biological search.

As used herein, the term “introduction” of a substance into a cell indicates that the substance enters the cell through the cell membrane. It can be determined whether or not the substance is successfully introduced into the cell, as follows. For example, the substance is labeled (e.g., with a fluorescent label, a chemoluminescent label, a phosphorescent label, a radioactive label, etc.) and the label is detected. Alternatively, changes in the cell, which are attributed to the substance (e.g., gene expression, signal transduction, events caused by binding to intracellular receptors, changes in metabolism, etc.), are measured physically (e.g., visual inspection, etc.), chemically (e.g., measurement of secreted substances, etc.), biochemically, or biologically. Therefore, the term “introduction” encompasses transfection, transformation, transduction and the like, which are usually called genetic manipulations, as well as transferring of substances, such as proteins, into cells.

As used herein, the term “target substance” refers to a substance which is intended to be introduced into cells. Substances targeted by the present invention are substances which are not introduced under normal conditions. Therefore, substances which can be introduced into cells by diffusion or hydrophobic interaction under normal conditions, are not targeted in an important aspect of the present invention. Examples of substances which are not introduced into cells under normal conditions, include, but are not limited to, proteins (polypeptides), RNA, DNA, sugars (particularly, polysaccharides), and composite molecules thereof (e.g., glycoproteins, PNA, etc.), viral vectors, and other compounds.

As used herein, the term “antibody” is used in the same manner as is conventionally used in the art, and encompasses polyclonal antibodies, monoclonal antibodies, human antibodies, humanized antibodies, polyfunctional antibodies, chimeric antibodies, and anti-idiotype antibodies, and fragments thereof (e.g., F(ab′)2 and Fab fragments), and other recombinant conjugates. These antibodies may be fused with an enzyme (e.g., alkaline phosphatase, horseradish peroxidase, α-galactosidase, and the like) via a covalent bond or by recombination. Preferably, an antibody recognizing the polypeptide used in the present invention is used, and more preferably, an antibody specifically recognizing the polypeptide used in the present invention is used. Such an antibody may be polyclonal or monoclonal. In an embodiment of the present invention, such an antibody is encompassed within the scope of the present invention.

As used herein the term “antigen” refers to a substance which causes immunological reactions such as antibody production and/or cytotoxicity and the like, by binding to an antibody or a specific receptor such as B-lymphocyte or T-lymphocyte or the like. The binding property with an antibody or a lymphocyte receptor is referred to as “antigenicity”. The property to induce immunological response such as antigen production is referred to as “immunogenicity”. Substances used as an antigen include, for example, at least one substance of interest such as a protein. The substances to be included therein is preferably of a full length sequence, however, it may also be of a partial sequence as long as the partial sequence includes at least one epitope capable of raising immunogenicity. As used herein the terms “epitope” or “antigen determinant” refer to a site in an antigen molecule, to which an antibody or a lymphocyte receptor binds. A method of determining an epitope is well known in the art, and such an epitope may be determined by means of well known or routine technology by those skilled in the art once a primary sequence of the nucleic acid or amino acid sequence is provided.

As used herein the term “partner of cell adhesion molecule” refers to a molecule which specifically interacts with a cellular adhesion molecule. As used herein, “interacting specifically with” a cellular adhesion agent/molecule refers to interaction with an affinity with the cellular adhesion agent/molecule higher than that of other substances than the particular cellular adhesion agent/molecule interacts with.

As used herein, the term “device” refers to a part which can constitute the whole or a portion of an apparatus, and comprises a support (preferably, a solid phase support) and a target substance carried thereon. Examples of such a device include, but are not limited to, chips, arrays, microtiter plates, cell culture plates, Petri dishes, films, beads, and the like.

As used herein, the term “support” refers to a material which can fix a substance, such as a biological molecule. Such a support may be made from any fixing material which has the capability of binding to a biological molecule as used herein via covalent or noncovalent bonding, or which may be induced to have such a capability.

Examples of materials used for supports include any material capable of forming a solid surface, such as, without limitation, glass, silica, silicon, ceramics, silicon dioxide, plastics, metals (including alloys), naturally-occurring and synthetic polymers (e.g., polystyrene, cellulose, chitosan, dextran, and nylon), and the like. A support may be formed of layers made of a plurality of materials. For example, a support may be made of an inorganic insulating material, such as glass, quartz glass, alumina, sapphire, forsterite, silicon oxide, silicon carbide, silicon nitride, or the like. A support may be made of an organic material, such as polyethylene, ethylene, polypropylene, polyisobutylene, polyethylene terephthalate, unsaturated polyester, fluorine-containing resin, polyvinyl chloride, polyvinylidene chloride, polyvinyl acetate, polyvinyl alcohol, polyvinyl acetal, acrylic resin, polyacrylonitrile, polystyrene, acetal resin, polycarbonate, polyamide, phenol resin, urea resin, epoxy resin, melamine resin, styrene-acrylonitrile copolymer, acrylonitrile-butadiene-styrene copolymer, silicone resin, polyphenylene oxide, polysulfone, and the like. Also in the present invention, nitrocellulose film, nylon film, PVDF film, or the like, which are used in blotting, may be used as a material for a support. When a material constituting a support is in a solid phase, such as a support is herein particularly referred to as a “solid phase support”. A solid phase support may be herein in the form of a plate, a microwell plate, a chip, a glass slide, a film, beads, a metal (surface), or the like. A support may or may not be coated.

As used herein, the term “liquid phase” has the same meaning as commonly understood by those skilled in the art, typically referring a state in solution.

As used herein, the term “solid phase” has the same meaning as commonly understood by those skilled in the art, typically referring to a solid state. As used herein, liquid and solid may be collectively referred to as a “fluid”.

As used herein, the term “contact” means that two substances (e.g., a compositions and a cell) are sufficiently close to each other that the two substances interact with each other.

As used herein, the term “interaction” refers to, without limitation, hydrophobic interactions, hydrophilic interactions, hydrogen bonds, Van der Waals forces, ionic interactions, nonionic interactions, electrostatic interactions, and the like. Preferably, interaction may be a typical interaction which takes place in organisms, such as a hydrogen bond, a hydrophobic interaction, or the like.

As used herein the term “interaction (or interacting) substance” refers to a substance which interacts with a certain target. Such a substance includes, but is not limited to, for example, an antibody against an antigen, a ligand against a receptor, or vice versa.

(Modification of Genes)

A cellular adhesion molecule or the like used in the present invention is often used in the form of a gene product. It will be understood that such a gene product may be a variant thereof. Therefore, substances produced using the gene modification techniques described below can be used in the present invention.

In a given protein molecule, a given amino acid may be substituted with another amino acid in a structurally important region, such as a cationic region or a substrate molecule binding site, without a clear reduction or loss of interactive binding ability. A given biological function of a protein is defined by the interactive ability or other property of the protein. Therefore, a particular amino acid substitution may be performed in an amino acid sequence, or at the DNA sequence level, to produce a protein which maintains the original property after the substitution. Therefore, various modifications of peptides as disclosed herein and DNA encoding such peptides may be performed without clear losses of biological activity.

When the above-described modifications are designed, the hydrophobicity indices of amino acids may be taken into consideration. The hydrophobic amino acid indices play an important role in providing a protein with an interactive biological function, which is generally recognized in the art (Kyte, J. and Doolittle, R. F., J. Mol. Biol. 157(1):105-132, 1982). The hydrophobic property of an amino acid contributes to the secondary structure of a protein and then regulates interactions between the protein and other molecules (e.g., enzymes, substrates, receptors, DNA, antibodies, antigens, etc.). Each amino acid is given a hydrophobicity index based on the hydrophobicity and charge properties thereof as follows: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamic acid (−3.5); glutamine (−3.5); aspartic acid (−3.5); asparagine (−3.5); lysine (−3.9); and arginine (−4.5).

It is well known that if a given amino acid is substituted with another amino acid having a similar hydrophobicity index, the resultant protein may still have a biological function similar to that of the original protein (e.g., a protein having an equivalent enzymatic activity). For such an amino acid substitution, the hydrophobicity index is preferably within ±2, more preferably within ±1, and even more preferably within ±0.5.

It is understood in the art that hydrophobicity is considered in the modification of a protein. As described in U.S. Pat. No. 4,554,101, amino acid residues are given the following hydrophilicity indices: arginine (+3.0); lysine (+3.0); aspartic acid (+3.0±1); glutamic acid (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). It is understood that an amino acid may be substituted with another amino acid which has a similar hydrophilicity index and can still provide a biological equivalent. For such an amino acid substitution, the hydrophilicity index is preferably within ±2, more preferably ±1, and even more preferably ±0.5.

The term “conservative substitution” as used herein refers to amino acid substitution in which a substituted amino acid and a substituting amino acid have similar hydrophilicity indices or/and hydrophobicity indices. For example, the conservative substitution is carried out between amino acids having a hydrophilicity or hydrophobicity index of within ±2, preferably within ±1, and more preferably within ±0.5. Examples of conservative substitution include, but are not limited to, substitutions within each of the following residue pairs: arginine and lysine; glutamic acid and aspartic acid; serine and threonine; glutamine and asparagine; and valine, leucine, and isoleucine, which are well known to those skilled in the art.

As used herein, the term “variant” refers to a substance, such as a polypeptide, polynucleotide, or the like, which differs partially from the original substance. Examples of such a variant include a substitution variant, an addition variant, a deletion variant, a truncated variant, an allelic variant, and the like. Examples of such a variant include, but are not limited to, a nucleotide or polypeptide having one or several substitutions, additions and/or deletions or a nucleotide or polypeptide having at least one substitution, addition and/or deletion. The term “allele” as used herein refers to a genetic variant located at a locus identical to a corresponding gene, where the two genes are distinguished from each other. Therefore, the term “allelic variant” as used herein refers to a variant which has an allelic relationship with a given gene. Such an allelic variant ordinarily has a sequence the same as or highly similar to that of the corresponding allele, and ordinarily has almost the same biological activity, though it rarely has different biological activity. The term “species homolog” or “homolog” as used herein refers to one that has an amino acid or nucleotide homology with a given gene in a given species (preferably at least 60% homology, more preferably at least 80%, at least 85%, at least 90%, and at least 95% homology). A method for obtaining such a species homolog is clearly understood from the description of the present specification. The term “orthologs” (also called orthologous genes) refers to genes in different species derived from a common ancestry (due to speciation). For example, in the case of the hemoglobin gene family having multigene structure, human and mouse α-hemoglobin genes are orthologs, while the human α-hemoglobin gene and the human β-hemoglobin gene are paralogs (genes arising from gene duplication). Orthologs are useful for estimation of molecular phylogenetic trees. Usually, orthologs in different species may have a function similar to that of the original species. Therefore, orthologs of the present invention may be useful in the present invention.

As used herein, the term “conservative (or conservatively modified) variant” applies to both amino acid and nucleic acid sequences. With respect to particular nucleic acid sequences, conservatively modified variants refer to those nucleic acids which encode identical or essentially identical amino acid sequences. Because of the degeneracy of the genetic code, a large number of functionally identical nucleic acids encode any given protein. For example, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” which represent one species of conservatively modified variation. Every nucleic acid sequence herein which encodes a polypeptide also describes every possible silent variation of the nucleic acid. Those skilled in the art will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence. Preferably, such modification may be performed while avoiding substitution of cysteine which is an amino acid capable of greatly affecting the higher-order structure of a polypeptide. Examples of a method for modification of a base sequence include cleavage using a restriction enzyme or the like; ligation or the like by treatment using DNA polymerase, Klenow fragments, DNA ligase, or the like; and a site specific base substitution method using synthesized oligonucleotides (specific-site directed mutagenesis; Mark Zoller and Michael Smith, Methods in Enzymology, 100, 468-500 (1983)). Modification can be performed using methods ordinarily used in the field of molecular biology.

In order to prepare functionally equivalent polypeptides, amino acid additions, deletions, or modifications can be performed in addition to amino acid substitutions. Amino acid substitution(s) refers to the replacement of at least one amino acid of an original peptide with different amino acids, such as the replacement of 1 to 10 amino acids, preferably 1 to 5 amino acids, and more preferably 1 to 3 amino acids with different amino acids. Amino acid addition(s) refers to the addition of at least one amino acid to an original peptide chain, such as the addition of 1 to 10 amino acids, preferably 1 to 5 amino acids, and more preferably 1 to 3 amino acids to an original peptide chain. Amino acid deletion(s) refers to the deletion of at least one amino acid, such as the deletion of 1 to 10 amino acids, preferably 1 to 5 amino acids, and more preferably 1 to 3 amino acids. Amino acid modification includes, but is not limited to, amidation, carboxylation, sulfation, halogenation, truncation, lipidation, alkylation, glycosylation, phosphorylation, hydroxylation, acylation (e.g., acetylation), and the like. Amino acids to be substituted or added may be naturally-occurring or nonnaturally-occurring amino acids, or amino acid analogs. Naturally-occurring amino acids are preferable.

As used herein, the term “peptide analog” or “peptide derivative” refers to a compound which is different from a peptide but has at least one chemical or biological function equivalent to the peptide. Therefore, a peptide analog includes one that has at least one amino acid analog or amino acid derivative addition or substitution with respect to the original peptide. A peptide analog has the above-described addition or substitution so that the function thereof is substantially the same as the function of the original peptide (e.g., a similar pKa value, a similar functional group, a similar binding manner to other molecules, similar water-solubility, and the like). Such a peptide analog can be prepared using techniques well known in the art. Therefore, a peptide analog may be a polymer containing an amino acid analog.

Similarly, the term “polynucleotide analog” or “nucleic acid analog” refers to a compound which is different from a polynucleotide or a nucleic acid but has at least one chemical function or biological function equivalent to that of a polynucleotide or a nucleic acid. Therefore, a polynucleotide analog or a nucleic acid analog includes one that has at least one nucleotide analog or nucleotide derivative addition or substitution with respect to the original peptide.

Nucleic acid molecules as used herein includes one in which a part of the sequence of the nucleic acid is deleted or is substituted with other base(s), or an additional nucleic acid sequence is inserted, as long as a polypeptide expressed by the nucleic acid has substantially the same activity as that of the naturally-occurring polypeptide, as described above. Alternatively, an additional nucleic acid may be linked to the 5′ terminus and/or 3′ terminus of the nucleic acid. The nucleic acid molecule may include one that is hybridizable to a gene encoding a polypeptide under stringent conditions and encodes a polypeptide having substantially the same function as that of that polypeptide. Such a gene is known in the art and can be used in the present invention.

The above-described nucleic acid can be obtained by a well-known PCR method, i.e., chemical synthesis. This method may be combined with, for example, site-specific mutagenesis, hybridization, or the like.

As used herein, the term “substitution, addition or deletion” for a polypeptide or a polynucleotide refers to the substitution, addition or deletion of an amino acid or its substitute, or a nucleotide or its substitute with respect to the original polypeptide or polynucleotide. This is achieved by techniques well known in the art, including a site-specific mutagenesis technique and the like. A polypeptide or a polynucleotide may have any number (>0) of substitutions, additions, or deletions. The number can be as large as a variant having: such a number of substitutions, additions or deletions can maintain an intended function (e.g., the information transfer function of hormones and cytokines, etc.). For example, such a number may be one or several, and preferably within 20% or 10% of the full length sequence, or no more than 100, no mote than 50, no more than 25, or the like.

(Interactive/Interaction Agent)

As used herein, the term “agent capable of specifically interacting with” a biological agent, such as a polynucleotide, a polypeptide or the like, refers to an agent which has an affinity to the biological agent, such as a polynucleotide, a polypeptide or the like, which is representatively higher than or equal to an affinity to other non-related biological agents, such as polynucleotides, polypeptides or the like (particularly, those with identity of less than 30%), and preferably significantly (e.g., statistically significantly) higher. Such an affinity can be measured with, for example, a hybridization assay, a binding assay, or the like.

As used herein, the term “agent” may refer to any substance or element as long as an intended object can be achieved (e.g., energy, etc.). Examples of such a substance include, but are not limited to, proteins, polypeptides, oligopeptides, peptides, polynucleotides, oligonucleotides, nucleotides, nucleic acids (e.g., DNA such as cDNA, genomic DNA and the like, or RNA such as mRNA, RNAi and the like), polysaccharides, oligosaccharides, lipids, low molecular weight organic molecules (e.g., hormones, ligands, information transduction substances, low molecular weight organic molecules, molecules synthesized by combinatorial chemistry, low molecular weight molecules usable as medicaments (e.g., low molecular weight molecule ligands, etc.), etc.), and composite molecules thereof. Examples of an agent specific to a polynucleotide include, but are not limited to, representatively, a polynucleotide having complementarity to the sequence of the polynucleotide with a predetermined sequence homology (e.g., 70% or more sequence identity), a polypeptide such as a transcriptional agent binding to a promoter region, and the like. Examples of an agent specific to a polypeptide include, but are not limited to, representatively, an antibody specifically directed to the polypeptide or derivatives or analogs thereof (e.g., single chain antibody), a specific ligand or receptor when the polypeptide is a receptor or ligand, a substrate when the polypeptide is an enzyme, and the like.

As used herein, the term “isolated” biological agent (e.g., nucleic acid, protein, or the like) refers to a biological agent that is substantially separated or purified from other biological agents in cells of a naturally-occurring organism (e.g., in the case of nucleic acids, agents other than nucleic acids and a nucleic acid having nucleic acid sequences other than an intended nucleic acid; and in the case of proteins, agents other than proteins and proteins having an amino acid sequence other than an intended protein). The “isolated” nucleic acids and proteins include nucleic acids and proteins purified by a standard purification method. The isolated nucleic acids and proteins also include chemically synthesized nucleic acids and proteins.

As used herein, the term “purified” biological agent (e.g., nucleic acids, proteins, and the like) refers to one from which at least a portion of naturally accompanying agents has been removed. Therefore, ordinarily, the purity of a purified biological agent is higher than that of the biological agent in a normal state (i.e., concentrated).

As used herein, the terms “purified” and “isolated” mean that the same type of biological agent is present preferably at least 75% by weight, more preferably at least 85% by weight, even more preferably at least 95% by weight, and most preferably at least 98% by weight.

(Genetic Manipulation)

When genetic manipulation is mentioned herein, the term “vector” or “recombinant vector” refers to a vector for transferring a polynucleotide sequence of interest to a target cell. Such a vector is capable of self-replication or incorporation into a chromosome in a host cell (e.g., a prokaryotic cell, yeast, an animal cell, a plant cell, an insect cell, an individual animal, and an individual plant, etc.), and contains a promoter at a site suitable for transcription of a polynucleotide of the present invention. A vector suitable for performing cloning is referred to as a “cloning vector”. Such a cloning vector ordinarily contains a multiple cloning site containing a plurality of restriction sites. Restriction enzyme sites and multiple cloning sites as described above are well known in the art and can be used as appropriate by those skilled in the art depending on the purpose in accordance with publications described herein (e.g., Sambrook et al., supra).

As used herein, the term “expression vector” refers to a nucleic acid sequence comprising a structural gene and a promoter for regulating expression thereof, and in addition, various regulatory elements in a state that allows them to operate within host cells. The regulatory element may include, preferably, terminators, selectable markers such as drug-resistance genes, and enhancers.

Examples of “recombinant vectors” for prokaryotic cells include, but are not limited to, pcDNA3(+), pBluescript-SK(+/−), pGEM-T, pEF-BOS, pEGFP, pHAT, pUC18, pFT-DEST™42GATEWAY (Invitrogen), and the like.

Examples of “recombinant vectors” for animal cells include, but are not limited to, pcDNAI/Amp, pcDNAI, pCDM8 (all commercially available from Funakoshi), pAGE107 [Japanese Laid-Open Publication No. 3-229 (Invitrogen), pAGE103 [J. Biochem., 101, 1307 (1987)], pAMo, pAMoA [J. Biol. Chem., 268, 22782-22787 (1993)], a retrovirus expression vector based on a murine stem cell virus (MSCV), pEF-BOS, pEGFP, and the like.

Examples of recombinant vectors for plant cells include, but are not limited to, pPCVICEn4HPT, pCGN1548, pCGN1549, pBI221, pBI121, and the like.

As used herein, the term “terminator” refers to a sequence which is located downstream of a protein-encoding region of a gene and which is involved in the termination of transcription when DNA is transcribed into mRNA, and the addition of a poly-A sequence. It is known that a terminator contributes to the stability of mRNA, and has an influence on the amount of gene expression.

As used herein, the term “promoter” refers to a base sequence which determines the initiation site of transcription of a gene and is a DNA region which directly regulates the frequency of transcription. Transcription is started by RNA polymerase binding to a promoter. A promoter region is usually located about 2 kbp upstream of the first exon of a putative protein coding region. Therefore, it is possible to estimate a promoter region by predicting a protein coding region in a genomic base sequence using DNA analysis software. A putative promoter region is usually located upstream of a structural gene, but this depends on the structural gene, i.e., a putative promoter region may be located downstream of a structural gene. Preferably, a putative promoter region is located within about 2 kbp upstream of the translation initiation site of the first exon.

As used herein, the term “enhancer” refers to a sequence which is used to enhance the expression efficiency of a gene of interest. One or more enhancers may be used, or no enhancer may be used.

As used herein, the term “silencer” refers to a sequence which has a function of suppressing and arresting the expression of a gene. Any silencer which has such a function may be herein used. No silencer may be used.

As used herein, the term “operably linked” indicates that a desired sequence is located such that expression (operation) thereof is under control of a transcription and translation regulatory sequence (e.g., a promoter, an enhancer, and the like) or a translation regulatory sequence. In order for a promoter to be operably linked to a gene, typically, the promoter is located immediately upstream of the gene. A promoter is not necessarily adjacent to a structural gene.

Any technique may be used herein for introduction of a nucleic acid molecule into cells, including, for example, transformation, transduction, transfection, and the like. Such a nucleic acid molecule introduction technique is well known in the art and commonly used, and is described in, for example, Ausubel F. A. et al., editors, (1988), Current Protocols in Molecular Biology, Wiley, New York, N.Y.; Sambrook J. et al. (1987) Molecular Cloning: A Laboratory Manual, 2nd Ed. and its 3rd Ed., Cold Spring Harbor Laboratory Press, Cold Spring Harbor, N.Y.; Special issue, Jikken Igaku [Experimental Medicine] “Experimental Method for Gene introduction & Expression Analysis”, Yodo-sha, 1997; and the like. Gene introduction can be confirmed by method as described herein, such as Northern blotting analysis and Western blotting analysis, or other well-known, common techniques.

Any of the above-described methods for introducing DNA into cells can be used as a vector introduction method, including, for example, transfection, transduction, transformation, and the like (e.g., a calcium phosphate method, a liposome method, a DEAE dextran method, an electroporation method, a particle gun (gene gun) method, and the like), a lipofection method, a spheroplast method (Proc. Natl. Acad. Sci. USA, 84, 1929 (1978)), a lithium acetate method (J. Bacteriol., 153, 163 (1983); and Proc. Natl. Acad. Sci. USA, 75, 1929 (1978)), and the like.

As used herein, the term “gene introduction reagent” refers to a reagent which is used in a gene introduction method so as to enhance introduction efficiency. Examples of such a gene introduction reagent include, but are not limited to, cationic polymers, cationic lipids, polyamine-based reagents, polyimine-based reagents, calcium phosphate, and the like. Specific examples of a reagent used in transfection include reagents available from various sources, such as, without limitation, Effectene Transfection Reagent (cat. no. 301425, Qiagen, CA), TransFast™ Transfection Reagent (E2431, Promega, WI), Tfx™-20 Reagent (E2391, Promega, WI), SuperFect Transfection Reagent (301305, Qiagen, CA), PolyFect Transfection Reagent (301105, Qiagen, CA), LipofectAMINE 2000 Reagent (11668-019, Invitrogen corporation, CA), JetPEI (×4) conc. (101-30, Polyplus-transfection, France) and ExGen 500 (R0511, Fermentas Inc., MD), and the like.

As used herein, “instructions” describe a method for introducing a target substance according to the present invention for users (e.g., researchers, laboratory technicians, medical doctors, patients, etc.). The instructions describe a statement indicating a method for using a composition of the present invention, or the like. The instructions are prepared in accordance with a format defined by an authority of a country in which the present invention is practiced (e.g., Health, Labor and Welfare Ministry in Japan, Food and Drug Administration (FDA) in the U.S., and the like), explicitly describing that the instructions are approved by the authority. The instructions comprise a so-called package insert in the case of medicaments or a manual in the case of experimental reagents, and are typically provided in paper media. The instructions are not so limited and may be provided in the form of electronic media (e.g., web sites, electronic mails, and the like, provided on the internet).

As used herein, the term “transformant” refers to the whole or a part of an organism, such as a cell, which is produced by transformation. Examples of a transformant include a prokaryotic cell, yeast, an animal cell, a plant cell, an insect cell, and the like. Transformants may be referred to as transformed cells, transformed tissue, transformed hosts, or the like, depending on the subject. A cell used herein may be a transformant.

When a prokaryotic cell is used herein for genetic operations or the like, the prokaryotic cell may be of, for example, genus Escherichia, genus Serratia, genus Bacillus, genus Brevibacterium, genus Corynebacterium, genus Microbacterium, genus Pseudomonas, or the like. Specifically, the prokaryotic cell is, for example, Escherichia coli XL1-Blue, Escherichia coli XL2-Blue, Escherichia coli DH1, or the like. Alternatively, a cell separated from a naturally-occurring product may be used in the present invention.

Examples of an animal cell as used herein include a mouse myeloma cell, a rat myeloma cell, a mouse hybridoma cell, a Chinese hamster ovary (CHO) cell, a baby hamster kidney (BHK) cell, an African green monkey kidney cell, a human leukemic cell, HBT5637 (Japanese Laid-Open Publication No. 63-299), a human colon cancer cell line, and the like. The mouse myeloma cell includes ps20 cells, NSO cells, and the like. The rat myeloma cell includes YB2/0 cells and the like. A human embryo kidney cell includes HEK293 cells (ATCC: CRL-1573) and the like. The human leukemic cell includes BALL-1 cells and the like. The African green monkey kidney cell includes COS-1 cells, COS-7 cells, and the like. The human colon cancer cell line includes, but is not limited to, HCT-15 cells, human neuroblastoma cells (e.g., SK-N-SH cells, SK-N-SH-5Y cells, etc.), mouse neuroblastoma cells (e.g., etc.), and the like. Alternatively, primary culture cells may be used in the present invention.

Examples of plant cells used herein in genetic manipulation include, but are not limited to, calluses or a part thereof, suspended culture cells, cells of plants in the families of Solanaceae, Poaceae, Brassicaceae, Rosaceae, Leguminosae, Cucurbitaceae, Lamiaceae, Liliaceae, Chenopodiaceae and Umbelliferae, and the like.

Gene expression (e.g., mRNA expression, polypeptide expression) may be “detected” or “quantified” by an appropriate method, including mRNA measurement and immunological measurement method. Examples of molecular biological measurement methods include Northern blotting methods, dot blotting methods, PCR methods, and the like. Examples of immunological measurement methods include ELISA methods, RIA methods, fluorescent antibody methods, Western blotting methods, immunohistological staining methods, and the like, where a microtiter plate may be used. Examples of quantification methods include ELISA methods, RIA methods, and the like. A gene analysis method using an array (e.g., a DNA array, a protein array, etc.) may be used. The DNA array is widely reviewed in Saibo-Kogaku [Cell Engineering], special issue, “DNA Microarray and Up-to-date PCR Method”, edited by Shujun-sha. Protein arrays are described in detail in Nat. Genet. 2002 December; 32 Suppl:526-32. Examples of methods for analyzing gene expression include, but are not limited to, RT-PCR methods, RACE methods, SSCP methods, immunoprecipitation methods, two-hybrid systems, in vitro translation methods, and the like, in addition to the above-described techniques. Other analysis methods are described in, for example, “Genome Analysis Experimental Method, Yusuke Nakamura's Lab-Manual, edited by Yusuke Nakamura, Yodo-sha (2002), and the like. All of the above-described publications are herein incorporated by reference.

As used herein, the term “expression” of a gene, a polynucleotide, a polypeptide, or the like, indicates that the gene or the like is affected by a predetermined action in vivo to be changed into another form. Preferably, the term “expression” indicates that genes, polynucleotides, or the like are transcribed and translated into polypeptides. In one embodiment of the present invention, genes may be transcribed into mRNA. More preferably, these polypeptides may have post-translational processing modifications.

As used herein; the term “expression level” refers to the amount of a polypeptide or mRNA expressed in a subject cell. The term “expression level” includes the level of protein expression of a polypeptide evaluated by any appropriate method using an antibody, including immunological measurement methods (e.g., an ELISA method, an RIA method, a fluorescent antibody method, a Western blotting method, an immunohistological staining method, and the like, or the mRNA level of expression of a polypeptide evaluated by any appropriate method, including molecular biological measurement methods (e.g., a Northern blotting method, a dot blotting method, a PCR method, and the like). The term “change in expression level” indicates an increase or decrease in the protein or mRNA level of expression of a polypeptide, as evaluated by an appropriate method including the above-described immunological measurement method or molecular biological measurement method.

Therefore, as used herein, the term “reduction” of “expression” of a gene, a polynucleotide, a polypeptide, or the like indicates that the level of expression is significantly reduced in the presence of or under the action of the agent of the present invention as compared to when the action of the agent is absent. Preferably, the reduction of expression includes a reduction in the amount of expression of a polypeptide. As used herein, the term “increase” of “expression” of a gene, a polynucleotide, a polypeptide, or the like indicates that the level of expression is significantly increased by introduction of an agent related to gene expression into cells (e.g., a gene to be expressed or an agent regulating such gene expression) as compared to when the action of the agent is absent. Preferably, the increase of expression includes an increase in the amount of expression of a polypeptide. As used herein, the term “induction” of “expression” of a gene indicates that the amount of expression of the gene is increased by applying a given agent to a given cell. Therefore, the induction of expression includes allowing a gene to be expressed when expression of the gene is not otherwise observed, and increasing the amount of expression of the gene when expression of the gene is observed.

As used herein, the term “specifically expressed” in relation to a gene indicates that the gene is expressed in a specific site or for a specific period of time, at a level different from (preferably higher than) that in other sites or for other periods of time. The term “specifically expressed” indicates that a gene may be expressed only in a given site (specific site) or may be expressed in other sites. Preferably, the term “specifically expressed” indicates that a gene is expressed only in a given site.

As used herein, the term “biological activity” refers to activity possessed by an agent (e.g., a polynucleotide, a protein, etc.) within an organism, including activities exhibiting various functions (e.g., transcription promoting activity, etc.). For example, when an actin acting substance interacts with actin, the biological activity thereof includes morphological changes to actin (e.g., an increase in cell extension speed, etc.) or other biological changes (e.g., reconstruction of actin filaments, etc.), and the like. Such a biological activity can be measured by, for example, visualizing actin with an actin staining reagent (Molecular Probes, Texas Red-X phalloidin) or the like, followed by microscopic inspection to observe aggregation of actin or cell extension. In another preferred embodiment, such a biological activity may be cell adhesion activity, heparin binding activity, collagen binding activity, or the like. Cell adhesion activity can be measured by, for example, measuring the rate of adhesion of disseminated cells to a solid phase, which is regarded as adhesion activity. Heparin binding activity can be measured by, for example, conducting affinity chromatography using a heparin-fixed column or the like to determine whether or not a substance binds to the column. Collagen binding activity can be measured by, for example, conducting affinity chromatography using a collagen-fixed column or the like to determine whether or not a substance binds to the column. For example, when a certain agent is an enzyme, the biological activity thereof includes enzymatic activity. In another example, when a certain agent is a ligand, the ligand binds to a corresponding receptor. Such binding activity is also a biological activity. Such biological activity can be measured using techniques well known in the art (see Molecular Cloning, Current Protocols (supra), etc.).

As used herein, the term “particle” refers to a substance which has a certain hardness and a certain size or greater. A particle used in the present invention may be made of a metal or the like. Examples of particles used in the present invention include, but are not limited to, gold colloids, silver colloids, latex colloids, and the like.

As used herein, the term “kit” refers to a unit which typically has two or more sections, at least one of which is used to provide a component (e.g., a reagent, a particle, etc.). When materials are not provided pre-mixed and are instead preferably provided such that a composition may be prepared immediately before use, a kit form is preferable. Such a kit preferably comprises instructions which describe how a component (e.g., a reagent, a particle, etc.) should be processed.

(Methods for Producing Polypeptides)

A transformant derived from a microorganism, an animal cell, or the like, which possesses a recombinant vector into which DNA encoding a polypeptide of the present invention is incorporated, is cultured according to an ordinary culture method. The polypeptide of the present invention is produced and accumulated. The polypeptide of the present invention is collected from the culture, thereby making it possible to produce the polypeptide of the present invention.

The transformant of the present invention can be cultured on a culture medium according to an ordinary method for use in culturing host cells. A culture medium for a transformant obtained from a prokaryote (e.g., E. coli) or a eukaryote (e.g., yeast) as a host may be either a naturally-occurring culture medium or a synthetic culture medium (e.g., RPMI1640 medium [The Journal of the American Medical Association, 199, 519 (1967)], Eagle's MEM medium [Science, 122, 501 (1952)], DMEM medium [Virology, 8, 396 (1959)], 199 medium [Proceedings of the Society for the Biological Medicine, 73, 1 (1950)] or these media supplemented with fetal bovine serum, or the like) as long as the medium contains a carbon source (e.g., carbohydrates (e.g., glucose, fructose, sucrose, molasses containing these, starch, starch hydrolysate, and the like), organic acids (e.g., acetic acid, propionic acid, and the like), alcohols (e.g., ethanol, propanol, and the like), etc.); a nitrogen source (e.g., ammonium salts of inorganic or organic acids (e.g., ammonia, ammonium chloride, ammonium sulfate, ammonium acetate, ammonium phosphate, and the like), and other nitrogen-containing substances (e.g., peptone, meat extract, yeast extract, corn steep liquor, casein hydrolysate, soybean cake, and soybean cake hydrolysate, various fermentation bacteria and digestion products thereof), etc.), inorganic salts (e.g., potassium (I) phosphate, potassium (II) phosphate, magnesium phosphate, magnesium sulfate, sodium chloride, ferrous sulfate, manganous sulfate, copper sulfate, calcium carbonate, etc.), and the like which an organism of the present invention can assimilate, and wherein the medium allows efficient culture of the transformant. Culture is performed under aerobic conditions for shaking culture, deep aeration agitation culture, or the like. The culture temperature is preferably 15 to 40° C., culture time is ordinarily 5 hours to 7 days. The pH of the culture medium is maintained at 3.0 to 9.0. The adjustment of pH is carried out using inorganic or organic acid, alkali solution, urea, calcium carbonate, ammonia, or the like. An antibiotic, such as ampicillin, tetracycline, or the like, may be optionally added to the culture medium during cultivation.

A polypeptide of the present invention can be isolated or purified from a culture of a transformant, which has been transformed with a nucleic acid sequence encoding the polypeptide, using an ordinary method for isolating or purifying enzymes, which are well known and commonly used in the art. For example, when a polypeptide of the present invention is secreted by a transformant for producing the polypeptide, the culture is subjected to centrifugation or the like to obtain a soluble fraction. A purified specimen can be obtained from the soluble fraction by a technique, such as solvent extraction, salting-out/desalting with ammonium sulfate or the like, precipitation with organic solvents, anion exchange chromatography with a resin (e.g., diethylaminoethyl (DEAE)-Sepharose, DIAION HPA-75 (Mitsubishi Chemical Corporation), etc.), cation exchange chromatography with a resin (e.g., S-Sepharose FF (Pharmacia), etc.), hydrophobic chromatography with a resin (e.g., buthylsepharose, phenylsepharose, etc.), gel filtration with a molecular sieve, affinity chromatography, chromatofocusing, electrophoresis (e.g., isoelectric focusing electrophoresis, etc.).

When a polypeptide of the present invention is accumulated in a soluble form within a transformant cell for producing the polypeptide, the culture is subjected to centrifugation to collect the cells in the culture. The cells are washed, followed by pulverization of the cells using a ultrasonic pulverizer, a French press, a MANTON GAULIN homogenizer, Dinomil, or the like, to obtain a cell-free extract solution. A purified specimen can be obtained from a supernatant obtained by centrifuging the cell-free extract solution or by a technique, such as solvent extraction, salting-out/desalting with ammonium sulfate or the like, precipitation with organic solvent, anion exchange chromatography with a resin (e.g., diethylaminoethyl (DEAE)-Sepharose, DIAION HPA-75 (Mitsubishi Chemical Corporation), etc.), cation exchange chromatography with a resin (e.g., S-Sepharose FF (Pharmacia), etc.), hydrophobic chromatography with a resin (e.g., buthylsepharose, phenylsepharose, etc.), gel filtration with a molecular sieve, affinity chromatography, chromatofocusing, electrophoresis (e.g., isoelectric focusing electrophoresis, etc.).

When the polypeptide of the present invention is expressed and forms insoluble bodies within cells, the cells are harvested, pulverized, and centrifuged. From the resulting precipitate fraction, the polypeptide of the present invention is collected using a commonly used method. The insoluble polypeptide is solubilized using a polypeptide denaturant. The resulting solubilized solution is diluted or dialyzed into a denaturant-free solution or a dilute solution, where the concentration of the polypeptide denaturant is too low to denature the polypeptide. The polypeptide of the present invention is allowed to form a normal three-dimensional structure, and the purified specimen is obtained by isolation and purification as described above.

Purification can be carried out in accordance with a commonly used protein purification method (J. Evan. Sadler et al.: Methods in Enzymology, 83, 458). Alternatively, the polypeptide of the present invention can be fused with other proteins to produce a fusion protein, and the fusion protein can be purified using affinity chromatography using a substance having affinity to the fusion protein (Akio Yamakawa, Experimental Medicine, 13, 469-474 (1995)). For example, in accordance with a method described in Lowe et al., Proc. Natl. Acad. Sci., USA, 86, 8227-8231 (1989), Genes Develop., 4, 1288 (1990)), a fusion protein of the polypeptide of the present invention with protein A is produced, followed by purification with affinity chromatography using immunoglobulin G.

The polypeptide of the present invention can be purified by affinity chromatography using antibodies which bind to the polypeptide. The polypeptide of the present invention can be produced using an in vitro transcription/translation system in accordance with a known method (J. Biomolecular NMR, 6, 129-134; Science, 242, 1162-1164; J. Biochem., 110, 166-168 (1991)).

Based on the amino acid information of a polypeptide as obtained above, the polypeptide can also be produced by a chemical synthesis method, such as the Fmoc method (fluorenylmethyloxycarbonyl method), the tBoc method (t-buthyloxycarbonyl method), or the like. The peptide can be chemically synthesized using a peptide synthesizer (manufactured by Advanced ChemTech, Applied Biosystems, Pharmacia Biotech, Protein Technology instrument, Synthecell-Vega, PerSeptive, Shimazu, or the like).

(Substrate/Plate/Chip/Array)

As used herein, the term “plate” refers to a planar support onto which a molecule, such as an antibody or the like, may be fixed. In the present invention, a plate preferably comprises a glass substrate (base material), which has one side provided with a thin film made of a plastic, gold, silver or aluminum.

As used herein, the term “substrate” refers to a material (preferably solid material) with which a chip or array of the present invention is constructed. Therefore, a substrate is encompassed by the concept of a plate. Examples of materials for substrates include any solid materials to which a biological molecule used in the present invention is fixed via a covalent or noncovalent bond, or which may be adapted to have such a property.

Examples of materials for plates and substrates include, but are not limited to, any material capable of forming solid surfaces, such as glass, silica, silicon, ceramics, silicon dioxide, plastics, metals (including alloys), naturally-occurring and synthetic polymers (e.g., polystyrene, cellulose, chitosan, dextran, and nylon), and the like. A substrate may be formed of a plurality of layers made of different materials. Examples of materials for plates and substrates include, but are not limited to, organic insulating materials, such as glass, quartz glass, alumina, sapphire, forsterite, silicon carbide, silicon oxide, silicon nitride, and the like. Examples of materials for plates and substrates also include, but are not limited to, organic materials, such as polyethylene, ethylene, polypropylene, polyisobutylene, polyethylene terephthalate, unsaturated polyester, fluorine-containing resin, polyvinyl chloride, polyvinylidene chloride, polyvinyl acetate, polyvinyl alcohol, polyvinyl acetal, acrylic resin, polyacrylonitrile, polystyrene, acetal resin, polycarbonate, polyamide, phenol resin, urea resin, epoxy resin, melamine resin, styrene-acrylonitrile copolymer, acrylonitrile-butadiene-styrene copolymer, silicone resin, polyphenylene oxide, polysulfone, and the like. A material preferable for a substrate varies depending on various parameters, such as measuring devices and the like, and can be selected as appropriate from the above-described various materials by those skilled in the art. For transfection arrays, a glass slide is preferable. Preferably, the base material may be coated.

As used herein, the term “coating” in relation to a solid phase support or substrate refers to an act of forming a film of a material on a surface of the solid phase support or substrate, and also refers to a film itself. Coating is performed for various purposes, such as, for example, improvement in the quality of a solid phase support and substrate (e.g., elongation of life span, improvement in resistance to hostile environment, such as resistance to acids, etc.), an improvement in affinity to a substance integrated with a solid phase support or substrate, and the like. Such a substance used for coating is herein referred to as a “coating agent”. Various materials may be used for such a coating, including, without limitation, biological substances (e.g., DNA, RNA, protein, lipid, etc.), polymers (e.g., poly-L-lysine, MAS (available from Matsunami Glass, Kishiwada, Japan), and hydrophobic fluorine resin), silane (APS (e.g., γ-aminopropyl silane, etc.)), metals (e.g., gold, etc.), in addition to the above-described solid phase support and substrate. The selection of such materials is within the technical scope of those skilled in the art and thus can be performed using techniques well known in the art. In one preferred embodiment, such a coating may be advantageously made of poly-L-lysine, silane (e.g., epoxy silane or mercaptosilane, APS (γ-aminopropyl silane), etc.), MAS, hydrophobic fluorine resin, a metal (e.g., gold, etc.). Such a material may be preferably a substance suitable for cells or objects containing cells (e.g. organisms, organs, etc.).

As used herein, the terms “chip” or “microchip” are used interchangeably to refer to a micro-integrated circuit which has versatile functions and constitutes a portion of a system. Examples of a chip include, but are not limited to, DNA chips, protein chips, and the like.

As used herein, the terms “array” and “bioassay” are used interchangeably to refer to a substrate (e.g., a chip, etc.) which has a pattern of a composition containing at least one (e.g., 1000 or more, etc.) target substances (e.g., DNA, proteins, transfection mixtures, etc.), which are arrayed. Among arrays, patterned substrates having a small size (e.g., 10×10 mm, etc.) are particularly referred to as microarrays. The terms “microarray” and “array” are used interchangeably. Therefore, a patterned substrate having a larger size than that which is described above may be referred to as a microarray. For example, an array comprises a set of desired transfection mixtures fixed to a solid phase surface or a film thereof. An array preferably comprises at least 10² antibodies of the same or different types, more preferably at least 10³, even more preferably at least 10⁴, and still even more preferably at least 10⁵. These antibodies are placed on a surface of up to 125×80 mm, more preferably 10×10 mm. An array includes, but is not limited to, a 96-well microtiter plate, a 384-well microtiter plate, a microtiter plate the size of a glass slide, and the like. A composition to be fixed may contain one or a plurality of types of target substances. Such a number of target substance types may be in the range of from one to the number of spots, including, without limitation, about 10, about 100, about 500, and about 1,000.

As described above, any number of target substances (e.g., proteins, such as antibodies) may be provided on a solid phase surface or film, typically including no more than 10⁸ biological molecules per substrate, in another embodiment no more than 10⁷ biological molecules, no more than 10⁶ biological molecules, no more than 10⁵ biological molecules, no more than 10⁴ biological molecules, no more than 10³ biological molecules, or no more than 10² biological molecules. A composition containing more than 10⁸ biological molecule target substances may be provided on a substrate. In these cases, the size of a substrate is preferably small. Particularly, the size of a spot of a composition containing target substances (e.g., proteins such as antibodies) may be as small as the size of a single biological molecule (e.g., 1 to 2 nm order). In some cases, the minimum area of a substrate may be determined based on the number of biological molecules on a substrate. A composition containing target substances, which are intended to be introduced into cells, are herein typically arrayed on and fixed via covalent bonds or physical interaction to a substrate in the form of spots having a size of 0.01 mm to 10 mm.

“Spots” of biological molecules may be provided on an array. As used herein, the term “spot” refers to a certain set of compositions containing target substances. As used herein, the term “spotting” refers to an act of preparing a spot of a composition containing a certain target substance on a substrate or plate. Spotting may be performed by any method, for example, pipetting or the like, or alternatively, using an automatic device. These methods are well known in the art.

As used herein, the term “address” refers to a unique position on a substrate, which may be distinguished from other unique positions. Addresses are appropriately associated with spots. Addresses can have any distinguishable shape such that substances at each address may be distinguished from substances at other addresses (e.g., optically). A shape defining an address may be, for example, without limitation, a circle, an ellipse, a square, a rectangle, or an irregular shape. Therefore, the term “address” is used to indicate an abstract concept, while the term “spot” is used to indicate a specific concept. Unless it is necessary to distinguish them from each other, the terms “address” and “spot” may be herein used interchangeably.

The size of each address particularly depends on the size of the substrate, the number of addresses on the substrate, the amount of a composition containing target substances and/or available reagents, the size of microparticles, and the level of resolution required for any method used with the array. The size of each address may be, for example, in the range of from 1-2 nm to several centimeters, though the address may have any size suited to an array.

The spatial arrangement and shape which define an address are designed so that the microarray is suited to a particular application. Addresses may be densely arranged or sparsely distributed, or subgrouped into a desired pattern appropriate for a particular type of material to be analyzed.

Microarrays are widely reviewed in, for example, “Genomu Kino Kenkyu Purotokoru [Genomic Function Research Protocol] (Jikken Igaku Bessatsu [Special Issue of Experimental Medicine], Posuto Genomu Jidai no Jikken Koza 1 [Lecture 1 on Experimentation in Post-genome Era), “Genomu Ikagaku to korekarano Genomu Iryo [Genome Medical Science and Futuristic Genome Therapy (Jikken Igaku Zokan [Special Issue of Experimental Medicine]), and the like.

A vast amount of data can be obtained from a microarray. Therefore, data analysis software is important for administration of correspondence between clones and spots, data analysis, and the like. Such software may be attached to various detection systems (e.g., Ermolaeva O. et al., (1998) Nat. Genet., 20: 19-23). The format of database includes, for example, GATC (genetic analysis technology consortium) proposed by Affymetrix.

Micromachining for arrays is described in, for example, Campbell, S. A. (1996), “The Science and Engineering of Microelectronic Fabrication”, Oxford University Press; Zaut, P. V. (1996), “Microarray Fabrication: a Practical Guide to Semiconductor Processing”, Semiconductor Services; Madou, M. J. (1997), “Fundamentals of Microfabrication”, CRC15 Press; Rai-Choudhury, P. (1997), “Handbook of Microlithography, Micromachining, & Microfabrication: Microlithography”; and the like, portions related thereto of which are herein incorporated by reference.

(Cells)

The term “cell” is herein used in its broadest sense in the art, referring to a structural unit of tissue of a multicellular organism, which is capable of self replicating, has genetic information and a mechanism for expressing it, and is surrounded by a membrane structure which isolates the living body from the outside. Cells used herein may be either naturally-occurring cells or artificially modified cells (e.g., fusion cells, genetically modified cells, etc.). Examples of cell sources include, but are not limited to, a single-cell culture; the embryo, blood, or body tissue of normally-grown transgenic animal; a cell mixture of cells derived from normally-grown cell lines; and the like.

Cells used herein may be derived from any organism (e.g., any unicellular organisms (e.g., bacteria and yeast) or any multicellular organism (e.g., animals (e.g., vertebrates and invertebrates), plants (e.g., monocotyledons and dicotyledons, etc.)). For example, cells used herein are derived from a vertebrate (e.g., Myxiniformes, Petronyzoniformes, Chondrichthyes, Osteichthyes, amphibian, reptilian, avian, mammalian, etc.), more preferably mammalian (e.g., monotremata, marsupialia, edentate, dermoptera, chiroptera, carnivore, insectivore, prbboscidea, perissodactyla, artiodactyla, tubulidentata, pholidota, sirenia, cetacean, primates, rodentia, lagomorpha, etc.). In one embodiment, cells derived from Primates (e.g. chimpanzee, Japanese monkey, human) are used. Particularly, without limitation, cells derived from a human are used.

As used herein, the term “stem cell” refers to a cell capable of self replication and pluripotency. Typically, stem cells can regenerate an injured tissue. Stem cells used herein may be, but are not limited to, embryonic stem (ES) cells or tissue stem cells (also called tissular stem cells, tissue-specific stem cells, or somatic stem cell). A stem cell may be an artificially produced cell (e.g., fusion cells, reprogrammed cells, or the like used herein) as long as it has the above-described abilities. Embryonic stem cells are pluripotent stem cells derived from early embryos. An embryonic stem cell was first established in 1981, which has been applied to production of knockout mice since 1989. In 1998, a human embryonic stem cell was established, which is currently becoming available for regenerative medicine. Tissue stem cells have a relatively limited level of differentiation, unlike embryonic stem cells. Tissue stem cells are present in tissues and have an undifferentiated intracellular structure. Tissue stem cells have a higher nucleus/cytoplasm ratio and have few intracellular organelles. Most tissue stem cells have pluripotency, a long cell cycle, and proliferative ability beyond the life of the individual. As used herein, stem cells may be preferably embryonic stem cells, though tissue stem cells may also be employed, depending on the circumstance.

Tissue stem cells are separated into categories of sites from which the cells are derived, such as the dermal system, the digestive system, the bone marrow system, the nervous system, and the like. Tissue stem cells in the dermal system include epidermal stem cells, hair follicle stem cells, and the like. Tissue stem cells in the digestive system include pancreatic (common) stem cells, liver stem cells, and the like. Tissue stem cells in the bone marrow system include hematopoietic stem cells, mesenchymal stem cells, and the like. Tissue stem cells in the nervous system include neural stem cells, retinal stem cells, and the like.

As used herein, the term “somatic cell” refers to any cell other than a germ cell, such as an egg, a sperm, or the like, which does not transfer its DNA to the next generation. Typically, somatic cells have limited or no pluripotency. Somatic cells used herein may be naturally-occurring or genetically modified as long as they can achieve the intended treatment.

The origin of a stem cell is categorized into the ectoderm, endoderm, or mesoderm. Stem cells of ectodermal origin are mostly present in the brain, including neural stem cells. Stem cells of endodermal origin are mostly present in bone marrow, including blood vessel stem cells, hematopoietic stem cells, mesenchymal stem cells, and the like. Stem cells of mesoderm origin are mostly present in organs, including liver stem cells, pancreas stem cells, and the like. Somatic cells may be herein derived from any germ layer. Preferably, somatic cells, such as lymphocytes, spleen cells or testis-derived cells, may be used.

As used herein, the term “isolated” means that naturally accompanying material is at least reduced, or preferably substantially completely eliminated, in normal circumstances. Therefore, the term “isolated cell” refers to a cell substantially free from other accompanying substances (e.g., other cells, proteins, nucleic acids, etc.) in natural circumstances. The term “isolated” in relation to nucleic acids or polypeptides means that, for example, the nucleic acids or the polypeptides are substantially free from cellular substances or culture media when they are produced by recombinant DNA techniques; or precursory chemical substances or other chemical substances when they are chemically synthesized. Isolated nucleic acids are preferably free from sequences naturally flanking the nucleic acid within an organism from which the nucleic acid is derived (i.e., sequences positioned at the 5′ terminus and the 3′ terminus of the nucleic acid).

As used herein, the term “established” in relation to cells refers to a state of a cell in which a particular property (pluripotency) of the cell is maintained and the cell undergoes stable proliferation under culture conditions. Therefore, established stem cells maintain pluripotency.

As used herein, the term “differentiated cell” refers to a cell having a specialized function and form (e.g., muscle cells, neurons, etc.). Unlike stem cells, differentiated cells have no or little pluripotency. Examples of differentiated cells include epidermal cells, pancreatic parenchymal cells, pancreatic duct cells, hepatic cells, blood cells, cardiac muscle cells, skeletal muscle cells, osteoblasts, skeletal myoblasts, neurons, vascular endothelial cells, pigment cells, smooth muscle cells, fat cells, bone cells, cartilage cells, and the like.

(Medicaments and Cosmetics, and Therapy and Prevention Using the Same)

In another aspect, the present invention relates to medicaments (e.g., pharmaceuticals or medicaments (vaccine, etc.), health foods, pharmaceuticals or medicaments comprising a protein or lipid having reduced antigenicity, etc.), cosmetics, agricultural chemicals, foods, and the like, for introducing an effective ingredient into cells. Such medicaments and cosmetics may further comprise a pharmaceutically acceptable carrier. Such a pharmaceutically acceptable carrier contained in a medicament of the present invention includes any known substance.

Examples of a pharmaceutically acceptable carrier or a suitable formulation material include, but are not limited to, antioxidants, preservatives, colorants, flavoring agents, diluents, emulsifiers, suspending agents, solvents, fillers, bulking agents, buffers, delivery vehicles, and/or pharmaceutical adjuvants. Representatively, a medicament of the present invention is administered in the form of a composition comprising a compound, or a variant or derivative thereof, with at least one physiologically acceptable carrier, excipient or diluent. For example, an appropriate vehicle may be injection solution, physiological solution, or artificial cerebrospinal fluid, which can be supplemented with other substances which are commonly used for compositions for parenteral delivery.

Acceptable carriers, excipients or stabilizers used herein preferably are non-toxic to recipients and are preferably inert at the dosages and concentrations employed, and preferably include phosphate, citrate, or other organic acids; ascorbic acid, α-tocopherol; low molecular weight polypeptides; proteins (e.g., serum albumin, gelatin, or immunoglobulins); hydrophilic polymers (e.g., polyvinylpyrrolidone); amino acids (e.g., glycine, glutamine, asparagine, arginine or lysine); monosaccharides, disaccharides, and other carbohydrates (glucose, mannose, or dextrins); chelating agents (e.g., EDTA); sugar alcohols (e.g., mannitol or sorbitol); salt-forming counterions (e.g., sodium); and/or nonionic surfactants (e.g., Tween, pluronics or polyethylene glycol (PEG)).

Examples of appropriate carriers include neutral buffered saline or saline mixed with serum albumin. Preferably, the product is formulated as a lyophilizate using appropriate excipients (e.g., sucrose). Other standard carriers, diluents, and excipients may be included as desired. Other exemplary compositions comprise Tris buffer of about pH 7.0-8.5, or acetate buffer of about pH 4.0-5.5, which may further include sorbitol or a suitable substitute therefor.

The medicament of the present invention may be administered orally or parenterally. Alternatively, the medicament of the present invention may be administered intravenously or subcutaneously. When systemically administered, the medicament for use in the present invention may be in the form of a pyrogen-free, pharmaceutically acceptable aqueous solution. The preparation of such pharmaceutically acceptable compositions, with due regard to pH, isotonicity, stability and the like, is within the skill of the art. Administration methods may be herein oral, parenteral administration (e.g., intravenous, intramuscular, subcutaneous, intradermal, to mucosa, intrarectal, vaginal, topical to an affected site, to the skin, etc.). A prescription for such administration may be provided in any formulation form. Such a formulation form includes liquid formulations, injections, sustained release preparations, and the like.

The medicament of the present invention may be prepared for storage by mixing a sugar chain composition having the desired degree of purity with optional physiologically acceptable carriers, excipients, or stabilizers (Japanese Pharmacopeia ver. 14, or a supplement thereto or the latest version; Remington's Pharmaceutical Sciences, 18th Edition, A. R. Gennaro, ed., Mack Publishing Company, 1990; and the like), in the form of lyophilized cake or aqueous solution.

The amount of the composition of the present invention used in the treatment method of the present invention can be easily determined by those skilled in the art with reference to the purpose of use, a target disease (type, severity, and the like), the patient's age, weight, sex, and case history, the form or type of the cell, and the like. The frequency of the treatment method of the present invention applied to a subject (or patient) is also determined by those skilled in the art with respect to the purpose of use, target disease (type, severity, and the like), the patient's age, weight, sex, and case history, the progression of the therapy, and the like. Examples of the frequency include once per day to several months (e.g., once per week to once per month). Preferably, administration is performed once per week to month with reference to the progression.

When the present invention is used for other applications, such as cosmetics, food, agricultural chemicals, and the like, it may be prepared in accordance with limitations defined by the authority.

### DESCRIPTION OF PREFERRED EMBODIMENTS

Hereinafter, the present invention will be described by way of embodiments. The embodiments described below are provided only for illustrative purposes. Accordingly, the scope of the present invention is not limited by the embodiments except as by the appended claims.

In one aspect, the present invention provides a composition for increasing the efficiency of introducing a target substance into a cell, comprising a cellular adhesion related agent. The above-described object of the present invention was achieved by unexpectedly finding that the introduction of a substance (e.g., DNA, RNA, polypeptides, sugar chains or a composite substance thereof, etc.), which is not substantially introduced under normal conventional conditions, is promoted by the action of a cellular adhesion related agent (typically, an antibody to an integrin receptor). Particularly, it was found that such a cellular adhesion related agent has the significant effect of promoting introduction efficiency in genetic manipulation using DNA, such as transfection. Such a finding has not been conventionally known or expected. Attention should be focused onto the present invention which represents a significant breakthrough in gene research.

In a preferable embodiment, the cellular adhesion related agent used in the present invention comprises an interaction substance interacting with a cellular adhesion molecule such as an extracellular matrix molecule, integrin receptor, RGD molecules and the like.

In a preferable embodiment, the interaction substance used in the present invention causes an antigen-antibody reaction with a partner of a cellular adhesion molecule. Accordingly, the interaction substances of the present invention may be an antibody (for example, monoclonal antibodies, polyclonal antibodies, and the like), or derivatives thereof (chimeric antibodies, antibody fragments and the like).

Preferably, the interaction substance comprises an antibody selected from the group consisting of an anti-CD49a antibody, an anti-CD49b antibody, an anti-CD49c antibody, an anti-CD49d antibody, an anti-CD49e antibody, and an anti-CD49f antibody. Such antibodies are preferable, since these antibodies unexpectedly enhance the intracellular introduction efficiency of a genetic material (DNA). Although not wishing to be bound by any theory, such an effect is preferable, as adhesion of a cell using substrates such as extracellular matrix molecules essentially means cellular adhesion via integrin including CD49a-f. Such adhesion via cellular adhesion related agents attains preferable effects on a cell, i.e., in this instance in particular, enhancement of intracellular introduction efficiency of a genetic material (DNA) is observed (see the applicants' previous patents/patent applications or references cited therein). Accordingly, use of an anti-CD49 antibody as an adhesion substrate means that it is possible to predict that the action is mimicked with respect to the functions thereof. Accordingly, an antibody against an anti-integrin molecule, including CD49a-f relating to CD49, may also be included in the preferable embodiments of the present invention.

In an embodiment, the target substance to be targeted in the present invention includes a genetic material, for example, nucleic acids such as DNA, RNA and the like. Such DNA includes, but is not limited to, for example, plasmid DNA, naked DNA, cDNA, genomic DNA and the like.

In one embodiment, the integrin receptors intended in the present invention may be selected from the group consisting of CD49a, CD49b, CD49c, CD49d, CD49e, CD49f, and CD29 (for example, C29, CD49a, CD49c, CD49d, CD49e and CD49f). Although not wishing to be bound by any theory, CD29 is also preferable, because adhesion of a cell using substrates such as extracellular matrix molecules essentially means cellular adhesion via integrin. CD29 is B subunit of an integrin receptor, and is a common unit for many integrin receptors. Such adhesion via cellular adhesion related agents attains preferable effects on a cell, i.e., in this instance in particular, enhancement of intracellular introduction efficiency of a genetic material (DNA) is observed (see the applicants' previous patents/patent applications or references cited therein). Accordingly, use of an anti-CD29 antibody as an adhesion substrate means that it is possible to predict that the action is mimicked with respect to the functions thereof. Accordingly, an antibody against an anti-integrin molecule, including CD29, may also be included in the preferable embodiments of the present invention.

Alternatively, integrin receptors may be those interacting with a molecule selected from the group consisting of collagen, fibronectin, vitronectin and laminin.

In one embodiment, the cell targeted by the present invention may comprise at least one cell selected from the group consisting of a stem cell and a differentiated cell. In the present invention, the target of the present invention may be either a stem cell or a differentiated cell. Depending on the type of a cell used, the agent relating to cellular adhesion may differ. As such, it is preferable to select a cellular adhesion related agent depending on the intended cell. Preferably, a cellular adhesion molecule is specifically expressed in a cell. Any known molecule in the art may be used as such a cell specific adhesion molecule, or alternatively, those skilled in the art may identify such a molecule by means of any well-known technology in the art.

In a preferable embodiment, the target substance used in the present invention is a genetic material, and preferably it is advantageous to further include a gene introduction reagent. The genetic material used herein may be any genetic material suitable for a gene introduction reagent used, and is usually DNA.

Accordingly, in an embodiment in which gene introduction is contemplated, the composition of the present invention preferably further comprises a gene introduction reagent. Inclusion of such a gene introduction reagent allows synergistic effects of introduction enhancement according to the present invention.

The gene introduction reagents used in the present invention may be selected from the group consisting of a cationic macromolecule, a cationic lipid and calcium phosphate. Such gene introduction reagents include but are not limited to, Effectene, TransFast™, Tfx™-20, SuperFect, PolyFect, LipofectAMINE 2000, JetPEI and ExGen 500 and the like. LipofectAMINE 2000 is preferable as the gene introduction efficiency thereof is high.

In another embodiment, the composition of the present invention further comprises a particle. The inclusion of a particle allows introduction of a material into a cell, in particular, efficient targeted introduction thereof. Examples of such a particle include, but are not limited to, for example, a metal colloid such as a gold colloid.

In another preferable embodiment, the composition of the present invention further comprises a salt. Although not wishing to be bound by any theory, it is believed that such inclusion of a salt enhances immobilization effects in the case of using a support, or alternatively, enhances the effects wherein the three-dimensional structure of a target substance is retained in a more appropriate form.

Such a salt used in the present invention may be any salt, such as an inorganic salt or an organic salt, and a mixture of a plurality of salts is preferably used in comparison to a simple salt. The mixtures of a plurality of salts include, but are not limited to, for example, salts included in buffers or cell-culture media or the like.

In a preferable embodiment, the cellular adhesion related agent included in the composition of the present invention is an antibody against an integrin receptor or a variant or fragment thereof. In the present invention, it was found that an antibody against an integrin receptor or a variant or fragment thereof unexpectedly attains targeted substance introduction effects. As such, the present invention should be of note for the effects of enhanced introduction efficiency of a material into a cell by means of an extracellular matrix protein.

Polypeptides intended in the present invention such as integrins and the like, may be a polypeptide having an amino acid sequence known in the art or a variant sequence thereof, and has a biological activity.

Alternatively, in a preferable embodiment, the number of the substitutions, additions and deletions in the variant sequence is limited to, for example, 50 or less, 40 or less, 30 or less, 30 or less, 20 or less, 15 or less, 10 or less, 9 or less, 8 or less, 7 or less, 6 or less, 5 or less, 4 or less, 3 or less, 2 or less. In a particular embodiment, the number of such substitutions, additions and/or deletions may be one or more or several. The less the number of the substitution, addition and deletion is, the more preferable it is. However, as long as the biological activity is retained (preferably, having an activity similar to or substantially identical to that of cellular adhesion related agent), the number thereof may be large.

In another preferable embodiment, an allelic gene variant of the polypeptide having at least 90% homology to the sequence on which the comparison is based, may be used. Within the same lineage, it is preferable that such an allelic gene variant has at least 99% homology.

The species homologues may be identified, when there is a gene sequence database for the species, by searching the database using the entirety or a portion of the gene sequence of the extracellular matrix protein, or integrin receptor of the present invention (for example, CD49 family) as a query. Alternatively, an entire or a portion of the gene of the present invention may be used as a probe or a primer so as to allow the identification of the homologues by screening the genetic library of the subject species. Such an identification method is well known in the art, and is described in the references cited herein. Such a method of identification was well known in the art, and is also described in the art cited herein. Species homologues preferably have for example at least about 30% homology with the original sequence thereof. Species homologues more preferably have at least about 50% with the original sequence thereof.

The concentration of the cellular adhesion related agents may be readily determined by those skilled in the art in view of the description of the present specification. For example, examples of such concentrations may be at least about 0.1 μg/μL, preferably about 0.2 μg/μL, more preferably 0.5 μg/μL. In one embodiment, concentrations exceeding about 0.5 μg/μL reaches plateau with respect to increased activity, and thus the concentration of about 0.5 μg/μL to 2.0 μg/μL may be a preferable concentration range.

(Kits)

In another aspect, the present invention provides a kit for enhancing gene introduction efficiency. The present kit comprises (a) a cellular adhesion related agent; and (b) a gene introduction reagent. As a cellular adhesion related molecule, any embodiment described hereinabove in detail may be used in the composition for enhancing the efficiency of the introduction of a target substance according to the present invention. Such an embodiment may be selected and practiced in an appropriate format based on the description of the present specification, by those skilled in the art. When the present invention is provided in the form of a kit, such a kit may further comprise an instruction. Such an instruction is prepared according to the format defined by the controlling authority of a country of interest where the present invention is practiced, and the instruction may expressly describe that the kit has received authorization from the controlling authority but the present invention is not limited thereto. An instruction is provided as a manual, usually in a paper format, but is not limited thereto, and may be provided, for example, in an embodiment in a form of an electronic medium (for example, a website provided via the Internet, or an electronic mail). As such a cellular adhesion related agent, as described above, any embodiment applied to the composition for enhancing the efficiency of the introduction of a target substance into a cell according to the present invention by means of appropriate selection by those skilled in the art. Accordingly, preferably, cellular adhesion related agents may be an antibody to an integrin receptor or a variant or fragment thereof. More preferably, an antibody selected from the group consisting of an anti-CD49a antibody, an anti-CD49b antibody, an anti-CD49c antibody, an anti-CD49d antibody, an anti-CD49e antibody, and an anti-CD49f antibody, or a fragment or a variant thereof may be used in the present invention.

(Compositions)

In another aspect, the present invention provides a composition for enhancing the introduction efficiency of a target substance into a cell. The present invention was achieved by unexpectedly finding that the introduction of a substance (e.g., DNA, RNA, polypeptides, sugar chains or a composite substance thereof, etc.), which is not substantially introduced under normal conventional conditions, is promoted by the activity of a cellular adhesion related agent (typically, an antibody to an integrin receptor). In this case, the present invention is provided in the format of a composition comprising a target substance and a cellular adhesion related agent. The present invention may be practiced by means of an appropriate selection by those skilled in the art for the embodiment applied to the composition for enhancing the efficiency of introduction of a target substance into a cell according to the present invention. Accordingly, preferably, the cellular adhesion related agent may be an anti-integrin receptor antibody or a variant thereof. More preferably, an antibody selected from the group consisting of an anti-CD49a antibody, an anti-CD49b antibody, an anti-CD49c antibody, an anti-CD49d antibody, an anti-CD49e antibody, and an anti-CD49f antibody, or a fragment or a variant thereof may be used in the present invention.

Targeted substances included in the composition for use in introduction of a targeted substance into a cell according to the present invention, preferably include but are not limited to, for example, DNA, RNA, polypeptide, sugar and a complex thereof and the like. In a particular preferable embodiment, DNA is selected as a targeted substance. Such DNA preferably encodes a gene of interest in the case of directing gene expression. Accordingly, in an embodiment of directing transfection, a targeted substance comprises a DNA encoding a gene sequence to be transfected. In another preferable embodiment, RNA is selected as a targeted substance. Such an RNA preferably encodes a gene of interest when directing expression of the gene. In this case, RNA encoding a gene sequence with a gene introduction agent suitable for an RNA is preferably used.

In an embodiment of directing the introduction of a gene, the composition for use in introduction of a targeted substance into a cell according to the present invention, may further comprise a gene introduction reagent. Although not wishing bound by any theory, in one embodiment, it is believed that such a gene introduction reagent and the cellular adhesion related agent found in the present invention cooperatively act to introduce a gene into a cell in an efficient manner which has never achieved by means of conventional technology.

In a preferable embodiment, such a gene introduction reagent which may be included in the composition of the present invention, includes but is not limited to, for example, cationic macromolecules, cationic lipids, polyamine reagents, polyimine reagents, calcium phosphate and the like.

In a preferable embodiment, a composition for use in introduction of a targeted substance into a cell according to the present invention may be present as a liquid phase. When it exists as a liquid phase, the present invention is useful as a liquid transfection system, for example.

In another preferable embodiment, the composition for use in introduction of a targeted substance into a cell according to the present invention may be present as a solid phase. When it exists as a solid phase, the present invention is useful as a solid transfection system, for example. Preferable embodiments of such a solid transfection include, but are not limited to, for example, transfection using a microtitreplate or an array (or a chip). When introducing a polypeptide, such liquid and solid phases are useful formats.

(Devices)

In another aspect, the present invention further provides a device for enhancing gene introduction efficiency of a target molecule into a cell. The device comprises (a) a target molecule; and (b) a cellular adhesion related agent, wherein the cellular adhesion related agent is immobilized on to a support. The device of the present invention has been achieved due to the unexpected discovery of the enhancement of introduction of a target substance, which is rarely introduced into a cell under conventional conditions (for example, DNA, RNA, polypeptide, sugar or the complex substance thereof), by means of the action of a cellular adhesion related agent (typically, an antibody to an integrin receptor or a variant thereof). In this case, the present invention is provided in a form in which a composition comprising a target substance and a cellular adhesion related agent is immobilized onto a solid support. Such a cellular adhesion related agent allows the present invention to be performed by appropriately selecting a format to be applied to a composition for enhancing the introduction efficiency of a targeted substance into a cell according to the present invention, by those skilled in the art. Accordingly, in a preferable embodiment, the cellular adhesion related agents may be an antibody against an integrin receptor or a variant thereof. More preferably, an antibody selected from the group consisting of an anti-CD49a antibody, an anti-CD49b antibody, an anti-CD49c antibody, an anti-CD49e antibody and an anti-CD49f antibody, and fragments or variants thereof may be used herein.

The target substance to be comprised in the device of the present invention preferably includes, but is not limited to, for example, DNA, RNA, polypeptide, sugar and a complex thereof, and the like. In a particular preferable embodiment, DNA is selected as the target substance. Such DNA preferably encodes a gene sequence for the purpose of gene expression when it is an object of the invention to express the gene. Accordingly, in an embodiment directed to transfection, the target substance comprises a DNA encoding a gene sequence to be transfected.

In an embodiment directed to the introduction of a gene, the device of the present invention may further comprise a gene introduction reagent. Although not wishing to be bound to any theory, in one embodiment, it is believed that such a gene introduction reagent and the cellular adhesion related agent found in the present invention act in combination to achieve introduction of a gene into a cell in an efficient manner which has not been conventionally achieved. In the present device a composition is immobilized onto a solid support, and it is preferable that gene introduction reagents having compatibility to a solid support are used.

In a preferable embodiment, the solid support used in the device of the present invention may be selected from the group consisting of a plate, micro-well plate, chip, glass slide, film, bead and metal.

In a particular embodiment, in the case where a chip is used as a solid support in the device of the present invention, such a device may also be called an array. Arrays usually comprise biomolecules (for example, DNA, proteins and the like) intended for introduction, positioned on a support in a arrayed or patterned manner. Such an array for use in transfection is called herein as a transfection array. In the present invention, it was elucidate that stem cells or the like, which had not been possible to transfect by means of conventional systems may now be transfected. Accordingly, compositions, devices, and methods using a cellular adhesion related agent according to the present invention has achieved unexpectedly significant effects, which had not been achieved by means of prior art technology, by providing a transfection array applicable to any cell.

As used herein, the solid support used in the device of the present invention preferably has a coating. Coating enhances the quality of a solid support and a substrate (for example, improvement in shelf-life, or improvement in environmental friendliness, such as anti-acidity and the like), improvement in affinity to a substance to be bound to a solid support or a substrate, and to a cell. In a preferable embodiment, such a coating comprises a coating agent such as poly-L-lysine, silane such as APS (gamma-aminopropyl silane), MAS, hydrophobic fluorine resin, silanes such as epoxy silane or mercapto silane, and metal such as gold and the like. Preferably, the coating agent is poly-L-lysine.

(A Method for Enhancing Introduction Efficiency into a Cell)

In another aspect, the present invention provides a method for enhancing introduction efficiency of a target substance into a cell. The present method is accomplished by the discovery that when a target substance (for example, DNA, RNA, polypeptide, sugar chain or a complex thereof) which are rarely introduced into a cell under conventional conditions, are presented, preferably contacted, to a cell with a cellular adhesion related agent, introduction of the target substance into a cell in an efficient manner is achieved for the first time. Accordingly, the present invention comprises the steps of A) providing a target substance; and B) providing a cellular adhesion related agent, in an undetermined order. The present method may further comprise the step of C) contacting the target substance and the cellular adhesion related substance with the cell. A target substance and a cellular adhesion related agent may be provided in combination or in a separate manner. As a cellular adhesion related molecule, any embodiment described hereinabove in detail may be used in the composition for enhancing the efficiency of the introduction of a target substance according to the present invention as described above. Such an embodiment may be selected and practiced in an appropriate format based on the description of the present specification. Thus, such a cellular adhesion related agent allows the present invention to be performed by appropriately selecting a format to be applied to a composition for enhancing the introduction efficiency of a targeted substance into a cell according to the present invention, by those skilled in the art. Accordingly, in a preferable embodiment, the cellular adhesion related agents may be an antibody against an integrin receptor or a variant thereof. More preferably, an antibody selected from the group consisting of an anti-CD49a antibody, an anti-CD49b antibody, an anti-CD49c antibody, an anti-CD49e antibody and an anti-CD49f antibody, and fragments or variants thereof may be used herein.

The target substances used in a method according to the present invention include, but are not limited to, preferably e.g., DNA, RNA, polypeptide, sugar and a complex thereof.

In a particularly preferable embodiment, DNA is selected as a target substance. When such a DNA is directed to gene expression, it preferably encodes a gene of interest. Accordingly, in an embodiment directed to transfection, the target substance comprises a DNA encoding the gene sequence to be transfected.

In an embodiment directed to the introduction of a gene, the methods according to the present invention may further use a gene introduction reagent. Although not wishing to be bound by any theory, in an embodiment, it is believed that such a gene introduction reagent and a cellular adhesion related agent found in the present invention synergistically act to achieve efficient gene introduction into a cell which has not conventionally achieved. The provision of gene introduction reagent may be in combination or provided separately with a target substance and/or a cellular adhesion related agent. Preferably, it may be advantageous that provision of a cellular adhesion related agent is provided after the formation of a complex between a target substance and a gene introduction reagent. Although not wishing to be bound by any theory, such an order appears to enhance the introduction efficiency.

In a preferable embodiment, the gene introduction reagents used in the present invention include but are not limited to a cationic macromolecule, a cationic lipid, a polyamine reagent, a polyimine reagent, calcium phosphate and the like.

A cell of interest according to the present invention may include any cell as long as the introduction of a target substance thereinto is intended, and includes, for example, stem cells, somatic cells and the like. Significant effects according to the present invention includes achieving the introduction of a target substance using a method such as transfection in a universal manner for any cells regardless of the type of cells such as stem cells, somatic cells and the like, which is an effect not achieved by the conventional methods. Preferably, the target includes a tissue stem cell but is not limited thereto, and may include an embryonic stem cell. Although not wishing bound by any theory, tissue stem cells appear to have better introduction efficiency than embryonic stem cells, amongst the stem cells.

In a particular embodiment, the method for introducing a target substance into a cell according to the present invention, the method may partially or entirely be conducted in a liquid phase. In another particular embodiment, the method may partially or entirely be conducted in a solid phase. Accordingly, the method may also be conducted in combination of a liquid phase and a solid phase.

(A Method for Enhancing Introduction Efficiency into a Cell on a Support)

In another aspect, the present invention provides a method for enhancing the efficiency of the introduction of a target substance into a cell using a support. The present invention has been accomplished by the discovery that when a target substance (for example, DNA, RNA, polypeptide, sugar chain or the complex substance thereof), which are rarely introduced into a cell under the conventional conditions, are presented, preferably contacted, to a cell with a cellular adhesion related agent, introduction of the target substance into a cell in an efficient manner is achieved for the first time. The effects of enhancing the efficiency of the introduction of a target substance (in particular, DNA, preferably DNA comprising a sequence encoding a gene to be transfected) by means of a support, could not been achieved or had not even been expected in the conventional art. Therefore, such an effect can be said to represent a significant breakthrough in the art. Accordingly, the method using a solid support according to the present invention comprises the steps of: I) immobilizing, to a support, a composition comprising A) a target substance; and B) a cellular adhesion related agent; and II) contacting a cell with the composition on the support. As a cellular adhesion related molecule, any embodiment described hereinabove in detail may be used in the composition for enhancing the efficiency of the introduction of a target substance according to the present invention, as described above. Such an embodiment may be selected and practiced in an appropriate format based on the description of the present specification, by those skilled in the art. As such a cellular adhesion related agent, as described above, any embodiment applied to the composition for enhancing the efficiency of the introduction of a target substance into a cell according to the present invention by means of appropriate selection by those skilled in the art. Accordingly, preferably, cellular adhesion related agents may be an antibody against an integrin receptor or a variant or fragment thereof. More preferably, an antibody selected from the group consisting of an anti-CD49a antibody, an anti-CD49b antibody, an anti-CD49c antibody, an anti-CD49d antibody, an anti-CD49e antibody, and an anti-CD49f antibody, or a fragment or a variant thereof may be used in the present invention.

As used as a target substance, DNA may be provided as naked DNA, preferably it is advantageous to provide such with a regulatory sequence such as a promoter in a vector (plasmid). In such a case, DNA is preferably operably linked to a controlling sequence.

The method according to the present invention preferably further comprises the step of providing a gene introduction reagent. The gene introduction reagent used herein is provided so as to be contacted to a cell. Use of gene introduction reagent is preferable, since it further enhances the introduction efficiency in the method according to the present invention. The provision of a gene introduction reagent is well known in the art, and for example, including the addition into an experimental system a solution with the gene introduction reagent dissolved therein, but is not limited thereto. Preferably, the gene introduction reagent is provided after the formation of a complex with DNA, a target substance. Preferably, gene introduction reagents are provided after the formation of a complex of a target substance and a DNA and cellular adhesion related agent has occurred. Although not wishing to be bound by any theory, it has been elucidated that such an order appears to enhance the efficiency of the introduction of a target substance into a cell on a solid support.

In an embodiment, a gene introduction reagent (for example, a cationic lipid)—a target substance complex includes a target substance (for example, DNA in an expression vector) and a gene introduction reagent, and may be solubilized in an appropriate solvent such as water or deionized water. This solution is spotted on a surface such as a slide, and thereby produces a surface with gene introduction reagent-target substance complex at a defined location. Thereafter, cellular adhesion related agent is appropriately added thereto. The spotted gene introduction reagent-target substance complex is attached onto a slide, and is sufficiently dried such that the spot remains attached to the location under the conditions used for the subsequent steps of the present method. For example, the gene introduction reagent-target substance complex is spotted, for example manually or using micro array producing apparatus, onto a slide such as a glass slide coated with poly-L-lysine (available from Sigma, Inc., or the like) or a chip. Thereafter, DNA spots may be attached to slides by maintaining the slides or chips at an elevated temperature (ie. greater than room temperature), or in vacuo and drying conditions. The length of time necessary for sufficient dryness to occur varies according to several factors such as the amount of the mixture located on the surface and temperature and humidity conditions used. In the present invention, the cellular adhesion related agent is preferably provided after the complex is attached.

The concentration of DNA present in the mixture may be determined in an experimental manner for the respective use, and generally in the range of about 0.01 μg/μl to about 0.2 μg/μl, in a specific embodiment, in the range of about 0.02 μg/μl to about 0.10 μg/μl. Alternatively, the concentration of DNA present in the gene introduction reagent-target substance complex may be in the range of about 0.01 μg/μl to about 0.5 μg/μl, in the range of about 0.01 μg/μl to about 0.4 μg/μl, in the range of about 0.01 μg/μl to about 0.3 μg/μl, and the like. Similarly, the concentration of another carrier macromolecule such as cellular adhesion related agents or gene introduction reagents may be experimentally determined for each application, and generally, is within the range of 0.01% to 0.5%, and in a particular embodiment, within the range of about 0.05% to 0.5%, about 0.05% to about 0.2%, or about 0.1% to about 0.2%. The final concentration of DNA in the cellular adhesion related agent-target substance complex (for example, DNA in the cellular adhesion related agent) is generally between about 0.02 μg/μl to about 0.1 μg/μl, and in another embodiment, the final concentration of DNA may be about 0.05 μg/μl.

When the DNA used is provided within a vector, the vector may be any type, such as a virus-based vector, and may contain the DNA of interest (DNA to be expressed in a cell), and thus may be expressed in the cell. Such a vector may be a CMV driven expression vector. Plasmid- or virus-based vectors such as pEGFP (Clontech) or pcDNA3 (Invitrogen), which are commercially available, may be used. In the present embodiment, after the spot comprising the gene introduction reagent-target substance complex is dried, the surface bearing the spot is covered with a lipid-based transfection reagent in an appropriate amount, and maintained (incubated) under the conditions appropriate for forming complex between DNA and the gene introduction reagent (for example, transfection reagent such as cationic lipid). Thereafter, the cellular adhesion related agent is provided, or alternatively, in a preferable embodiment, the cellular adhesion related agent is simultaneously provided therewith. In one embodiment, the resultant product is incubated for about twenty minutes at 25° C. Consequently, the gene introduction reagent is removed and the surface bearing DNA (DNA in a complex with the transfection reagent) is provided, and cells in an appropriate culture medium are plated on the surface. The resultant products (the surface bearing DNA and the plated cells) are maintained under conditions that cause the introduction of DNA into the cells plated thereon.

In the case of the present invention, about one to two cell cycles are sufficient for transfection, which varies depending on the type of a cell used and conditions applied, and the length of time for an appropriate particular combination may be readily determined by those skilled in the art in an experimental manner. After sufficient time is elapsed, with respect to the transfection efficiency, the expression of the produced encoded therein, and the effects on the cells, any methods known in the art may be used for evaluation thereof. For example, the detection of immunological fluorescence, or enzymatic immunological cytology, in situ hybridization, autoradiography, or any other means to detect the expression of mRNA or the encoded product, or the effects on the cell by the DNA introduced per se, and the like may be used for determining the above-mentioned parameters. When using immunological fluorescence to detect the expression of the protein encoded therein, an antibody which binds to the protein and is fluorescently labeled (which, for example, is added to the slide under an appropriate condition for binding of the antibody to a protein) and the location comprising the protein (spot or region on the surface) is detected by fluorescence to identify the same. The presence of fluorescence indicates that transfection occurred at the defined location presented by the fluorescence, and a protein encoded thereby is expressed. The presence of signals on a slide detected by the method used indicates that transfection and the expression of the encoded product or DNA introduction in the cell occurs at the particular location where the signal is detected. The identity of the DNA present on each particular location may be known or unknown; and thus when the expression occurs, the identity of the expressed protein may be known or unknown. Such information is preferably known, since it allows correlation to the conventional information.

All patents, published patent applications and publications cited herein are incorporated by reference as if set forth fully herein.

The preferred embodiments of the present invention have been heretofore described for a better understanding of the present invention. Hereinafter, the present invention will be described by way of examples. The examples described below are provided only for illustrative purposes. Accordingly, the scope of the present invention is not limited by the embodiments and examples specified herein except as by the appended claims.

### EXAMPLES

Hereinafter, the present invention will be described in greater detail by way of examples, though the present invention is not limited to the examples below. Reagents, supports, and the like were commercially available from Sigma (St. Louis, USA), Wako Pure Chemical Industries (Osaka, Japan), and Matsunami Glass (Kishiwada, Japan), unless otherwise specified.

### Example 1

### Preparation of Cellular Adhesion Related Agent Mixture

As candidates for a cellular adhesion related agent, various cellular adhesion related agents were prepared in the present Example as listed below. Antibodies were commercially available, or were prepared by raising antibodies against an integrin receptor. Polypeptides were commercially available, or were prepared by genetic manipulation and gene expression. For the CD29, and CD49 family, antibodies, may be prepared the using amino acid sequences set forth in SEQ ID NO: 3-14 by raising the same.

1) fibronectin (SEQ ID NO: 1);

2) CD49a antibody (IMMUNOTECH (a Coulter company), France, monoclonal antibody CD49a-VLA alfa1 Cat. No. 1599//COSMOBIO, Japan, mouse-ANTIratCD49a);

3) CD49b antibody (IMMUNOTECH (a Coulter company), France, monoclonal antibody CD49b Cat. No. 0717);

4) CD49c antibody (IMMUNOTECH (a Coulter company), France, monoclonal antibody CD49c Cat. No 2000);

5) CD49d antibody (IMMUNOTECH (a Coulter company), France, monoclonal antibody CD49d Cat. No 0764//ENDOGEN, USA, mouse-ANTIratCD49d);

6) CD49e antibody (IMMUNOTECH (a Coulter company), France, monoclonal antibody CD49e Cat. No. 0771);

7) CD49f antibody (IMMUNOTECH (a Coulter company), France, monoclonal antibody CD49f Cat. No. 0769//Antigenix America, USA, mouse-ANTIratCD49f, PRO. NO. MR496620); and

8) CD29 antibody (IMMUNOTECH (a Coulter company), France, monoclonal antibody CD29 Cat. No 1151).

Solutions of these antibodies, cellular adhesion related agent were prepared using the designated methods and volume and diluted to 100-5000 fold with PBS, and thereafter seeded onto the surface of a glass slide and dried. After drying, the slide was washed with PBS, and the complex of the gene introduction reagent and the gene printed thereon. The glass slides were plated with the cells of interest and solid phase gene introduction method was conducted. Anti-rat monoclonal antibody was used for PC12 cells.

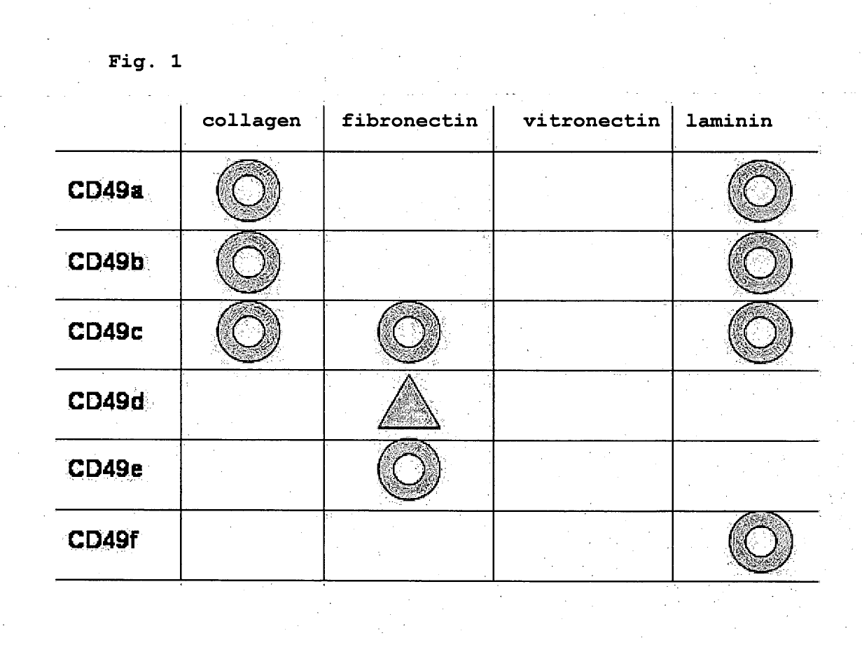

FIG. 1 shows the relationship between the integrin receptor and the extracellular matrix to be recognized thereby.

Plasmids were prepared as DNA for transfection. Plasmids, pEGFP-N1 and pDsRed2-N1 (both from BD Biosciences, Clontech, CA, USA) were used. In these plasmids, gene expression is under the control of cytomegalovirus (CMV) promoter. The plasmid DNA was amplified in E. coli (XL1 blue, Stratgene, TX, USA) and the amplified plasmid DNA was used as a complex partner. The DNA was dissolved in distilled water free from DNase and RNase.

The following transfection reagents were used: Effectene Transfection Reagent (cat. no. 301425, Qiagen, CA), TransFast™ Transfection Reagent (E2431, Promega, WI), Tfx™-20 Reagent (E2391, Promega, WI), SuperFect Transfection Reagent (301305, Qiagen, CA), PolyFect Transfection Reagent (301105, Qiagen, CA), LipofectAMINE 2000 Reagent (11668-019, Invitrogen corporation, CA), JetPEI (×4) conc. (101-30, Polyplus-transfection, France), and ExGen 500 (R0511, Fermentas Inc., MD). LipofectAMINE 2000 is preferably used, but is not limited thereto. These transfection reagents were added to the above-described DNA and actin acting substance in advance, or complexes thereof with the DNA were produced in advance.

The thus-obtained solution was used in an improvement test for transfection efficiency, as described below.

### Example 2

### Improvement in Transfection Efficiency in Liquid Phase

In the present Example, an improvement in the transfection efficiency in solid phase was observed. The protocol used in the present Example was followed according to the manufacturer's instructions for LipofectAMINE 2000.

In the present Example, the effects of these substances in liquid transfection using PC12 cells were investigated in the presence or absence of the cellular adhesion related agents as prepared above. PC12 (rat pheochromocytoma cells: ATCC CRL-1721) were cultured in DMEM/10% calf serum (GIBCO) supplemented with L-glutamine and penicillin/streptomycin.

Cellular adhesion related agents were stored as a stock of 200 μg/ml in PBS. All dilutions were made using PBS, ddH₂O, or Dulbecco's MEM medium. A series of dilutions, for example, 0.2 μg/mL, 0.067 μg/mL, 1.0 μg/mL, 5.0 μg/mL, 10.0 μg/mL, 50.0 μg/mL, and the like, were formulated.

As a result, these cellular adhesion related agents have been elucidated to have significant effects in enhancing the transfection efficiency in liquid transfection. In particular, collagen IV has been elucidated to have particularly significant effects upon transfection efficiency.

### Example 3

### Improvement in Transfection Efficiency in Solid Phase

(Protocol)

The final concentration of DNA was adjusted to 1 μg/μL. A cellular adhesion related agent was preserved as a stock having a concentration of 200 μg/mL in ddH₂O. All dilutions were made using PBS, ddH₂O, or Dulbecco's MEM. A series of dilutions, for example, 0.2 μg/mL, 0.067 μg/mL, 0.04 μg/mL, 1.0 μg/mL, and the like, were formulated.

Transfection reagents were used in accordance with instructions provided by each manufacturer.

Plasmid DNA was removed from a glycerol stock and amplified in 100 mL L-amp overnight. Qiaprep Miniprep or Qiagen Plasmid Purification Maxi was used to purify DNA in accordance with a standard protocol provided by the manufacturer.

In the present Example, the following cells were used to confirm an effect: HepG2 cells (RCB1648, RIKEN Cell Bank, JPN). These cells were cultured in DMEM/10% IFS containing L-glut and pen/strep.

(Dilution and DNA Spotting)

Transfection reagents and DNA were mixed to form a DNA-transfection reagent complex. The complex formation requires a certain period of time. Therefore, the mixture was spotted onto a solid phase support (e.g., a poly-L-lysine slide) using a pipette.

For complex formation and spot fixation, the slides were dried overnight in a vacuum dryer.

Cellular adhesion related agents may be used when forming the above-described complexes, but in the present Example, they were physically coated onto a solid support such as a poly-L-lysine slide. Solutions reconstituted with such a antibody-cellular adhesion related agents at a designated use and volume were diluted to 100 to 5,000 fold with PBS, and thereafter placed onto the surface of glass slides and dried. The plates were extensively washed with PBS, and a complex of a gene introduction and a gene was printed thereupon. The cells of interest were plated onto these glass slides to conducting solid phase gene introduction.

(Formulation of Mixed Solution and Application to Solid Phase Supports)

(Distribution of Cells)

Next, a protocol for adding cells will be described. Cells were distributed for transfection. The distribution was typically performed by low-pressure aspiration in a hood. A slide was placed on a dish, and a solution containing cells was added to the dish for transfection. The cells were distributed as follows.

The growing cells were adjusted to a concentration of 10⁷ cells/25 mL. The cells were plated on the slide in a 100×100×15 mm square Petri dish or a 100 mm (radius)×15 mm circular dish. Transfection was conducted for about 40 hours. This period of time corresponded to about 2 cell cycles. The slide was treated for immunofluorescence.

(Evaluation of Gene Introduction)

Gene introduction was evaluated by detection using, for example, immunofluorescence, fluorescence microscope examination, laser scanning, radioactive labels, and sensitive films, or emulsion.

When an expressed protein to be visualized is a fluorescent protein, such a protein can be observed with a fluorescence microscope and a photograph thereof can be taken. For large-sized expression arrays, slides may be scanned using a laser scanner for storage of data. If an expressed protein can be detected using fluorescent antibodies, an immunofluorescence protocol can be performed. If detection is based on radioactivity, the slide may be prepared as described above, and autoradiography using film or emulsion can be performed to detect radioactivity.

(Laser Scanning and Quantification of Fluorescence Intensity)

To quantify transfection efficiency, the present inventors used a DNA microarray scanner (GeneTAC UC4×4, Genomic Solutions Inc., MI). Total fluorescence intensity (arbitrary unit) was measured, and thereafter, fluorescence intensity per unit surface area was calculated.

(Results)

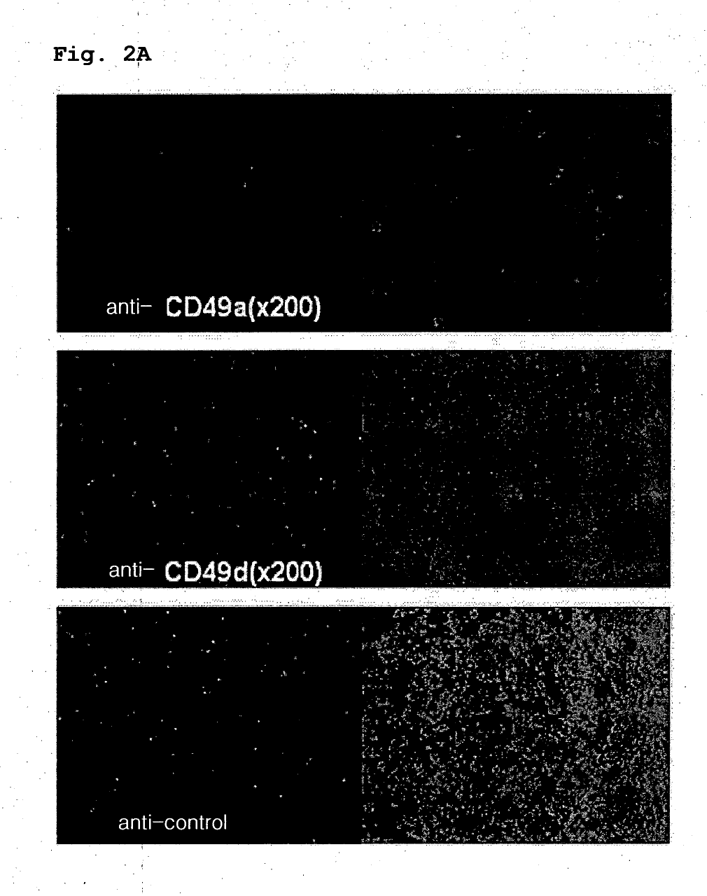

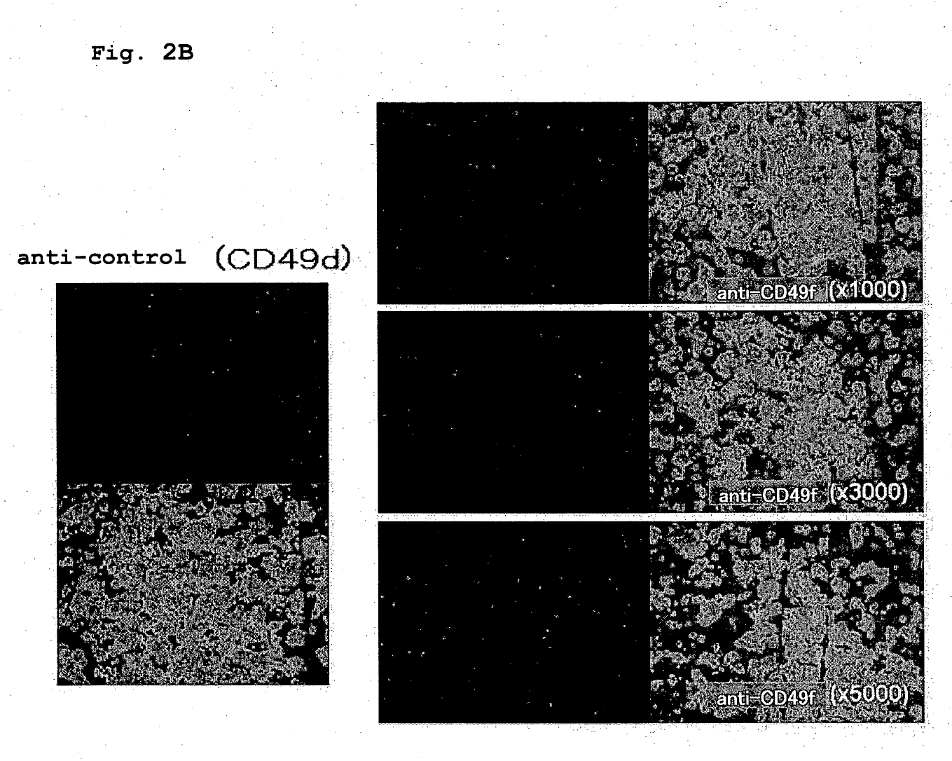

FIG. 2A depicts the results of experiments using a variety of cellular adhesion related agents and fibronectin when using HepG2 cells, as an example. FIG. 2B depicts another example of the results of transfection efficiency experiments using different cellular adhesion related agents (HLA, CD46, and CD54) in experiments using HepG2 cells.

As can be seen from the results, CD49 family antibodies caused significant transfection, as seen in the case of fibronectin. Accordingly, it was demonstrated that cellular adhesion related agents have significantly enhanced transfection efficiency.

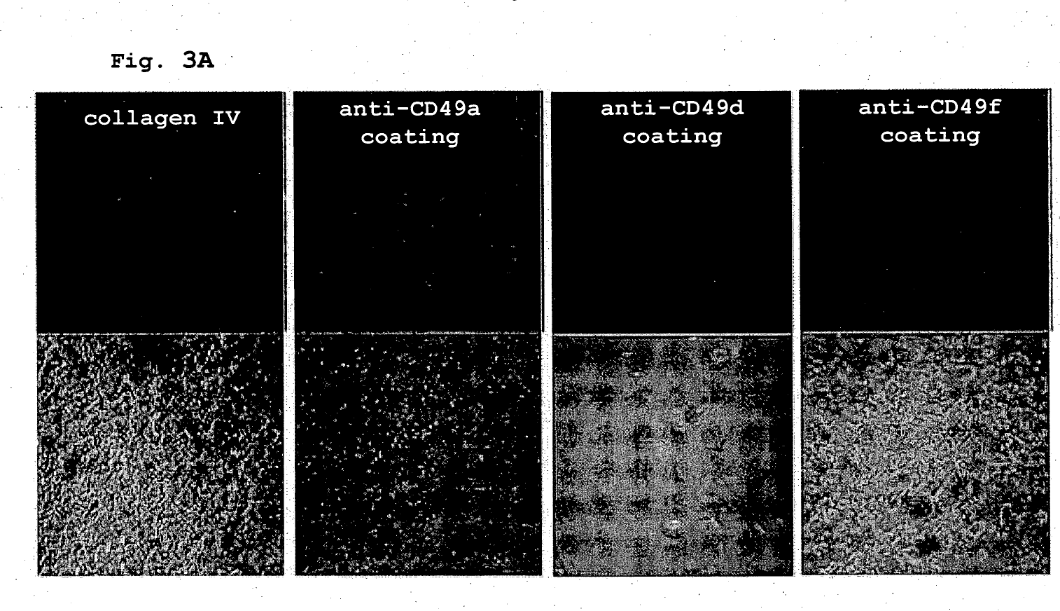

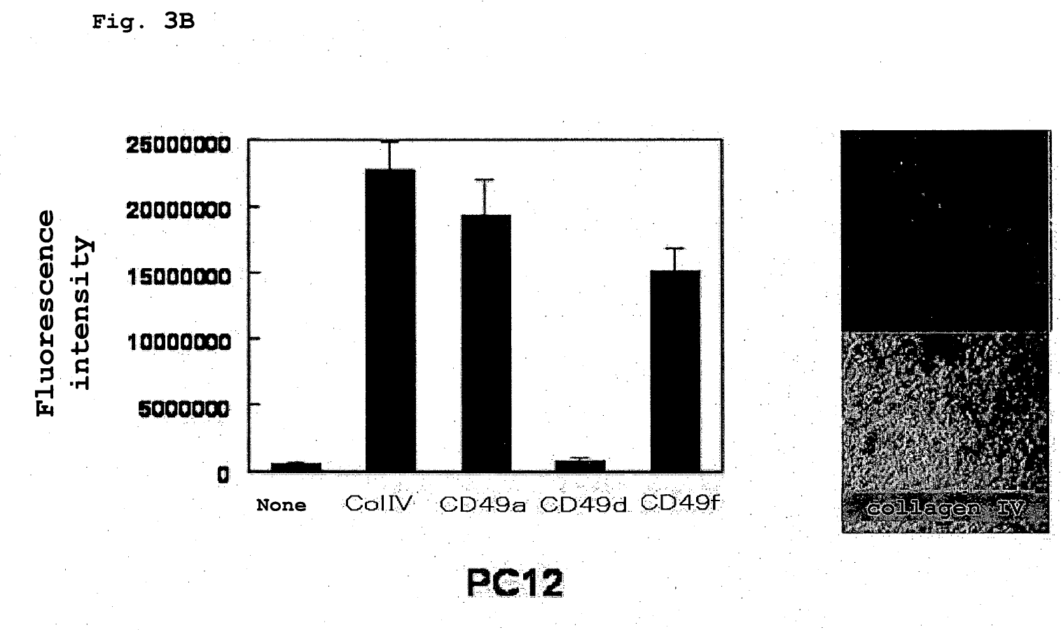

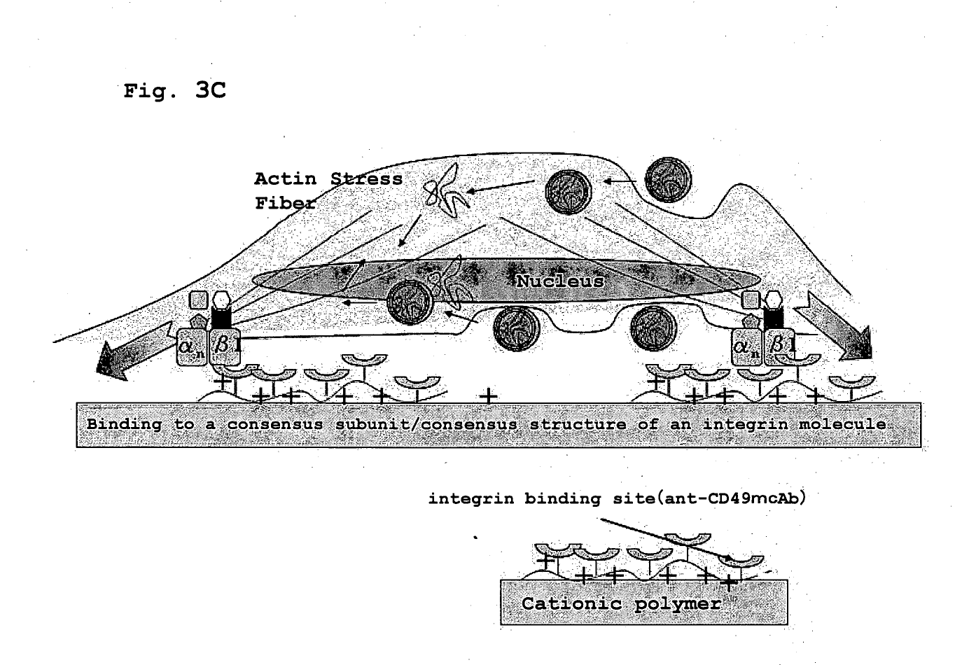

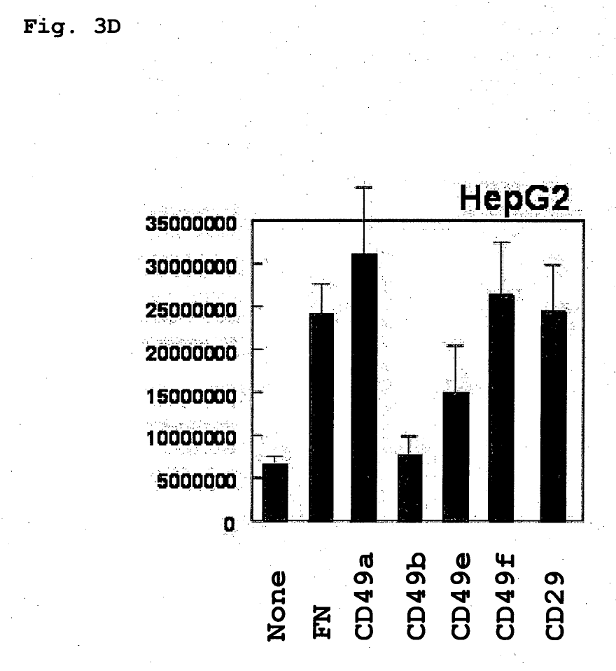

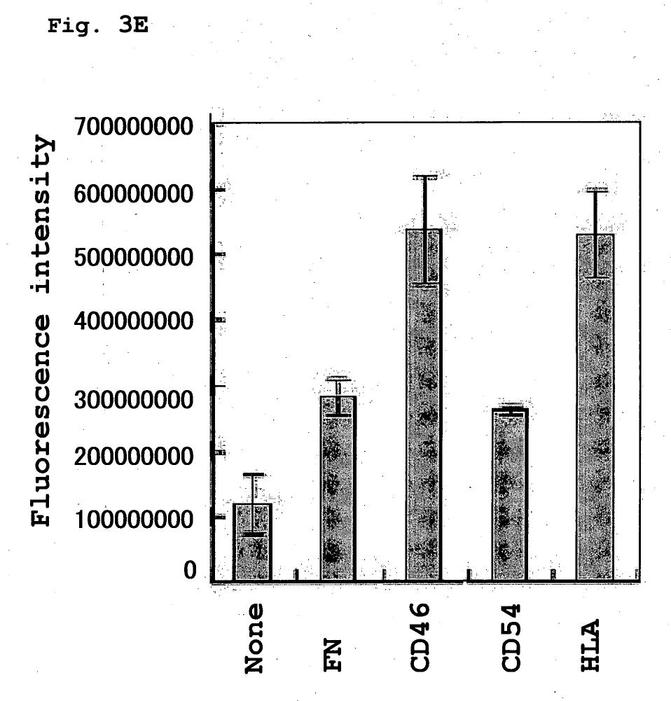

FIG. 3A shows the relationship between a variety of integrin receptors (CD49a, CD49d and CD49f) expressed on HepG2 cells with extracellular matrix proteins, and transfection efficiency on the surfaces coated with each extracellular matrix. FIGS. 3B-3E show the change in cells after transfection in the case of coating with a variety of cellular adhesion related agents.

The addition of cellular adhesion related agents enhances the efficiency of transfection. As such, in addition to HEK293 cells, HeLa cells, and 3T3 cells, which are conventionally reported to be transfectable, transfection efficiency comparable to that of HeLa cells and 3T3 cells was achieved in HepG2 cells, which have been believed to be difficult to transfect. Such results have never been achieved by means of conventional transfection systems, and thus the claimed invention has allowed enhancement of transfection efficiency in substantially all cells. As such, it can be said that systems that permit transfection of all cells, which can practically be used, has been provided for the first time. Furthermore, by means of employing solid conditions, cross-contamination has been significantly reduced. Accordingly, it has been demonstrated that the present invention is an appropriate method for producing integrated bioarrays when using a solid support.

Furthermore, when using a variety of plates, plates with coating were found to have reduced contamination compared with those without coating, and the transfection efficiency thereof was also enhanced.

Furthermore, the transfection efficiency has been elucidated to be enhanced with increased concentrations of antibody. It is found, however, that the efficiency reaches plateau at a predetermined concentration.

### Example 4

### Demonstration with PC12 Cells

PC12 cells, which are known as neuron-like cells, were used to observe whether or not differentiated cells attain effects according to the cellular adhesion related agents of the present invention.

According to Example 3, a variety of reagents and cells were prepared. PC12 cells, however, were prepared as follows:

PC12 (rat pheochromocytoma cells: ATCC CRL-1721) were cultured in a DMEM/10% calf serum (GIBCO) supplemented with L-glut and pen/strep. In order that the cell concentration be 10⁷ cells per 25 ml, proliferating cells were provided. Square Petri dishes of 100×100×5 mm or circular dishes of 100 mm radius×15 mm were used for plating the cells on a slide. Transfection was carried out for about forty-eight hours to treat slides for immunological fluorescence.

(Results)

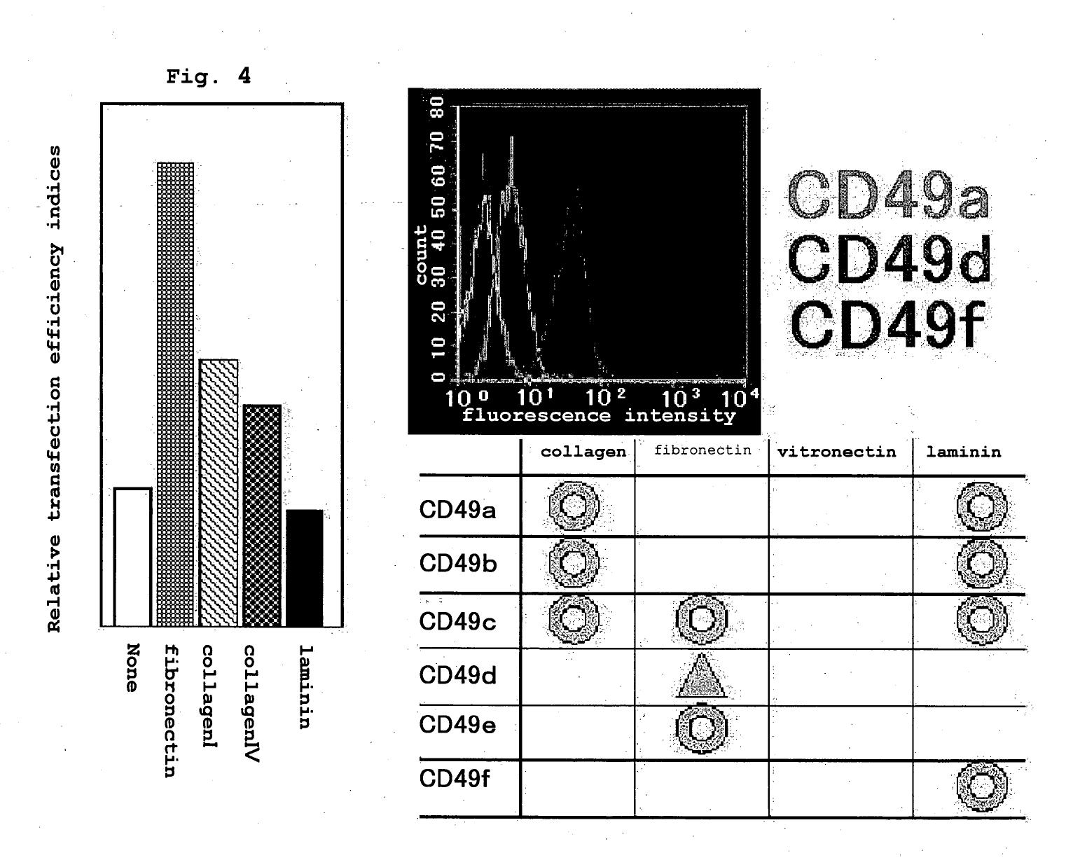

Results are shown in FIGS. 4-7. FIG. 4 depicts similar results to FIG. 3A in Example 3. As seen in the figure, it was elucidated that the cellular adhesion related agent according to the present invention enhances the transfection efficiency on PC12 cells, for which fibronectin has less effects.

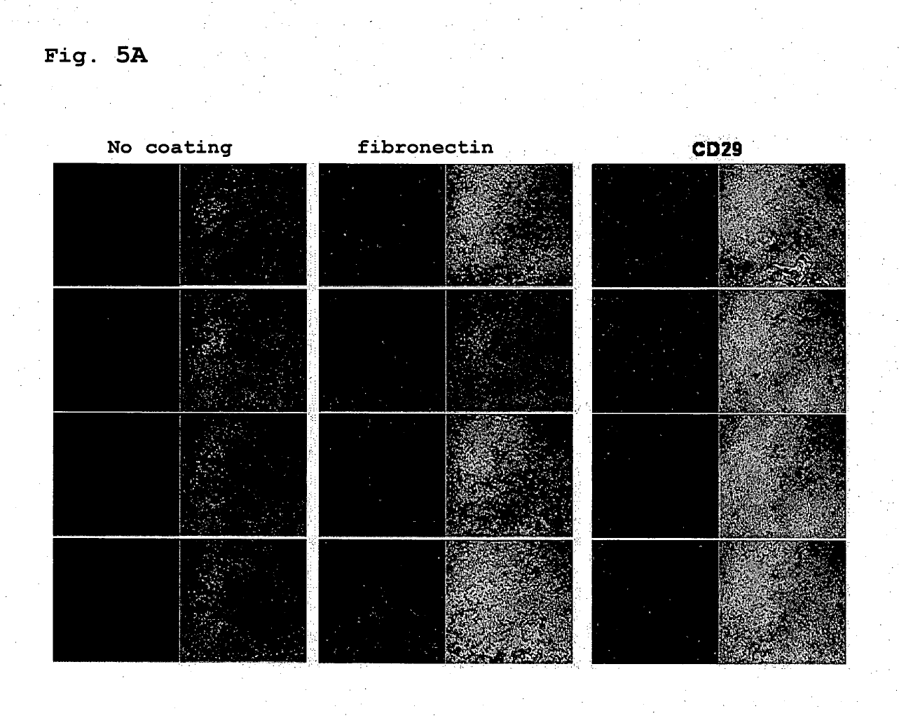

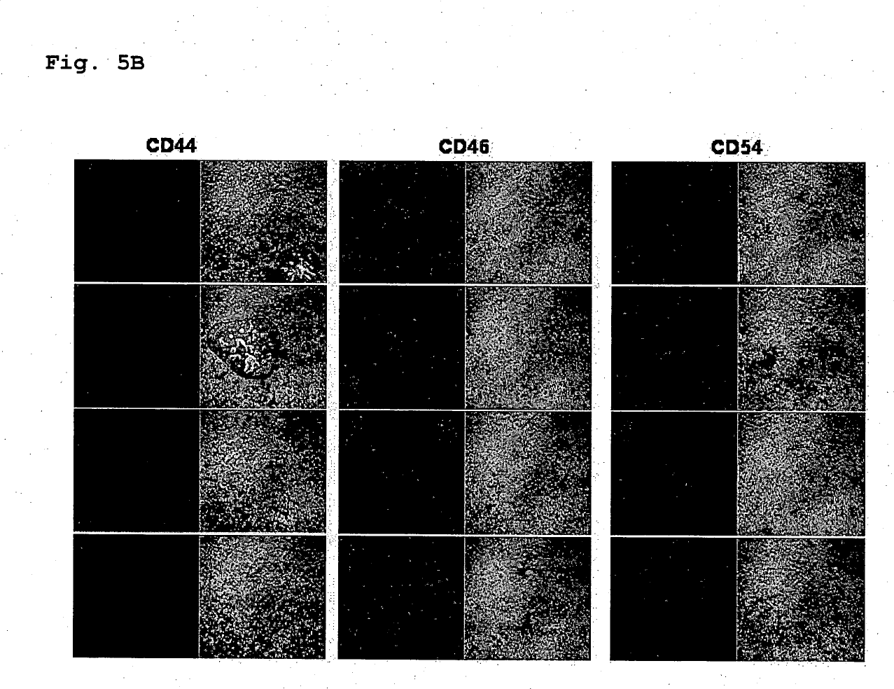

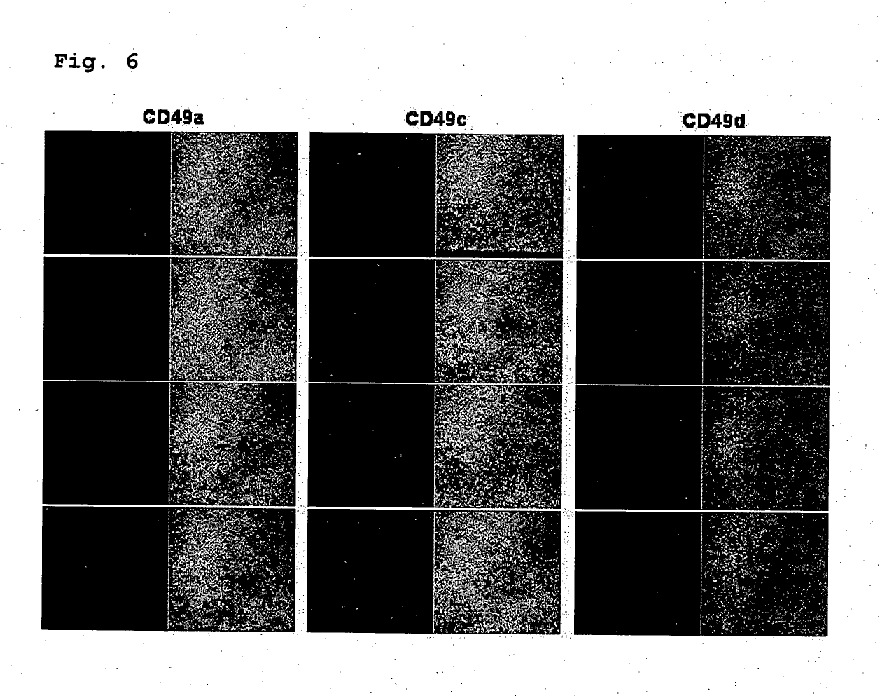

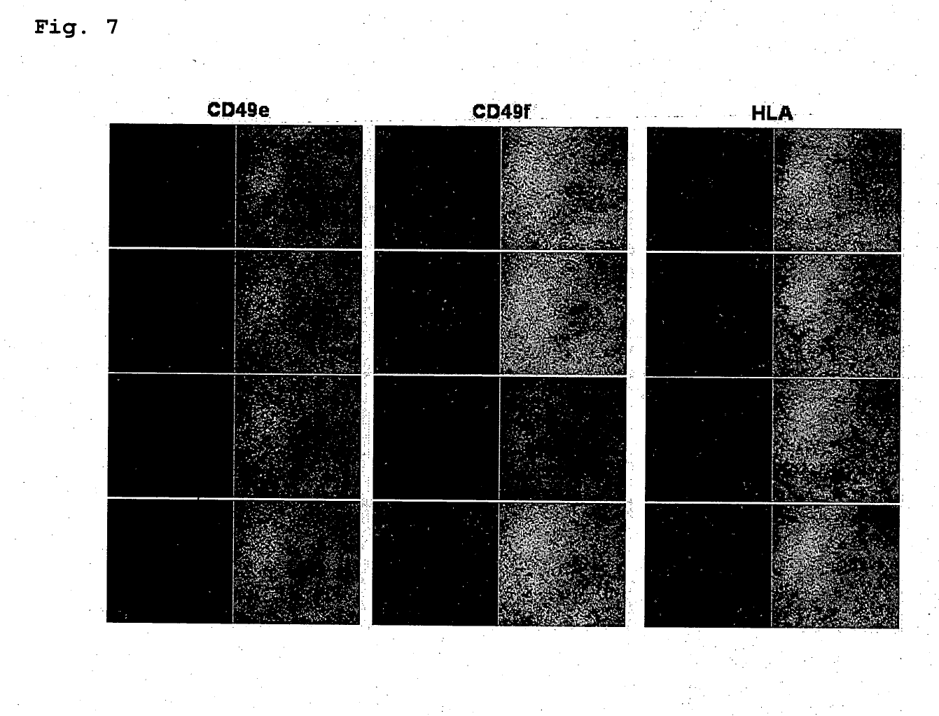

FIG. 5A-B depicts the state of cell adhesion. FIG. 6 shows a series of photographs comparing the states of transfection. FIG. 5A-B depicts cell adhesion of PC12 cells in Example 4. It shows adhesion inhibition and transfection of PC12 cells on a Type IV collagen coated surface using CD antibodies. Type IV collagen was coated onto poly-L-lysine coated slides, and thereafter, PC12 cells which had been previously contacted with an antibody solution were seeded and transfected with Lipofectamine 2000 according to the conventional protocol for use in liquid phase. FIG. 5A shows that anti-CD49a antibody significantly inhibited the adhesion of PC12 cells onto Type IV collagen coated surfaces. An anti-CD49d antibody shows the similar transfection efficiency as the control without antibody, by exhibiting no inhibition of adhesion of PC12 cells. As seen from FIG. 4, PC12 cells do not express CD49d. Furthermore, CD49d is a receptor against fibronectin, and thus it is believed that the adhesion of Type IV collagen coated surface is not inhibited. That is, the existence of an antibody per se does not affect the transfection efficiency. FIG. 5B similarly shows the difference in transfection efficiency in the presence of an anti-CD49f antibody. The values shown indicate the dilution of the antibody stock solution used for contacting the PC12 cells. As clearly seen from the figure, transfection efficiency is reduced in a manner depending on the concentration of an antibody to be contacted. CD49f is a receptor to laminin, and thus it is believed that adhesion to Type IV collagen coated surfaces is not inhibited. As seen from FIG. 4, PC12 cells express CD49f, and the transfection efficiency is enhanced by laminin, and thus anti-CD49f allows enhancement of transfection efficiency thereof (see FIGS. 6 and 7).

As seen from FIG. 5, transfection efficiency is reduced in a manner depending on the concentration of an antibody contacted thereto. CD49f is a receptor to laminin, and thus it is believed that adhesion to Type IV collagen coated surface is not inhibited. As seen from FIG. 4, PC12 cells express CD49f, and the transfection efficiency is enhanced by laminin, and thus anti-CD49f allows enhancement of transfection efficiency thereof (see FIGS. 6 and 7). Therefore, as shown in FIG. 7, it was demonstrated that anti-CD49f antibody was used to enhance transfection efficiency.

As such, the inhibition of cellular adhesion and the enhancement of transfection is directly related. Such relationship has not been known conventionally, and thus the present invention shows that by inhibiting the cellular adhesion, it was possible to enhance the transfection efficiency, and thus it is possible to enhance transfection efficiency in any cells by inhibiting cellular adhesion.

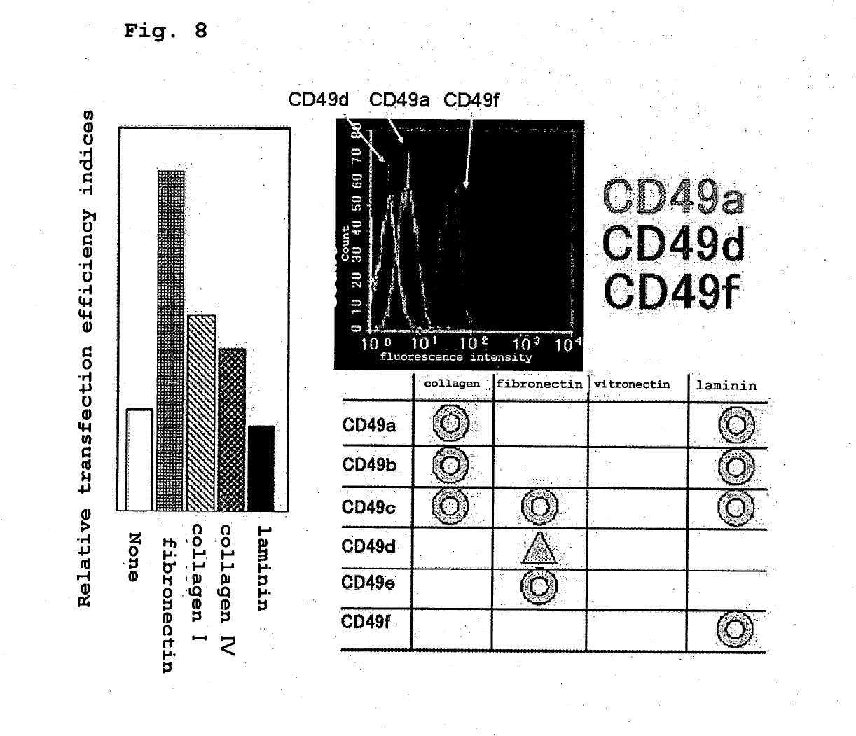

FIG. 7 shows the effects of coating of cellular adhesion related agent on a support. Coating with collagen type IV appears to enhance transfection efficiency. The present inventors developed a complex-salt system, which could be used to achieve solid phase transfection which makes it possible to obtain high transfection efficiency with various cell lines (HepG2) and special localization in high-density arrays. FIG. 8 depicts the outlines of solid transfection using a cellular adhesion related agent.

It was demonstrated that solid phase transfection can be used to achieve a “transfection patch” capable of being used for in vivo gene delivery and a solid phase transfection array (SPTA) for high-throughput genetic function research on HepG2 cells and PC12 cells.

Although a number of standard techniques are available for transfecting mammalian cells, it is known that it is inconvenient and difficult to introduce genetic material into cell lines such as HepG2 and PC12 and the like, as compared with cell lines such as HEK293, HeLa, and the like. Conventional viral vector delivery and electroporation techniques are each important. However, these techniques have the following inconveniences: potential toxicity (for the virus technique); difficulty in high-throughput analysis at the genomic scale; and limited applications in vivo studies (for electroporation).

The present inventors have developed a solid phase support fixed system which can be easily fixed to a solid phase support and has sustained-release capability and cell affinity, whereby most of the above-described drawbacks could be overcome.

The present inventors used their microprinting technique to fix a mixture of a selected genetic material, a transfection reagent, an appropriate cell adhesion molecule, and a salt onto a solid support. By culturing cells on a support having such a mixture fixed thereonto, the gene contained in the mixture was able to be taken in by the cultured cells. As a result, it became possible to allow support-adherent cells to take in DNA spatially separated therefrom.

As a result of this example, several important effects were achieved: high transfection efficiency (thereby making it possible to study a group of cells having a statistically significant scale); low cross contamination between regions having different DNA molecules (thereby making it possible to study the effects of different genes separately); the extended survival of transfected cells; high-throughput, compatibility and simple detection procedure. SPTA having these features serves as an appropriate basis for further studies.

To achieve the above-described objects, the present inventors studied different cell lines (HepG2 cells and PC12 cells), which are not efficiently transfected using fibronectin, as described above, with both our methodology including the present method (transfection in a solid phase system) and conventional liquid-phase transfection under a series of transfection conditions.

Transfection efficiency: transfection efficiency was determined as total fluorescence intensity per unit area. The results of liquid phase optimal to cell lines used were obtained. Next, these efficient transfection reagents were used to optimize a solid phase protocol. Several tendencies were observed. For cell lines which are readily transfectable (e.g., HEK293, HeLa, NIH3T3, etc.), the transfection efficiency observed in the solid phase protocol was slightly superior to, but essentially similar to, that of the standard liquid phase protocol.

However, for cells which are difficult to transfect (e.g., PC12 cells, HepG2 cells, etc.), we observed that transfection efficiency was increased up to 100 fold, while the features of the cells were retained under conditions optimized to the SPTA methodology. In the preliminary experimental results, HeLa cells show essentially high transfection, as described above.

The glass coating used is crucial for the achievement of high transfection efficiency on chips. It was found that PLL provided best results both for transfection efficiency and cross contamination. When a cellular adhesion related agent coating was not used, few transfectants were observed (all the other experimental conditions remained unchanged). Although not completely established, the cellular adhesion related agent probably plays a role in limiting the time which permits introduction of DNA.

Low cross contamination: apart from the higher transfection efficiency observed in the SPTA protocol, an important advantage of the present technique is to achieve an array of separated cells, in which selected genes are expressed in separate positions. The present inventors printed JetPEI and two different reporter genes mixed with fibronectin onto glass surface coated with fibronectin. The resultant transfection chip was subjected to appropriate cell culture. Expressed GFP and RFP were localized in regions, in which corresponding cDNA had been spotted, under the experimental conditions which had been found to be best. Substantially no cross contamination was observed.

This established technique is of particular importance in the context of cost-effective high-throughput gene function screening. Indeed, the small amounts of transfection reagent and DNA required, as well as the possible automatization of the entire process (from plasmid isolation to detection) increase the utility of the above presented method.

In conclusion, the present inventors successfully realized a PC12 cell and human mesenchymal stem cell (hMSC) transfection array in a system using complex-salt. With this technique, it will be possible to achieve high-throughput studies using the solid phase transfection, such as the elucidation of the genetic mechanism for differentiation of pluripotent stem cells. It was also elucidated that the detailed mechanism of the solid phase transfection as well as methodologies for the use of this technology for high throughput, real time gene expression monitoring can be applied for various purposes.

### Example 5

### Demonstration in a Human Mesenchymal Stem Cell

With respect to transfection, the solid transfection method as described in Example 3 was used to conduct the present Example.

The cells were prepared as follows: hMSCs (human mesenchymal stem cells, PT-2501, Cambrex BioScience Walkersville, Inc., MD) were used. In the case of human MSC cells, the cells were maintained in human mesenchymal cells basal medium (MSCGM BulletKit PT-3001, a commercially available medium from Cambrex BioScience Walkersville, Inc., MD).

As in the results of Example 4, the present inventors have realized a transfection array of human mesenchymal stem cells in a system using a cellular adhesion related agent. This allows high throughput study in a variety of research methods using solid transfection, such as clarification of the genetic mechanisms controlling the differentiation of pluripotent stem cells. It was elucidated that the detailed machinery of solid transfection, and methodology relating to the use of technology of high through put real time gene expression monitoring, are applicable to a variety of purposes.

### Example 6

### Demonstration in a Human Neuroblastoma Cell Line

SHSY5Y cells (human neuroblastoma: ATCC CRL-2266) were used to demonstrate the transfection efficiency of a neuroblastoma cell line.

Transfection was conducted according to Example 3. The cells were prepared by culture in a DMEM/10% FBS supplemented with L-glutamine and penicillin/streptomycin.

As in the results of Example 4, the present inventors have realized a transfection array of human mesenchymal stem cells in a system using a cellular adhesion related agent. This allows high throughput study in a variety of research methods using solid transfection, such as the clarification of the genetic mechanisms controlling the differentiation of pluripotent stem cells. It was elucidated that detailed machinery of solid transfection, and methodology relating to the use of technology of high through put real time gene expression monitoring, are applicable to a variety of purposes.

Although certain preferred embodiments have been described herein, it is not intended that such embodiments be construed as limitations of the scope of the invention except as set forth in the appended claims. Various other modifications and equivalents will be apparent to and can be readily made by those skilled in the art, after reading the description herein, without departing from the scope and spirit of this invention. All patents, published patent applications and publications cited herein are incorporated by reference as if set forth fully herein.

### INDUSTRIAL APPLICABILITY

According to the present invention, transfection efficiency can be increased either in a solid phase or in a liquid phase. The reagent for increasing transfection efficiency is useful for transfection in, particularly, solid phases. Accordingly, it is useful in any field using genetic engineering.

What is claimed is:

1. A composition for enhancing the introduction efficiency of a target substance into a cell, comprising a cellular adhesion related agent.

2. A composition for enhancing the introduction efficiency of a target substance into a cell according to claim 1 wherein the cellular adhesion related agent comprises an interaction substance interacting with a cellular adhesion molecule.

3. A composition according to claim 2, wherein the cellular adhesion molecule is an extracellular matrix.

4. A composition according to claim 2, wherein the cellular adhesion molecule is an integrin receptor.

5. A composition according to claim 2, wherein the cellular adhesion molecule comprises an RGD molecule.

6. A composition according to claim 2, wherein the interaction molecule raises an antigen-antibody reaction with a partner of the cellular adhesion molecule.

7. A composition according to claim 2, wherein the interaction molecule is an antibody or a derivative thereof.

8. A composition according to claim 2, wherein the interaction molecule is a monoclonal or polyclonal antibody.

9. A composition according to claim 2, wherein the interaction molecule comprises an antibody selected from the group consisting of an anti-CD49a antibody, an anti-CD49b antibody, an anti-CD49c antibody, an anti-CD49e antibody, and an anti-CD49f antibody.

10. A composition according to claim 1, wherein the target substance comprises a genetic material.

11. A composition according to claim 1, wherein the target substance comprises a nucleic acid molecule.

12. A composition according to claim 1, wherein the target substance comprises DNA.

13. A composition according to claim 4, wherein the integrin receptor is selected from the group consisting of CD49a, CD49b, CD49c, CD49d, CD49e, CD49f and CD29.

14. A composition according to claim 4, wherein the integrin receptor is selected from the group consisting of CD29, CD49a, CD49c, Cd49d, CD49e and CD49f.

15. A composition according to claim 4, wherein the integrin receptor interacts with a molecule selected from the group consisting of collagen, fibronectin, vitronectin and laminin.

16. A composition according to claim 1, wherein the cell comprises at least one cell selected from the group consisting of a stem cell and a differentiated cell.

17. A composition according to claim 1, wherein the cellular adhesion molecule is specifically expressed in the cell.

18. A composition according to claim 1, wherein the target substance is a genetic material and the composition further comprises a gene introduction reagent.

19. A composition according to claim 18, wherein the gene introduction reagent is selected from the group consisting of a cationic macromolecule, cationic lipid and calcium phosphate.

20. A composition according to claim 1, further comprising a particle.

21. A composition according to claim 20, wherein the particle comprises a gold colloid.

22. A composition according to claim 1 further comprising a salt.

23. A composition according to claim 22, wherein the salt is selected from the group consisting of salts comprised in a buffer and salts comprised in media.

24. A kit for enhancing gene introduction efficiency, comprising:
(a) a cellular adhesion related agent; and
(b) a gene introduction reagent.

25. A composition for introducing a target material to a cell, comprising:
(A) a target material; and
(B) a cellular adhesion related agent.

26. A composition according to claim 25, wherein the target material comprises a substance selected from the group consisting of DNA, RNA, polypeptide, sugar and a complex thereof.

27. A composition according to claim 25, wherein the target material comprises a DNA encoding a gene sequence to be transfected into the cell.

28. A composition according to claim 25 further comprising a gene introduction reagent.

29. A composition according to claim 25, wherein the cellular adhesion related agent comprises an interaction substance interacting with a cellular adhesion molecule.

30. A composition according to claim 25, wherein the cellular adhesion related agent comprises an antibody to a cellular adhesion molecule.

31. A composition according to claim 25 which is present as a liquid phase.

32. A composition according to claim 25 which is present as a solid phase.

33. A device for enhancing gene introduction efficiency of a target molecule into a cell, comprising:
(a) a target molecule; and
(b) a cellular adhesion related agent,
wherein the cellular adhesion related agent is immobilized onto a support.

34. A device according to claim 33, wherein the target substance comprises a substance selected from the group consisting of DNA, RNA, polypeptide, sugar and a complex thereof.

35. A device according to claim 33, wherein the target substance comprises a DNA encoding a gene sequence for the purpose of gene expression.

36. A device according to claim 33, further comprising a gene introduction reagent.

37. A device according to claim 36, wherein the cellular adhesion related agent comprises an interaction substance interacting with a cellular adhesion molecule.

38. A device according to claim 36, wherein the cellular adhesion related agent comprises an antibody against a cellular adhesion molecule.

39. A device according to claim 36, wherein the support is selected from the group consisting of a plate, a microwell plate, a tip, a slide glass, a film, a bead and metal.

40. A device according to claim 36, wherein the support is coated with a coating agent.

41. A device according to claim 40, wherein the coating agent comprises a substance selected from the group consisting of poly-L-lysine, silane, MAS, hydrophobic fluorine resin and metal.

42. A method for enhancing the introduction efficiency of a target substance into a cell, comprising the steps of:
A) providing a target substance;
B) providing a cellular adhesion related agent; and
C) contacting the target substance and the cellular adhesion related substance with the cell.

43. A method according to claim 42, wherein the target material comprises a substance selected from the group consisting of DNA, RNA, polypeptide, sugar and a complex thereof.

44. A method according to claim 43, wherein the target material comprises a DNA encoding a gene sequence to be transfected in the cell.

45. A method according to claim 42, further comprising a gene introduction reagent.

46. A method according to claim 42, wherein the cellular adhesion related agent comprises an interaction substance interacting with a cellular adhesion molecule.

47. A method according to claim 42, wherein the cellular adhesion related agent comprises an antibody to a cellular adhesion molecule.

48. A method according to claim 46, wherein the cellular adhesion molecule is an extracellular matrix molecule.

49. A method according to claim 42, wherein the method is conducted in a liquid phase.

50. A method according to claim 42, wherein the method is, conducted in a solid phase.

51. A method for enhancing the introduction efficiency of a target substance into a cell, comprising the steps of:
I) immobilizing a composition comprising
A) a target substance, and
B) a cellular adhesion molecule onto a support; and
II) contacting a cell to the composition on the support.

52. A method according to claim 51, further comprising the step of providing a gene introduction reagent, said gene introduction reagent being contacted with the cell.

53. A method according to claim 52, further comprising the step of forming a complex between the target substance and a gene introduction reagent after the provision thereof, wherein thereafter the cellular adhesion related agent is provided.

54. A method according to claim 51, wherein the cellular adhesion related agent comprises an interaction substance interacting with a cellular adhesion molecule.


SEQUENCE LISTING (14 sequences)

SEQ ID NO: 1 - DNA, 1929 bp, Homo sapiens
  CDS (1)..(1929): fibronectin 1
atgcttaggggtccggggcccgggctgctgctgctggccgtccagtgcctggggacagcggtgccctccacgggagcctcgaagagcaagaggcaggctcagcaaatggttcagccccagtccccggtggctgtcagtcaaagcaagcccggttgttatgacaatggaaaacactatcagataaatcaacagtgggagcggacctacctaggcaatgcgttggtttgtacttgttatggaggaagccgaggttttaactgcgagagtaaacctgaagctgaagagacttgctttgacaagtacactgggaacacttaccgagtgggtgacacttatgagcgtcctaaagactccatgatctgggactgtacctgcatcggggctgggcgagggagaataagctgtaccatcgcaaaccgctgccatgaagggggtcagtcctacaagattggtgacacctggaggagaccacatgagactggtggttacatgttagagtgtgtgtgtcttggtaatggaaaaggagaatggacctgcaagcccatagctgagaagtgttttgatcatgctgctgggacttcctatgtggtcggagaaacgtgggagaagccctaccaaggctggatgatggtagattgtacttgcctgggagaaggcagcggacgcatcacttgcacttctagaaatagatgcaacgatcaggacacaaggacatcctatagaattggagacacctggagcaagaaggataatcgaggaaacctgctccagtgcatctgcacaggcaacggccgaggagagtggaagtgtgagaggcacacctctgtgcagaccacatcgagcggatctggccccttcaccgatgttcgtgcagctgtttaccaaccgcagcctcacccccagcctcctccctatggccactgtgtcacagacagtggtgtggtctactctgtggggatgcagtggctgaagacacaaggaaataagcaaatgctttgcacgtgcctgggcaacggagtcagctgccaagagacagctgtaacccagacttacggtggcaactcaaatggagagccatgtgtcttaccattcacctacaatggcaggacggacagcacaacttcgaattatgagcaggaccagaaatactctttctgcacagaccacactgttttggttcagactcgaggaggaaattccaatggtgccttgtgccacttccccttcctatacaacaaccacaattacactgattgcacttctgagggcagaagagacaacatgaagtggtgtgggaccacacagaactatgatgccgaccagaagtttgggttctgccccatggctgcccacgaggaaatctgcacaaccaatgaaggggtcatgtaccgcattggagatcagtgggataagcagcatgacatgggtcacatgatgaggtgcacgtgtgttgggaatggtcgtggggaatggacatgcattgcctactcgcagcttcgagatcagtgcattgttgatgacatcacttacaatgtgaacgacacattccacaagcgtcatgaagaggggcacatgctgaactgtacatgcttcggtcagggtcggggcaggtggaagtgtgatcccgtcgaccaatgccaggattcagagactgggacgttttatcaaattggagattcatgggagaagtatgtgcatggtgtcagataccagtgctactgctatggccgtggcattggggagtggcattgccaacctttacagacctatccaagctcaagtggtcctgtcgaagtatttatcactgagactccgagtcagcccaactcccaccccatccagtggaatgcaccacagccatctcacatttccaagtacattctcaggtggagacctgtgagtatcccacccagaaaccttggatactga

SEQ ID NO: 2 - protein, 642 aa, Homo sapiens
MLRGPGPGLLLLAVQCLGTAVPSTGASKSKRQAQQMVQPQSPVAVSQSKPGCYDNGKHYQINQQWERTYLGNALVCTCYGGSRGFNCESKPEAEETCFDKYTGNTYRVGDTYERPKDSMIWDCTCIGAGRGRISCTIANRCHEGGQSYKIGDTWRRPHETGGYMLECVCLGNGKGEWTCKPIAEKCFDHAAGTSYVVGETWEKPYQGWMMVDCTCLGEGSGRITCTSRNRCNDQDTRTSYRIGDTWSKKDNRGNLLQCICTGNGRGEWKCERHTSVQTTSSGSGPFTDVRAAVYQPQPHPQPPPYGHCVTDSGVVYSVGMQWLKTQGNKQMLCTCLGNGVSCQETAVTQTYGGNSNGEPCVLPFTYNGRTDSTTSNYEQDQKYSFCTDHTVLVQTRGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSSSGPVEVFITETPSQPNSHPIQWNAPQPSHISKYILRWRPVSIPPRNLGY

SEQ ID NO: 3 - protein, 798 aa, Homo sapiens
MNLQPIFWIGLISSVCCVFAQTDENRCLKANAKSCGECIQAGPNCGWCTNSTFLQEGMPTSARCDDLEALKKKGCPPDDIENPRGSKDIKKNKNVTNRSKGTAEKLKPEDITQIQPQQLVLRLRSGEPQTFTLKFKRAEDYPIDLYYLMDLSYSMKDDLENVKSLGTDLMNEMRRITSDFRIGFGSFVEKTVMPYISTTPAKLRNPCTSEQNCTSPFSYKNVLSLTNKGEVFNELVGKQRISGNLDSPEGGFDAIMQVAVCGSLIGWRNVTRLLVFSTDAGFHFAGDGKLGGIVLPNDGQCHLENNMYTMSHYYDYPSIAHLVQKLSENNIQTIFAVTEEFQPVYKELKNLIPKSAVGTLSANSSNVIQLIIDAYNSLSSEVILENGKLSEGVTISYKSYCKNGVNGTGENGRKCSNISIGDEVQFEISITSNKCPKKDSDSFKIRPLGFTEEVEVILQYICECECQSEGIPESPKCHEGNGTFECGACRCNEGRVGRHCECSTDEVNSEDMDAYCRKENSSEICSNNGECVCGQCVCRKRDNTNEIYSGKFCECDNFNCDRSNGLICGGNGVCKCRVCECNPNYTGSACDCSLDTSTCEASNGQICNGRGICECGVCKCTDPKFQGQTCEMCQTCLGVCAEHKECVQCRAFNKGEKKDTCTQECSYFNITKVESRDKLPQPVQPDPVSHCKEKDVDDCWFYFTYSVNGNNEVMVHVVENPECPTGPDIIPIVAGVVAGIVLIGLALLLIWKLLMIIHDRREFAKFEKEKMNAKWDTGENPIYKSAVTTVVNPKYEGK

SEQ ID NO: 4 - protein, 1151 aa, Homo sapiens
FNVDVKNSMTFSGPVEDMFGYTVQQYENEEGKWVLIGSPLVGQPKNRTGDVYKCPVGRGESLPCVKLDLPVNTSIPNVTEVKENMTFGSTLVTNPNGGFLACGPLYAYRCGHLHYTTGICSDVSPTFQVVNSIAPVQECSTQLDIVIVLDGSNSIYPWDSVTAFLNDLLKRMDIGPKQTQVGIVQYGENVTHEFNLNKYSSTEEVLVAAKKIVQRGGRQTMTALGTDTARKEAFTEARGARRGVKKVMVIVTDGESHDNHRLKKVIQDCEDENIQRFSIAILGSYNRGNLSTEKFVEEIKSIASEPTEKHFFNVSDELALVTIVKTLGERIFALEATADQSAASFEMEMSQTGFSAHYSQDWVMLGAVGAYDWNGTVVMQKASQIIIPRNTTFNVESTKKNEPLASYLGYTVNSATASSGDVLYIAGQPRYNHTGQVIIYRMEDGNIKILQTLSGEQIGSYFGSILTTTDIDKDSNTDILLVGAPMYMGTEKEEQGKVYVYALNQTRFEYQMSLEPIKQTCCSSRQHNSCTTENKNEPCGARFGTAIAAVKDLNLDGFNDIVIGAPLEDDHGGAVYIYHGSGKTIRKEYAQRIPSGGDGKTLKFFGQSIHGEMDLNGDGLTDVTIGGLGGAALFWSRDVAVVKVTMNFEPNKVNIQKKNCHMEGKETVCINATVCFEVKLKSKEDTIYEADLQYRVTLDSLRQISRSFFSGTQERKVQRNITVRKSECTKHSFYMLDKHDFQDSVRITLDFNLTDPENGPVLDDSLPNSVHEYIPFAKDCGNKEKCISDLSLHVATTEKDLLIVRSQNDKFNVSLTVKNTKDSAYNTRTIVHYSPNLVFSGIEAIQKDSCESNHNITCKVGYPFLRRGEMVTFKILFQFNTSYLMENVTIYLSATSDSEEPPETLSDNVVNISIPVKYEVGLQFYSSASEYHISIAANETVPEVINSTEDIGNEINIFYLIRKSGSFPMPELKLSISFPNMTSNGYPVLYPTGLSSSENANCRPHIFEDPFSINSGKKMTTSTDHLKRGTILDCNTCKFATITCNLTSSDISQVNVSLILWKPTFIKSYFSSLNLTIRGELRSENASLVLSSSNQKRELAIQISKDGLPGRVPLWVILLSAFAGLLLLMLLILALWKIGFFKRPLKKKMEK

SEQ ID NO: 5 - protein, 1181 aa, Homo sapiens
MGPERTGAAPLPLLLVLALSQGILNCCLAYNVGLPEAKIFSGPSSEQFGYAVQQFINPKGNWLLVGSPWSGFPENRMGDVYKCPVDLSTATCEKLNLQTSTSIPNVTEMKTNMSLGLILTRNMGTGGFLTCGPLWAQQCGNQYYTTGVCSDISPDFQLSASFSPATQPCPSLIDVVVVCDESNSIYPWDAVKNFLEKFVQGLDIGPTKTQVGLIQYANNPRVVFNLNTYKTKEEMIVATSQTSQYGGDLTNTFGAIQYARKYAYSAASGGRRSATKVMVVVTDGESHDGSMLKAVIDQCNHDNILRFGIAVLGYLNRNALDTKNLIKEIKAIASIPTERYFFNVSDEAALLEKAGTLGEQIFSIEGTVQGGDNFQMEMSQVGFSADYSSQNDILMLGAVGAFGWSGTIVQKTSHGHLIFPKQAFDQILQDRNHSSYLGYSVAAISTGESTHFVAGAPRANYTGQIVLYSVNENGNITVIQAHRGDQIGSYFGSVLCSVDVDKDTITDVLLVGAPMYMSDLKKEEGRVYLFTIKEGILGQHQFLEGPEGIENTRFGSAIAALSDINMDGFNDVIVGSPLENQNSGAVYIYNGHQGTIRTKYSQKILGSDGAFRSHLQYFGRSLDGYGDLNGDSITDVSIGAFGQVVQLWSQSIADVAIEASFTPEKITLVNKNAQIILKLCFSAKFRPTKQNNQVAIVYNITLDADGFSSRVTSRGLFKENNERCLQKNMVVNQAQSCPEHIIYIQEPSDVVNSLDLRVDISLENPGTSPALEAYSETAKVFSIPFHKDCGEDGLCISDLVLDVRQIPAAQEQPFIVSNQNKRLTFSVTLKNKRESAYNTGIVVDFSENLFFASFSLPVDGTEVTCQVAASQKSVACDVGYPALKREQQVTFTINFDFNLQNLQNQASLSFQALSESQEENKADNLVNLKIPLLYDAEIHLTRSTNINFYEISSDGNVPSIVHSFEDVGPKFIFSLKVTTGSVPVSMATVIIHIPQYTKEKNPLMYLTGVQTDKAGDISCNADINPLKIGQTSSSVSFKSENFRHTKELNCRTASCSNVTCWLKDVHMKGEYFVNVTTRIWNGTFASSTFQTVQLTAAAEINTYNPEIYVIEDNTVTIPLMIMKPDEKAEVPTGVIIGSIIAGILLLLALVAILWKLGFFKRKYEKMTKNPDEIDETTELSS

SEQ ID NO: 6 - protein, 1051 aa, Homo sapiens
MGPGPSRAPRAPRLMLCALALMVAAGGCVVSAFNLDTRFLVVKEAGNPGSLFGYSVALHRQTERQQRYLLLAGAPRELAVPDGYTNRTGAVYLCPLTAHKDDCERMNITVKNDPGHHIIEDMWLGVTVASQGPAGRVLVCAHRYTQVLWSGSEDQRRMVGKCYVRGNDLELDSSDDWQTYHNEMCNSNTDYLETGMCQLGTSGGFTQNTVYFGAPGAYNWKGNSYMIQRKEWDLSEYSYKDPEDQGNLYIGYTMQVGSFILHPKNITIVTGAPRHRHMGAVFLLSQEAGGDLRRRQVLEGSQVGAYFGSAIALADLNNDGWQDLLVGAPYYFERKEEVGGAIYVFMNQAGTSFPAHPSLLLHGPSGSAFGLSVASIGDINQDGFQDIAVGAPFEGLGKVYIYHSSSKGLLRQPQQVIHGEKLGLPGLATFGYSLSGQMDVDENFYPDLLVGSLSDHIVLLRARPVINIVHKTLVPRPAVLDPALCTATSCVQVELCFAYNQSAGNPNYRRNITLAYTLEADRDRRPPRLRFAGSESAVFHGFFSMPEMRCQKLELLLMDNLRDKLRPIIISMNYSLPLRMPDRPRLGLRSLDAYPILNQAQALENHTEVQFQKECGPDNKCESNLQMRAAFVSEQQQKLSRLQYSRDVRKLLLSINVTNTRTSERSGEDAHEALLTLVVPPALLLSSVRPPGACQANETIFCELGNPFKRNQRMELLIAFEVIGVTLHTRDLQVQLQLSTSSHQDNLWPMILTLLVDYTLQTSLSMVNHRLQSFFGGTVMGESGMKTVEDVGSPLKYEFQVGPMGEGLVGLGTLVLGLEWPYEVSNGKWLLYPTEITVHGNGSWPCRPPGDLINPLNLTLSDPGDRPSSPQRRRRQLDPGGGQGPPPVTLAAAKKAKSETVLTCATGRAHCVWLECPIPDAPVVTNVTVKARVWNSTFIEDYRDFDRVRVNGWATLFLRTSIPTINMENKTTWFSVDIDSELVEELPAEIELWLVLVAVGAGLLLLGLIILLLWKCGFFKRARTRALYEAKRQKAEMKSQPSETERLTDDY

SEQ ID NO: 7 - protein, 1038 aa, Homo sapiens
MFPTESAWLGKRGANPGPEAAVRETVMLLLCLGVPTGRPYNVDTESALLYQGPHNTLFGYSVVLHSHGANRWLLVGAPTANWLANASVINPGAIYRCRIGKNPGQTCEQLQLGSPNGEPCGKTCLEERDNQWLGVTLSRQPGENGSIVTCGHRWKNIFYIKNENKLPTGGCYGVPPDLRTELSKRIAPCYQDYVKKFGENFASCQAGISSFYTKDLIVMGAPGSSYWTGSLFVYNITTNKYKAFLDKQNQVKFGSYLGYSVGAGHFRSQHTTEVVGGAPQHEQIGKAYIFSIDEKELNILHEMKGKKLGSYFGASVCAVDLNADGFSDLLVGAPMQSTIREEGRVFVYINSGSGAVMNAMETNLVGSDKYAARFGESIVNLGDIDNDGFEDVAIGAPQEDDLQGAIYIYNGRADGISSTFSQRIEGLQISKSLSMFGQSISGQIDADNNGYVDVAVGAFRSDSAVLLRTRPVVIVDASLSHPESVNRTKFDCVENGWPSVCIDLTLCFSYKGKEVPGYIVLFYNMSLDVNRKAESPPRFYFSSNGTSDVITGSIQVSSREANCRTHQAFMRKDVRDILTPIQIEAAYHLGPHVISKRSTEEFPPLQPILQQKKEKDIMKKTINFARFCAHENCSADLQVSAKIGFLKPHENKTYLAVGSMKTLMLNVSLFNAGDDAYETTLHVKLPVGLYFIKILELEEKQINCEVTDNSGVVQLDCSIGYIYVDHLSRIDISFLLDVSSLSRAEEDLSITVHATCENEEEMDNLKHSRVTVAIPLKYEVKLTVHGFVNPTSFVYGSNDENEPETCMVEKMNLTFHVINTGNSMAPNVSVEIMVPNSFSPQTDKLFNILDVQTTTGECHFENYQRVCALEQQKSAMQTLKGIVRFLSKTDKRLLYCIKADPHCLNFLCNFGKMESGKEASVHIQLEGRPSILEMDETSALKFEIRATGFPEPNPRVIELNKDENVAHVLLEGLHHQRPKRYFTIVIISSSLLLGLIVLLLISYVMWKAGFFKRQYKSILQEENRRDSWSYINSKSNDD

SEQ ID NO: 8 - protein, 1049 aa, Homo sapiens
MGSRTPESPLHAVQLRWGPRRRPPLLPLLLLLLPPPPRVGGFNLDAEAPAVLSGPPGSFFGFSVEFYRPGTDGVSVLVGAPKANTSQPGVLQGGAVYLCPWGASPTQCTPIEFDSKGSRLLESSLSSSEGEEPVEYKSLQWFGATVRAHGSSILACAPLYSWRTEKEPLSDPVGTCYLSTDNFTRILEYAPCRSDFSWAAGQGYCQGGFSAEFTKTGRVVLGGPGSYFWQGQILSATQEQIAESYYPEYLINLVQGQLQTRQASSIYDDSYLGYSVAVGEFSGDDTEDFVAGVPKGNLTYGYVTILNGSDIRSLYNFSGEQMASYFGYAVAATDVNGDGLDDLLVGAPLLMDRTPDGRPQEVGRVYVYLQHPAGIEPTPTLTLTGHDEFGRFGSSLTPLGDLDQDGYNDVAIGAPFGGETQQGVVFVFPGGPGGLGSKPSQVLQPLWAASHTPDFFGSALRGGRDLDGNGYPDLIVGSFGVDKAVVYRGRPIVSASASLTIFPAMFNPEERSCSLEGNPVACINLSFCLNASGKHVADSIGFTVELQLDWQKQKGGVRRALFLASRQATLTQTLLIQNGAREDCREMKIYLRNESEFRDKLSPIHIALNFSLDPQAPVDSHGLRPALHYQSKSRIEDKAQILLDCGEDNICVPDLQLEVFGEQNHVYLGDKNALNLTFHAQNVGEGGAYEAELRVTAPPEAEYSGLVRHPGNFSSLSCDYFAVNQSRLLVCDLGNPMKAGASLWGGLRFTVPHLRDTKKTIQFDFQILSKNLNNSQSDVVSFRLSVEAQAQVTLNGVSKPEAVLFPVSDWHPRDQPQKEEDLGPAVHHVYELINQGPSSISQGVLELSCPQALEGQQLLYVTRVTGLNCTTNHPINPKGLELDPEGSLHHQQKREAPSRSSASSGPQILKCPEAECFRLRCELGPLHQQESQSLQLHFRVWAKTFLQREHQPFSLQCEAVYKALKMPYRILPRQLPQKERQVATAVQWTKAEGSYGVPLWIIILAILFGLLLLGLLIYILYKLGFFKRSLPYGTAMEKAQLKPPATSDA

SEQ ID NO: 9 - protein, 1073 aa, Homo sapiens
MAAAGQLCLLYLSAGLLSRLGAAFNLDTREDNVIRKYGDPGSLFGFSLAMHWQLQPEDKRLLLVGAPRGEALPLQRANRTGGLYSCDITARGPCTRIEFDNDADPTSESKEDQWMGVTVQSQGPGGKVVTCAHRYEKRQHVNTKQESRDIFGRCYVLSQNLRIEDDMDGGDWSFCDGRLRGHEKFGSCQQGVAATFTKDFHYIVFGAPGTYNWKGIVRVEQKNNTFFDMNIFEDGPYEVGGETEHDESLVPVPANSYLGFSLDSGKGIVSKDEITFVSGAPRANHSGAVVLLKRDMKSAHLLPEHIFDGEGLASSFGYDVAVVDLNKDGWQDIVIGAPQYFDRDGEVGGAVYVYMNQQGRWNNVKPIRLNGTKDSMFGIAVKNIGDINQDGYPDIAVGAPYDDLGKVFIYHGSANGINTKPTQVLKGISPYFGYSIAGNMDLDRNSYPDVAVGSLSDSVTIFRSRPVINIQKTITVTPNRIDLRQKTACGAPSGICLQVKSCFEYTANPAGYNPSISIVGTLEAEKERRKSGLSSRVQFRNQGSEPKYTQELTLKRQKQKVCMEETLWLQDNIRDKLRPIPITASVEIQEPSSRRRVNSLPEVLPILNSDEPKTAHIDVHFLKEGCGDDNVCNSNLKLEYKFCTREGNQDKFSYLPIQKGVPELVLKDQKDIALEITVTNSPSNPRNPTKDGDDAHEAKLIATFPDTLTYSAYRELRAFPEKQLSCVANQNGSQADCELGNPFKRNSNVTFYLVLSTTEVTFDTPYLDINLKLETTSNQDNLAPITAKAKVVIELLLSVSGVAKPSQVYFGGTVVGEQAMKSEDEVGSLIEYEFRVINLGKPLTNLGTATLNIQWPKEISNGKWLLYLVKVESKGLEKVTCEPQKEINSLNLTESHNSRKKREITEKQIDDNRKFSLFAERKYQTLNCSVNVNCVNIRCPLRGLDSKASLILRSRLWNSTFLEEYSKLNYLDILMRAFIDVTAAAENIRLPNAGTQVRVTVFPSKTVAQYSGVPWWIILVAILAGILMLALLVFILWKCGFFKRNKKDHYDATYHKAEIHAQPSDKERLTSDA

SEQ ID NO: 10 - protein, 789 aa, Homo sapiens
MNLQPIFWIGLISSVCCVFAQTDENRCLKANAKSCGECIQAGPNCGWCTNSTFLQEGMPTSARCDDLEALKKKGCPPDDIENPRGSKDIKKNKNVTNRSKGTAEKLKPEDITQIQPQQLVLRLRSGEPQTFTLKFKRAEDYPIDLYYLMDLSYSMKDDLENVKSLGTDLMNEMRRITSDFRIGFGSFVEKTVMPYISTTPAKLRNPCTSEQNCTSPFSYKNVLSLTNKGEVFNELVGKQRISGNLDSPEGGFDAIMQVAVCGSLIGWRNVTRLLVFSTDAGFHFAGDGKLGGIVLPNDGQCHLENNMYTMSHYYDYPSIAHLVQKLSENNIQTIFAVTEEFQPVYKELKNLIPKSAVGTLSANSSNVIQLIIDAYNSLSSEVILENGKLSEGVTISYKSYCKNGVNGTGENGRKCSNISIGDEVQFEISITSNKCPKKDSDSFKIRPLGFTEEVEVILQYICECECQSEGIPESPKCHEGNGTFECGACRCNEGRVGRHCECSTDEVNSEDMDAYCRKENSSEICSNNGECVCGQCVCRKRDNTNEIYSGKFCECDNFNCDRSNGLICGGNGVCKCRVCECNPNYTGSACDCSLDTSTCEASNGQICNGRGICECGVCKCTDPKFQGQTCEMCQTCLGVCAEHKECVQCRAFNKGEKKDTCTQECSYFNITKVESRDKLPQPVQPDPVSHCKEKDVDDCWFYFTYSVNGNNEVMVHVVENPECPTGPDIIPIVAGVVAGIVLIGLALLLIWKLLMIIHDRREFAKFEKEKMNAKWDTVSYKTSKKQSGL

SEQ ID NO: 11 - protein, 825 aa, Homo sapiens
MNLQPIFWIGLISSVCCVFAQTDENRCLKANAKSCGECIQAGPNCGWCTNSTFLQEGMPTSARCDDLEALKKKGCPPDDIENPRGSKDIKKNKNVTNRSKGTAEKLKPEDITQIQPQQLVLRLRSGEPQTFTLKFKRAEDYPIDLYYLMDLSYSMKDDLENVKSLGTDLMNEMRRITSDFRIGFGSFVEKTVMPYISTTPAKLRNPCTSEQNCTSPFSYKNVLSLTNKGEVFNELVGKQRISGNLDSPEGGFDAIMQVAVCGSLIGWRNVTRLLVFSTDAGFHFAGDGKLGGIVLPNDGQCHLENNMYTMSHYYDYPSIAHLVQKLSENNIQTIFAVTEEFQPVYKELKNLIPKSAVGTLSANSSNVIQLIIDAYNSLSSEVILENGKLSEGVTISYKSYCKNGVNGTGENGRKCSNISIGDEVQFEISITSNKCPKKDSDSFKIRPLGFTEEVEVILQYICECECQSEGIPESPKCHEGNGTFECGACRCNEGRVGRHCECSTDEVNSEDMDAYCRKENSSEICSNNGECVCGQCVCRKRDNTNEIYSGKFCECDNFNCDRSNGLICGGNGVCKCRVCECNPNYTGSACDCSLDTSTCEASNGQICNGRGICECGVCKCTDPKFQGQTCEMCQTCLGVCAEHKECVQCRAFNKGEKKDTCTQECSYFNITKVESRDKLPQPVQPDPVSHCKEKDVDDCWFYFTYSVNGNNEVMVHVVENPECPTGPDIIPIVAGVVAGIVLIGLALLLIWKLLMIIHDRREFAKFEKEKMNAKWDTSLSVAQPGVQWCDISSLQPLTSRFQQFSCLSLPSTWDYRVKILFIRVP

SEQ ID NO: 12 - protein, 819 aa, Homo sapiens
MNLQPIFWIGLISSVCCVFAQTDENRCLKANAKSCGECIQAGPNCGWCTNSTFLQEGMPTSARCDDLEALKKKGCPPDDIENPRGSKDIKKNKNVTNRSKGTAEKLKPEDITQIQPQQLVLRLRSGEPQTFTLKFKRAEDYPIDLYYLMDLSYSMKDDLENVKSLGTDLMNEMRRITSDFRIGFGSFVEKTVMPYISTTPAKLRNPCTSEQNCTSPFSYKNVLSLTNKGEVFNELVGKQRISGNLDSPEGGFDAIMQVAVCGSLIGWRNVTRLLVFSTDAGFHFAGDGKLGGIVLPNDGQCHLENNMYTMSHYYDYPSIAHLVQKLSENNIQTIFAVTEEFQPVYKELKNLIPKSAVGTLSANSSNVIQLIIDAYNSLSSEVILENGKLSEGVTISYKSYCKNGVNGTGENGRKCSNISIGDEVQFEISITSNKCPKKDSDSFKIRPLGFTEEVEVILQYICECECQSEGIPESPKCHEGNGTFECGACRCNEGRVGRHCECSTDEVNSEDMDAYCRKENSSEICSNNGECVCGQCVCRKRDNTNEIYSGKFCECDNFNCDRSNGLICGGNGVCKCRVCECNPNYTGSACDCSLDTSTCEASNGQICNGRGICECGVCKCTDPKFQGQTCEMCQTCLGVCAEHKECVQCRAFNKGEKKDTCTQECSYFNITKVESRDKLPQPVQPDPVSHCKEKDVDDCWFYFTYSVNGNNEVMVHVVENPECPTGPDIIPIVAGVVAGIVLIGLALLLIWKLLMIIHDRREFAKFEKEKMNAKWDTPGVQWCDISSLQPLTSRFQQFSCLSLPSTWDYRVKILFIRVP

SEQ ID NO: 13 - protein, 801 aa, Homo sapiens
MNLQPIFWIGLISSVCCVFAQTDENRCLKANAKSCGECIQAGPNCGWCTNSTFLQEGMPTSARCDDLEALKKKGCPPDDIENPRGSKDIKKNKNVTNRSKGTAEKLKPEDITQIQPQQLVLRLRSGEPQTFTLKFKRAEDYPIDLYYLMDLSYSMKDDLENVKSLGTDLMNEMRRITSDFRIGFGSFVEKTVMPYISTTPAKLRNPCTSEQNCTSPFSYKNVLSLTNKGEVFNELVGKQRISGNLDSPEGGFDAIMQVAVCGSLIGWRNVTRLLVFSTDAGFHFAGDGKLGGIVLPNDGQCHLENNMYTMSHYYDYPSIAHLVQKLSENNIQTIFAVTEEFQPVYKELKNLIPKSAVGTLSANSSNVIQLIIDAYNSLSSEVILENGKLSEGVTISYKSYCKNGVNGTGENGRKCSNISIGDEVQFEISITSNKCPKKDSDSFKIRPLGFTEEVEVILQYICECECQSEGIPESPKCHEGNGTFECGACRCNEGRVGRHCECSTDEVNSEDMDAYCRKENSSEICSNNGECVCGQCVCRKRDNTNEIYSGKFCECDNFNCDRSNGLICGGNGVCKCRVCECNPNYTGSACDCSLDTSTCEASNGQICNGRGICECGVCKCTDPKFQGQTCEMCQTCLGVCAEHKECVQCRAFNKGEKKDTCTQECSYFNITKVESRDKLPQPVQPDPVSHCKEKDVDDCWFYFTYSVNGNNEVMVHVVENPECPTGPDIIPIVAGVVAGIVLIGLALLLIWKLLMIIHDRREFAKFEKEKMNAKWDTQENPIYKSPINNFKNPNYGRKAGL

SEQ ID NO: 14 - protein, 1066 aa, Homo sapiens
MGPGPSRAPRAPRLMLCALALMVAAGGCVVSAFNLDTRFLVVKEAGNPGSLFGYSVALHRQTERQQRYLLLAGAPRELAVPDGYTNRTGAVYLCPLTAHKDDCERMNITVKNDPGHHIIEDMWLGVTVASQGPAGRVLVCAHRYTQVLWSGSEDQRRMVGKCYVRGNDLELDSSDDWQTYHNEMCNSNTDYLETGMCQLGTSGGFTQNTVYFGAPGAYNWKGNSYMIQRKEWDLSEYSYKDPEDQGNLYIGYTMQVGSFILHPKNITIVTGAPRHRHMGAVFLLSQEAGGDLRRRQVLEGSQVGAYFGSAIALADLNNDGWQDLLVGAPYYFERKEEVGGAIYVFMNQAGTSFPAHPSLLLHGPSGSAFGLSVASIGDINQDGFQDIAVGAPFEGLGKVYIYHSSSKGLLRQPQQVIHGEKLGLPGLATFGYSLSGQMDVDENFYPDLLVGSLSDHIVLLRARPVINIVHKTLVPRPAVLDPALCTATSCVQVELCFAYNQSAGNPNYRRNITLAYTLEADRDRRPPRLRFAGSESAVFHGFFSMPEMRCQKLELLLMDNLRDKLRPIIISMNYSLPLRMPDRPRLGLRSLDAYPILNQAQALENHTEVQFQKECGPDNKCESNLQMRAAFVSEQQQKLSRLQYSRDVRKLLLSINVTNTRTSERSGEDAHEALLTLVVPPALLLSSVRPPGACQANETIFCELGNPFKRNQRMELLIAFEVIGVTLHTRDLQVQLQLSTSSHQDNLWPMILTLLVDYTLQTSLSMVNHRLQSFFGGTVMGESGMKTVEDVGSPLKYEFQVGPMGEGLVGLGTLVLGLEWPYEVSNGKWLLYPTEITVHGNGSWPCRPPGDLINPLNLTLSDPGDRPSSPQRRRRQLDPGGGQGPPPVTLAAAKKAKSETVLTCATGRAHCVWLECPIPDAPVVTNVTVKARVWNSTFIEDYRDFDRVRVNGWATLFLRTSIPTINMENKTTWFSVDIDSELVEELPAEIELWLVLVAVGAGLLLLGLIILLLWKCDFFKRTRYYQIMPKYHAVRIREEERYPPPGSTLPTKKHWVTSWQTRDQYY